(19) United States
(12) Patent Application Publication
(10) Pub. No.: US 2007/0010514 A1
(43) Pub. Date: Jan. 11, 2007

(54) Non-steroidal progesterone receptor modulators

(76) Inventors: Ulrich Bothe, Berlin (DE); Alexander Hillisch, Velbert (DE); Peter Droescher, Weimar (DE); Gunter Kaufmann, Jena (DE); Lothar Sobek, Jena (DE); Ulrike Fuhrmann, Berlin (DE); Norbert Schmees, Berlin (DE); Wolfgang Schwede, Glienicke (DE); Carsten Moeller, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/473,325

(22) Filed: Jun. 23, 2006

Related U.S. Application Data
(60) Provisional application No. 60/693,403, filed on Jun. 24, 2005.

(30) Foreign Application Priority Data
Jun. 24, 2005 (DE) 102005030292.0-44

(51) Int. Cl. A61K 31/538; C07D 265/02

(52) U.S. Cl. 514/230.5; 544/63

(57) ABSTRACT

The present invention relates to non-steroidal progesterone receptor modulators of the general formula I, 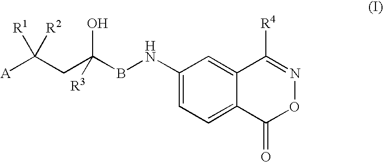 a process for their preparation, the use of the progesterone receptor modulators for producing medicaments, and pharmaceutical compositions comprising these compounds. The compounds according to the invention are suitable for the therapy and prophylaxis of gynaecological disorders such as endometriosis, leiomyomas of the uterus, dysfunctional bleeding and dysmenorrhoea, and for the therapy and prophylaxis of hormone-dependent tumours and for use for female fertility control and for hormone replacement therapy.

This application claims the benefit of the filing date of U.S. Provisional Application Ser. No. 60/693,403 filed Jun. 24, 2005.

The present invention relates to non-steroidal progesterone receptor modulators, to a process for their preparation, to the use of the progesterone receptor modulators for producing medicaments, and to pharmaceutical compositions which comprise these compounds.

The steroid hormone progesterone controls in a decisive manner the reproductive process in the female body. Progesterone is secreted in large quantities during the cycle and pregnancy respectively by the ovary and the placenta. Progesterone in cooperation with oestrogens brings about cyclic changes in the uterine mucosa (endometriur) during the menstrual cycle. Elevated progesterone levels after ovulation influence the uterine mucosa to convert it into a state permitting nidation of an embryo (blastocyst). During pregnancy, progesterone controls the relaxation of the myometrium and maintains the function of the decidual tissue.

It is further known that progesterone inhibits endometrial proliferation by suppressing oestrogen-mediated mitosis in uterine tissue (K. Chwalisz, R. M. Brenner, U. Fuhrmann, H. Hess-Stumpp, W. Elger, Steroids 65, 2000, 741-751).

Progesterone and progesterone receptors are also known to play a significant part in pathophysiological processes. Progesterone receptors have been detected in the foci of endometriosis, but also in tumours of the uterus, of the breast and of the CNS. It is further known that uterine leiomyomas grow progesterone-dependently.

The effects of progesterone in the tissues of the genital organs and in other tissues occur through interactions with progesterone receptors which are responsible for the cellular effects.

Progesterone receptor modulators are either pure agonists or inhibit the effect of progesterone partly or completely. Accordingly, substances are defined as pure agonists, partial agonists (SPRMs) and pure antagonists.

In accordance with ability of progesterone receptor modulators to influence the effect of the progesterone receptor, these compounds have a considerable potential as therapeutic agents for gynaecological and oncological indications and for obstetrics and fertility control.

Pure progesterone receptor antagonists completely inhibit the effect of progesterone on the progesterone receptor. They have anti-ovulatory properties and the ability to inhibit oestrogen effects in the endometrium, as far as complete atrophy. They are therefore particularly suitable for intervening in the female reproductive process, e.g. post-ovulation, in order to prevent nidation, during pregnancy in order to increase the reactivity of the uterus to prostaglandins or oxytocin, or in order to achieve opening and softening (“ripening”) of the cervix, and to induce a great, readiness of myometrium to contract.

A beneficial effect on the pathological event is expected in foci of endometriosis and in tumour tissues which are equipped with progesterone receptors after administration of pure progesterone receptor antagonists. There might be particular advantages for influencing pathological states such as endometriosis or uterine leiomyomas if ovulation inhibition can additionally be achieved by the progesterone receptor antagonists. Ovulation inhibition also dispenses with some of the ovarian hormone production and thus the stimulating effect, deriving from this proportion, on the pathologically altered tissue.

The first progesterone receptor antagonist described, RU 486 (also mifepristone), was followed by a large number of analogues with progesterone receptor-antagonistic activity of varying strength. Whereas RU 486 shows an antiglucocorticoid effect in addition to the progesterone receptor-antagonistic effect, compounds synthesized later are notable in particular for a more selective effect as progesterone receptor antagonists.

Besides steroidal compounds such as onapristone or lilopristone, which are notable by comparison with RU 486 for a better dissociation of the progesterone receptor-antagonistic effect and the antiglucocorticoid effect, also known from the literature are various non-steroidal structures whose antagonistic effect on the progesterone receptor is being investigated [see, for example, S. A. Leonhardt and D. P. Edwards, Exp. Biol. Med. 227: 969-980 (2002) and R. Winneker, A. Fensome, J. E. Wrobel, Z. Zhang, P. Zhang, Seminars in Reproductive Medicine, Volume 23: 46-57 (2005)]. However, compounds disclosed to date have only moderate antagonistic activity compared with the known steroidal structures. The most effective non-steroidal compounds are reported to have in vitro activities which are 10% of the activity of RU 486.

The antiglucocorticoid activity is disadvantageous for therapeutic use, where the inhibition of progesterone receptors is at the forefront of the therapy. An antiglucocorticoid activity causes unwanted side effects at the dosages necessary for therapy. This may prevent administration of a therapeutically worthwhile dose or lead to discontinuation of the treatment.

Partial or complete reduction of the antiglucocorticoid properties is therefore an important precondition for therapy with progesterone receptor antagonists, especially for those indications requiring treatment lasting weeks or months.

In contrast to the pure antagonists, partial progesterone receptor agonists (SPRMs) show a residual agonistic property which may vary in strength. This leads to these substances showing potentially agonistic effects on the progesterone receptor in certain organ systems (D. DeManno, W. Elger, R. Garg, R. Lee, B. Schneider, H. Hess-Stumpp, G. Schuber, K. Chwalisz, Steroids 68, 2003, 1019-1032). Such an organ-specific and dissociated effect may be of therapeutic benefit for the described indications.

It is therefore an object of the present invention to provide further non-steroidal progesterone receptor modulators. These compounds are intended to have a reduced antiglucocorticoid effect and therefore be suitable for the therapy and prophylaxis of gynaecological disorders such as endometriosis, leiomyomas of the uterus, dysfunctional bleeding and dysmenorrhoea. The compounds according to the invention are additionally intended to be suitable for the therapy and prophylaxis of hormone-dependent tumours, for example of breast, endometrial, ovarian and prostate carcinomas. The compounds are intended furthermore to be suitable for use in female fertility control and for female hormone replacement therapy.

The object is achieved according to the present invention by the provision of non-steroidal compounds of the general formula I 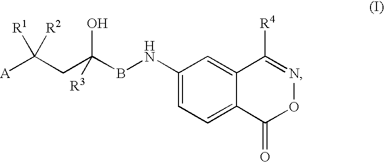
in which
R¹ and R² are independently of one another a hydrogen atom, a linear or nonlinear, branched or unbranched C₁-C₅-alkyl group, further forming together with the C atom of the chain a ring having a total of 3-7 members,
R³ is a radical C≡C—R^{a}, where
   R^{a} is a hydrogen or a C₁-C₈-alkyl, C₂-C₈-alkenyl, C₂-C₈-alkynyl, C₃-C₁₀-cycloalkyl, heterocycloalkyl optionally substituted one or more times, identically or differently, by K, or an aryl or heteroaryl optionally substituted one or more times, identically or differently by L,
      K is a cyano, halogen, hydroxy, nitro, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}—NR^{c}R^{d} or C₃-C₁₀-cycloalkyl, heterocycloalkyl optionally substituted one or more times, identically or differently, by M, or aryl or heteroaryl optionally substituted one or more times by L,
      L is C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚPO₃(R^{b})₂,
         —(CH₂)ₚNR^{c}R^{d}, (CH₂)NR^{e}COR^{b}, —(CH₂)ₚNR^{e}CSR^{b}, (CH₂)ₚNR^{e}S(O)R^{b}, —(CH₂)ₚNR^{e}S(O)₂R^{b}; —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚSR^{b}, —(CH₂)PS(O)R^{b}, —(CH₂)PS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{b}, —(CH₂)ₚCONR^{c}R^{d}, —(CH₂)ₚCSNR^{c}R^{d}, —(CH₂)ₚOR^{b}, —(CH₂)ₚSR^{b}, —(CH₂)ₚCR^{b}(OH)—Re, —(CH₂)ₚ—C═NOR^{b}, —O—(CH₂), —O—, —O—(CH₂), —CH₂—, —O—CH═CH— or —(CH₂)ₙ₊₂—, where n is 1 or 2, and the terminal oxygen atoms and/or carbon atoms are linked to directly adjacent ring carbon atoms,
      M is C₁-C₆-alkyl or a group —COR^{b}, CO₂R^{b}, —O—R^{b}, or —NR^{c}R^{d}, where
         R^{b} is a hydrogen or a C₁-C₆-alkyl, C₂-C₈-alkenyl, C₂-C₈-alkynyl, C₃-C₁₀-cycloalkyl, C₆-C₁₂-aryl or C₁-C₃-perfluoroalkyl and
         R^{c} and R^{d} are independently of one another a hydrogen, C₁-C₆-alkyl, C₂-C₈-alkenyl, C₂-C₈-alkynyl, C₃-C₁₀-cycloalkyl, C₆-C₁₂-aryl, C(O)R^{b} or a hydroxy group, where if
         R^{c} is a hydroxy group, then R^{d} can only be a hydrogen, a C₁-C₆-alkyl, C₂-C₈-alkenyl, C₂-C₈-alkynyl, C₃-C₁₀-cycloalkyl or C₆-C₁₂-aryl and vice versa, and
         R^{e} is a hydrogen, C₁-C₆-alkyl, C₂-C₈-alkenyl, C₂-C₈-alkynyl, C₃-C₁₀-cycloalkyl or C₆-C₁₂-aryl, and
         p can be a number from 0-6,
or
R³ is a radical C═C—R^{g}R^{h}, where
   R⁹ and R^{h} are independently of one another a hydrogen or a C₁-C₈-alkyl, C₂-C₈-alkenyl or C₂-C₈-alkynyl optionally substituted one or more times, identically or differently, by X, in which
      X is a cyano, halogen, hydroxy, nitro, —C(O)R^{b}, CO₂R^{b}, —O—R^{b}, —C(O)—NR^{c}R^{d}, —NR^{c}R^{d} with the meanings already mentioned-before for R^{b}, R^{c} and R^{d}, and
R⁴ is a hydrogen atom, a methyl or an ethyl group or a partly or completely fluorinated C₁-C₃-alkyl group,
A is a mono- or bicyclic carbocyclic or heterocyclic aromatic ring which may optionally be substituted one or more times by C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkyl, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl,
   —(CH₂)ₚPO₃(R^{b})₂, —(CH₂)ₚNR^{c}R^{d}, —(CH₂)ₚNR^{e}COR^{b}, (CH₂)ₚNR^{e}CSR^{b}; (CH₂)ₚNR^{e}S(O)R^{b}, —(CH₂)ₚNR^{e}S(O)₂R^{b}, —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚ S(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{b}, —(CH₂)ₚCONR^{c}R^{d}, —(CH₂)ₚCSNR^{c}R^{d}, —(CH₂)ₚOR^{b}, —(CH₂)ₚSR^{b}, —(CH₂)ₚCR^{b}(OH)—R^{d}, —(CH₂)ₚ—C═NOR^{b}, —O—(CH₂)ₙ—O—, —O—(CH₂), —CH₂—, —O—CH═CH— or —(CH₂)ₙ₊₂—, where n is 1 or 2, and the terminal oxygen atoms and/or carbon atoms are linked to directly adjacent ring carbon atoms, or
A is a radical —CO₂R^{b}, C(O)NR^{c}R^{d}, COR^{b},
or
A is an alkenyl group —CR⁵═CR⁶R⁷, where
   R⁵, R⁶ and R⁷ are identical or different and are independently of one another hydrogen atoms, halogen atoms, aryl radicals or an unsubstituted or partly or completely fluorinated C₁-C₅-alkyl group, or
A is an alkynyl group —C≡CR⁵, with the meaning stated above for R⁵, and
B is a carbonyl or a CH₂ group,
   and their pharmaceutically acceptable salts.

The compounds according to the invention of the general formula I may, owing to the presence of centres of asymmetry, exist as different stereoisomers. Both the racemates and the separate stereoisomers belong to the subject matter of the present invention.

The present invention further includes the novel compounds as active pharmaceutical ingredients, the preparation thereof, their therapeutic use and pharmaceutical dosage forms which comprise the novel substances.

The compounds according to the invention of the general formula (I) or their pharmaceutically acceptable salts can be used to produce a medicament, in particular for the treatment and prophylaxis of gynaecological disorders such as endometriosis, leiomyomas of the uterus, dysfunctional bleeding and dysmenorrhoea. The compounds according to the invention may further be used for the treatment and prophylaxis of hormone-dependent tumours such as, for example, for breast, prostate and endometrial carcinoma.

The compounds according to the invention of the general formula (I) or their pharmaceutically acceptable salts are suitable for use for female fertility control or for female hormone replacement therapy.

The present invention additionally relates to a process for preparing the compounds of the general formula (I). The substituent R³ is introduced by selective addition reaction of organometallic compounds such as lithium alkynyls or magnesium haloalkynyls onto a keto group. This leads either directly or after carrying out further modificiations to the compounds according to the invention of the general formula (I).

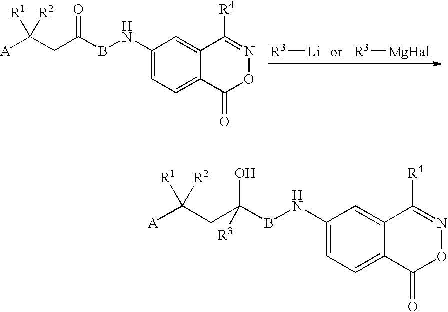

The compounds according to the invention are prepared by selective addition of organometallic compounds onto keto amides which have been described for example in the published specifications US 2002/0077356, U.S. Pat. No. 6,323,199B1, WO 200375915 and WO 9854159. The organometallic compounds may be for example lithium alkynyl or magnesium haloalkynyl compounds. These are generated for example by reacting the appropriate alkynes with butyllithium or Grignard compounds. The corresponding organometallic alkenyl compounds can also be prepared in analogy thereto. The reactivity of the keto groups is in this case distinctly higher by comparison with the amide carbonyl and with the benzoxazinone, so that a selective addition is achieved on suitable choice of the reaction conditions. Alternatively, the alkynyl or alkenyl radicals introduced as R³ can also be further modified later. Reactions suitable for these modifications are those known to the skilled person, such as oxidation, reduction, substitution, alkylation, palladium-catalysed reaction. Any protective groups present are eliminated at a suitable time.

The non-steroidal compounds according to the invention of the general formula I have strong antagonistic or strong partial agonistic effects on the progesterone receptor: They show a strong dissociation of effects in relation to their strength of binding to the progesterone receptor and to the glucocorticoid receptor. Whereas known progesterone receptor antagonists such as mifepristone (RU 486) show, besides the desired high binding affinity for the progesterone receptor, likewise a high affinity for the glucocorticoid receptor, the compounds according to the invention are notable for a very low glucocorticoid receptor binding with simultaneously a high progesterone receptor affinity.

The substituents, defined as groups, of the compounds according to the invention of the general formula I may in each case have the following meanings:

C₁-C₅-, C₁-C₆- and C₁-C₈-alkyl group means linear or nonlinear, branched or unbranched alkyl radicals. Examples thereof are a methyl, ethyl, n-propyl, isopropyl, n-, iso-, tert-butyl, an n-pentyl, 2,2-dimethylpropyl, 3-methylbutyl, hexyl, heptyl or octyl group.

Preferred in the meaning of R^{a} in this connection are the methyl, ethyl, n-propyl or n-butyl group and an n-pentyl group.

Preferred in the meaning of R¹ and R² are methyl or ethyl.

Alkenyl means linear or nonlinear, branched or unbranched alkenyl radicals. Examples of the meaning of a C₂-C₈-alkenyl group in the context of the invention are the following: vinyl, allyl, 3-buten-1-yl or 2,3-dimethyl-2-propenyl. If the aromatic system A is substituted by a C₂-C₈-alkenyl radical, it is preferably a vinyl group.

Alkynyl means linear or nonlinear, branched or unbranched alkynyl radicals. A C₂-C₈-alkynyl radical is intended to be for example an ethynyl, propynyl, butynyl, pentynyl, hexynyl and octynyl group, preferably an ethynyl or propynyl group.

Examples which may be mentioned of C₃-C₁₀-cycloalkyl are cyclopropyl, cyclobutyl, cyclopentyl and cyclohexyl. Cyclopropyl, cyclopentyl and cyclohexyl are preferred.

Heterocycloalkyl in the meaning of R^{a}, K and L means 3-8-membered heterocycloalkyl radicals. Examples of heterocycloalkyl are morpholinyl, tetrahydrofuranyl, pyranyl, piperazinyl, piperidinyl, pyrrolidinyl, oxiranyl, oxetanyl, aziridinyl, dioxolanyl and dioxanyl. In this connection, the position of the heteroatom in relation to the point of linkage can be any chemically possible position.

Possible examples of C₁-C₆-alkoxyl-C₁-C₆-alkoxy group are methoxymethoxy, ethoxymethoxy or 2-methoxyethoxy.

A radical OR^{b} in the context of the invention is a hydroxy, methoxy, ethoxy, n-propoxy, isopropoxy, n-, iso-, tert-butoxy or n-pentoxy, 2,2-dimethylpropoxy or 3-methylbutoxy group. Hydroxy, methoxy and ethoxy are preferred.

Suitable for a partly or completely fluorinated C₁-C₅-alkyl group are the perfluorinated alkyl groups above. Of these, preference is given in particular to the trifluoromethyl or pentafluoroethyl group and, partly fluorinated alkyl groups, for example the 5,5,5,4,4-pentafluoropentyl or 5,5,5,4,4,3,3-heptafluoropentyl group.

A halogen atom may be a fluorine, chlorine, bromine or iodine atom. Fluorine, chlorine or bromine is preferred here.

If R¹ and R² form together with the C atom of the chain a 3-7 membered ring, this is for example a cyclopropyl, -butyl, -pentyl or -hexyl ring. The cyclopropyl and the cyclopentyl ring are preferred.

The mono- or bicyclic carbocyclic aromatic ring A, which may be substituted more than once, is a carbocyclic or heterocyclic aryl radical.

In the former case it is for example a phenyl or naphthyl radical, preferably a phenyl radical.

It is possible to use as heterocyclic radical for example a monocyclic heterocyclic radical, for example the thienyl, furyl, pyranyl, pyrrolyl, imidazolyl, pyrazolyl, pyridyl, pyrazinyl, pyrimidinyl, pyridazinyl, thiazolyl, oxazolyl, furazanyl, pyrrolinyl, imidazolinyl, pyrazolinyl, thiazolinyl, triazolyl, tetrazolyl radical, in particular all the possible isomers in relation to the positions of the heteroatoms.

R³ means in the case of an aryl radical an optionally substituted phenyl, 1- or 2-naphthyl radical, with preference for the phenyl radical. Examples of a heteroaryl radical are the 2-, 3- or 4-pyridinyl, the 2- or 3-furyl, the 2- or 3-thienyl, the 2- or 3-pyrrolyl, the 2-, 4- or 5-imidazolyl, the pyrazinyl, the 2-, 4- or 5-pyrimidinyl or 3- or 4-pyridazinyl radical.

The number p for a (CH₂)ₚ radical may be a number from 0 to 6, preferably 0 to 2. “Radical” means according to the invention all functional groups stated in connection with (CH₂)ₚ.

In the case where the compounds of the general formula I (B=—CH₂—) are in the form of salts, this is possible for example in the form of the hydrochloride, sulphate, nitrate, tartrate, citrate, fumarate, succinate or benzoate.

If the compounds according to the invention are in the form of racemic mixtures, they can be fractionated by methods of racemate resolution familiar to the skilled person into the pure optically active forms. For example, the racemic mixtures can be separated into the pure isomers by chromatography on a support material which is itself optically active (CHIRALPAK AD®). It is also possible to esterify the free hydroxy group in a racemic compound of the general formula I with an optically active acid, and to separate the resulting diastereoisomeric esters by fractional crystallization or chromatography and to hydrolyse the separated esters in each case to the optically pure isomers. It is possible to use as optically active acid for example mandelic acid, camphorsulphonic acid or tartaric acid.

The compounds specified below, and the use thereof, are preferred according to the invention:

| | | |
|---|---|---|
| | Racemic or | |
| No. | enantiomer | R3 |

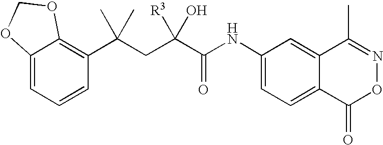

| | | |
|---|---|---|
| 1 2 3 | rac −+ | 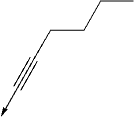 |
| 4 5 6 | rac −+ | 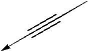 |
| 7 8 9 | rac −+ | 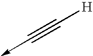 |
| 10 11 12 | rac −+ | 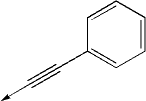 |
| 13 14 15 | rac −+ | 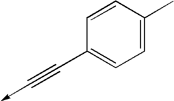 |
| 16 17 18 | rac −+ | 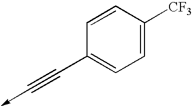 |
| 19 20 21 | rac −+ | 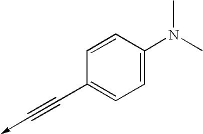 |
| 22 23 24 | rac −+ | 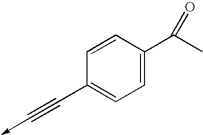 |
| 25 26 27 | rac −+ | 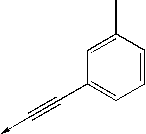 |
| 28 29 30 | rac −+ | 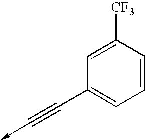 |
| 31 32 33 | rac −+ | 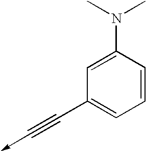 |
| 34 35 36 | rac −+ | 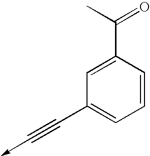 |
| 37 38 39 | rac −+ | 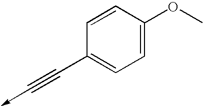 |
| 40 41 42 | rac −+ | 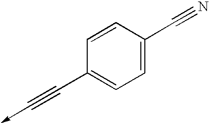 |
| 43 44 45 | rac −+ | 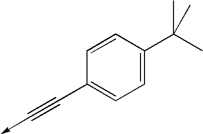 |
| 46 47 48 | rac −+ | 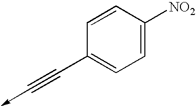 |
| 49 50 51 | rac −+ | 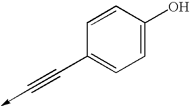 |

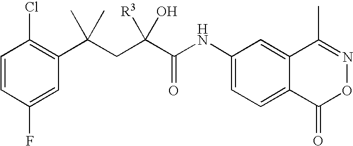

| | | |
|---|---|---|
| 52 53 54 | rac −+ | 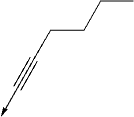 |
| 55 56 57 | rac −+ | 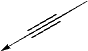 |
| 58 59 60 | rac −+ | 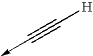 |
| 61 62 63 | rac −+ | 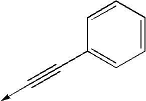 |
| 64 65 66 | rac −+ | 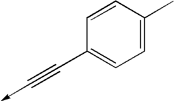 |
| 67 68 69 | rac −+ | 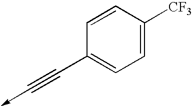 |
| 70 71 72 | rac −+ | 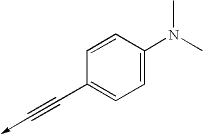 |
| 73 74 75 | rac −+ | 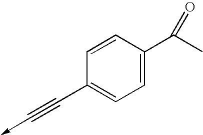 |
| 76 77 78 | rac −+ | 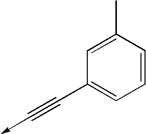 |
| 79 80 81 | rac −+ | 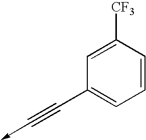 |
| 82 83 84 | rac −+ | 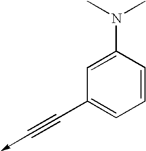 |
| 85 86 87 | rac −+ | 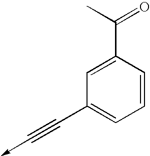 |
| 88 89 90 | rac −+ | 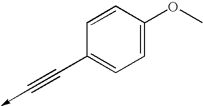 |
| 91 92 93 | rac −+ | 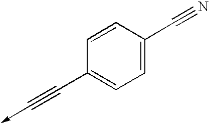 |
| 94 95 96 | rac −+ | 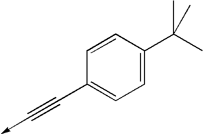 |
| 97 98 99 | rac −+ | 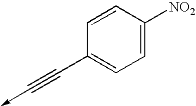 |
| 100 191 102 | rac −+ | 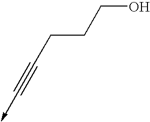 |
| 103 104 105 | rac −+ | 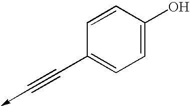 |

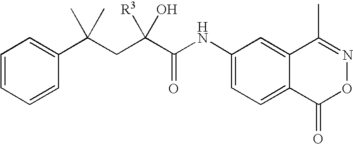

| | | |
|---|---|---|
| 106 107 108 | rac −+ | 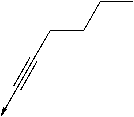 |
| 109 110 111 | rac −+ | 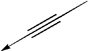 |
| 112 113 114 | rac −+ | 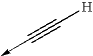 |
| 115 116 117 | rac −+ | 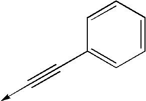 |
| 118 119 120 | rac −+ | 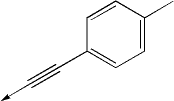 |
| 121 122 123 | rac −+ | 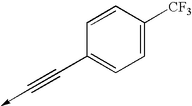 |
| 124 125 126 | rac −+ | 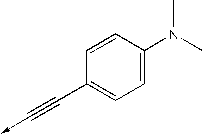 |
| 127 128 129 | rac −+ | 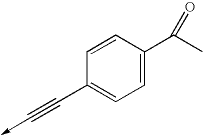 |
| 130 131 132 | rac −+ | 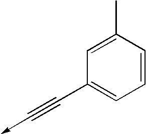 |
| 133 134 135 | rac −+ | 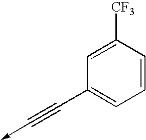 |
| 136 137 138 | rac −+ | 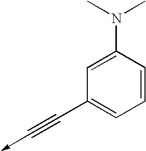 |
| 139 140 141 | rac −+ | 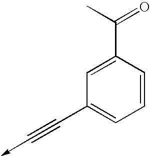 |
| 142 143 144 | rac −+ | 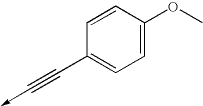 |
| 145 146 147 | rac −+ | 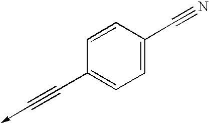 |
| 148 149 150 | rac −+ | 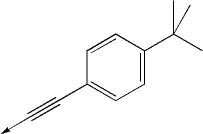 |
| 151 152 153 | rac −+ | 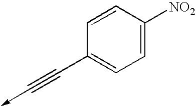 |
| 154 155 156 | rac −+ | 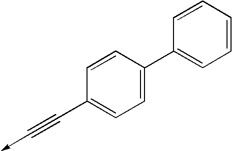 |
| 157 158 159 | rac −+ | 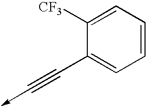 |
| 160 161 162 | rac −+ | 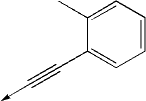 |
| 163 164 165 | rac −+ | 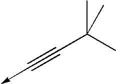 |
| 166 167 168 | rac −+ | 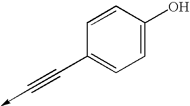 |

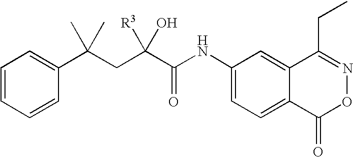

| | | |
|---|---|---|
| 169 170 171 | rac −+ | 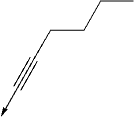 |
| 172 173 174 | rac −+ | 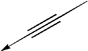 |
| 175 176 177 | rac −+ | 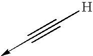 |
| 178 179 180 | rac −+ | 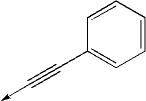 |
| 181 182 183 | rac −+ | 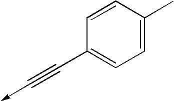 |
| 184 185 186 | rac −+ | 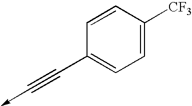 |
| 187 188 189 | rac −+ | 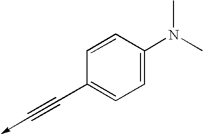 |
| 190 191 192 | rac −+ | 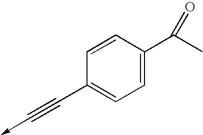 |
| 193 194 195 | rac −+ | 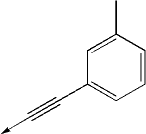 |
| 196 197 198 | rac −+ | 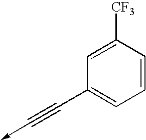 |
| 199 200 201 | rac −+ | 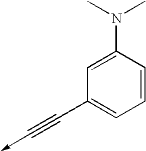 |
| 202 203 204 | rac −+ | 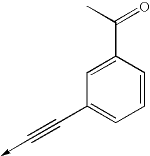 |
| 205 206 207 | rac −+ | 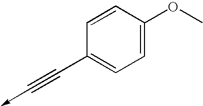 |
| 208 209 210 | rac −+ | 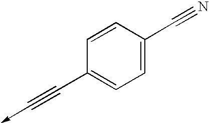 |
| 211 212 213 | rac −+ | 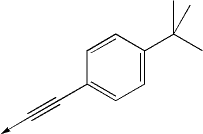 |
| 214 215 216 | rac −+ | 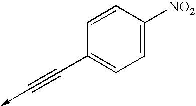 |
| 217 218 219 | rac −+ | 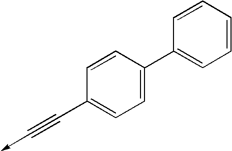 |
| 220 221 222 | rac −+ | 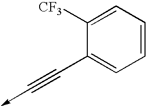 |
| 223 224 225 | rac −+ | 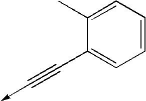 |
| 226 227 228 | rac −+ | 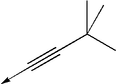 |
| 229 230 231 | rac −+ | 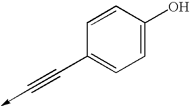 |

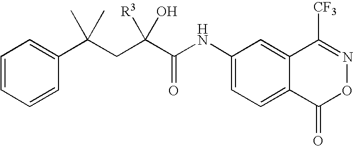

| | | |
|---|---|---|
| 232 233 234 | rac −+ | 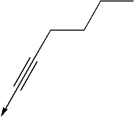 |
| 235 236 237 | rac −+ | 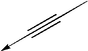 |
| 238 239 240 | rac −+ | 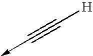 |
| 241 242 243 | rac −+ | 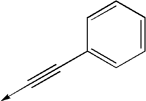 |
| 244 245 246 | rac −+ | 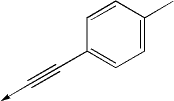 |
| 247 248 249 | rac −+ | 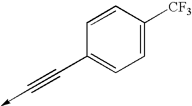 |
| 250 251 252 | rac −+ | 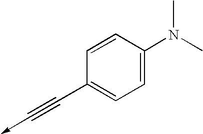 |
| 253 254 255 | rac −+ | 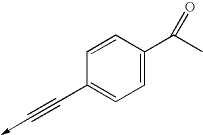 |
| 256 257 258 | rac −+ | 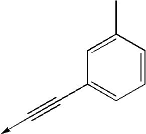 |
| 259 260 261 | rac −+ | 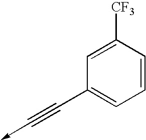 |
| 262 263 264 | rac −+ | 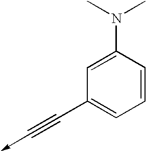 |
| 265 266 267 | rac −+ | 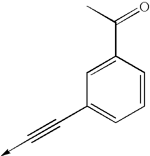 |
| 268 269 270 | rac −+ | 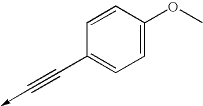 |
| 271 272 273 | rac −+ | 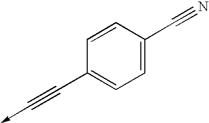 |
| 274 275 276 | rac −+ | 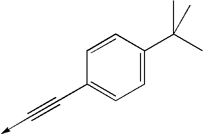 |
| 277 278 279 | rac −+ | 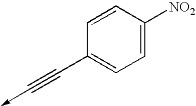 |
| 280 281 282 | rac −+ | 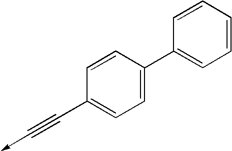 |
| 283 284 285 | rac −+ | 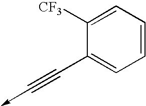 |
| 286 287 288 | rac −+ | 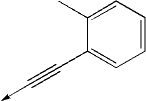 |
| 289 290 291 | rac −+ | 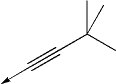 |
| 292 293 294 | rac −+ | 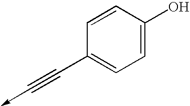 |

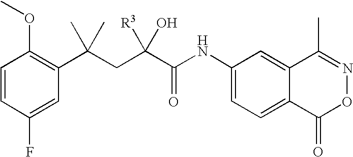

| | | |
|---|---|---|
| 295 296 297 | rac −+ | 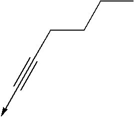 |
| 298 299 300 | rac −+ | 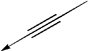 |
| 301 302 303 | rac −+ | 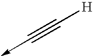 |
| 304 305 306 | rac −+ | 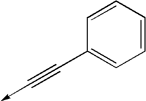 |
| 307 308 309 | rac −+ | 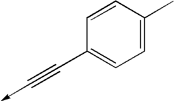 |
| 310 311 312 | rac −+ | 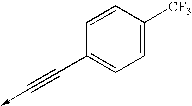 |
| 313 314 315 | rac −+ | 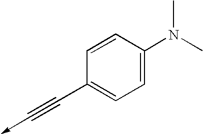 |
| 316 317 318 | rac −+ | 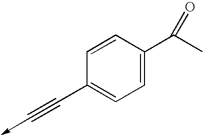 |
| 319 320 321 | rac −+ | 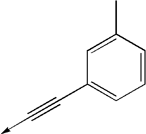 |
| 322 323 324 | rac −+ | 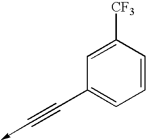 |
| 325 326 327 | rac −+ | 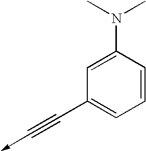 |
| 328 329 330 | rac −+ | 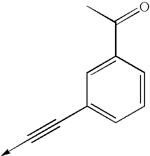 |
| 331 332 333 | rac −+ | 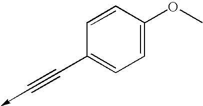 |
| 334 335 336 | rac −+ | 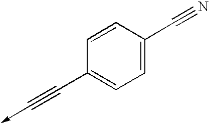 |
| 337 338 339 | rac −+ | 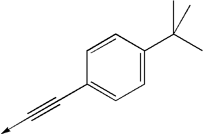 |
| 340 341 342 | rac −+ | 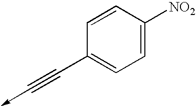 |
| 343 344 345 | rac −+ | 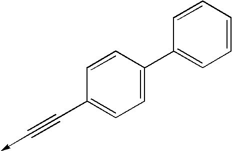 |
| 346 347 348 | rac −+ | 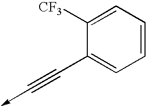 |
| 349 350 351 | rac −+ | 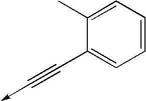 |
| 352 353 354 | rac −+ | 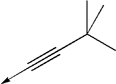 |
| 355 356 357 | rac −+ | 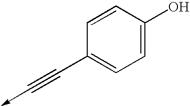 |

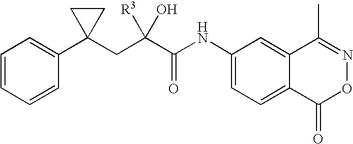

| | | |
|---|---|---|
| 358 359 360 | rac −+ | 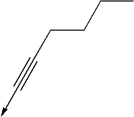 |
| 361 362 363 | rac −+ | 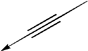 |
| 364 365 366 | rac −+ | 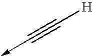 |
| 367 368 369 | rac −+ | 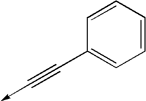 |
| 370 371 372 | rac −+ | 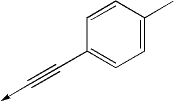 |
| 373 374 375 | rac −+ | 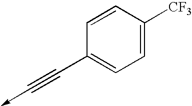 |
| 376 377 378 | rac −+ | 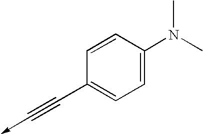 |
| 379 380 381 | rac −+ | 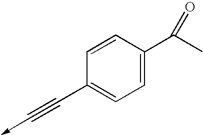 |
| 382 383 384 | rac −+ | 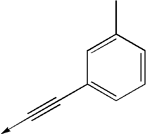 |
| 385 386 387 | rac −+ | 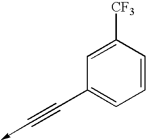 |
| 388 389 390 | rac −+ | 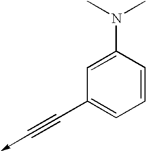 |
| 391 392 393 | rac −+ | 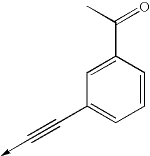 |
| 394 395 396 | rac −+ | 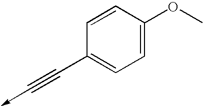 |
| 397 398 399 | rac −+ | 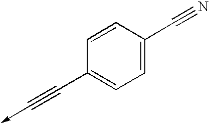 |
| 400 401 402 | rac −+ | 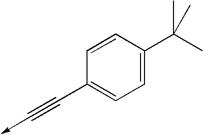 |
| 403 404 405 | rac −+ | 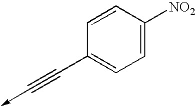 |
| 406 407 408 | rac −+ | 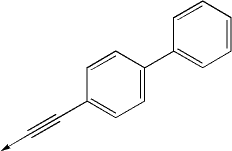 |
| 409 410 411 | rac −+ | 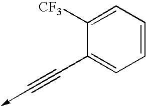 |
| 412 413 414 | rac −+ | 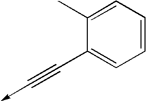 |
| 415 416 417 | rac −+ | 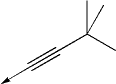 |
| 418 419 420 | rac −+ | 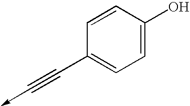 |

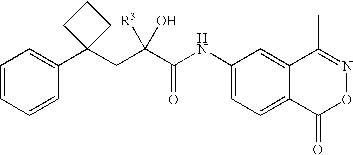

| | | |
|---|---|---|
| 421 422 423 | rac −+ | 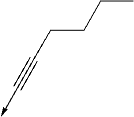 |
| 424 425 426 | rac −+ | 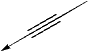 |
| 427 428 429 | rac −+ | 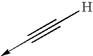 |
| 430 431 432 | rac −+ | 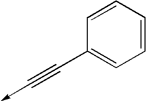 |
| 433 434 435 | rac −+ | 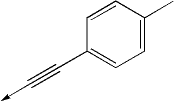 |
| 436 437 438 | rac −+ | 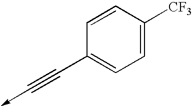 |
| 439 440 441 | rac −+ | 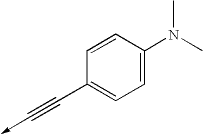 |
| 442 443 444 | rac −+ | 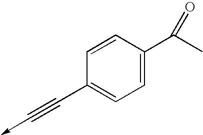 |
| 445 446 447 | rac −+ | 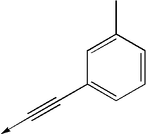 |
| 448 449 450 | rac −+ | 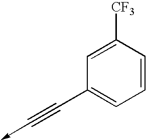 |
| 451 452 453 | rac −+ | 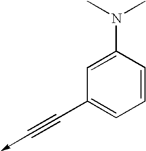 |
| 454 455 456 | rac −+ | 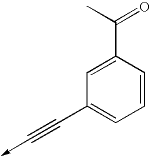 |
| 457 458 459 | rac −+ | 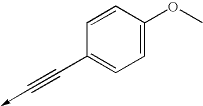 |
| 460 461 462 | rac −+ | 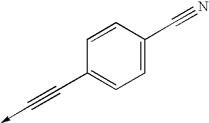 |
| 463 464 465 | rac −+ | 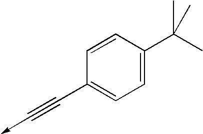 |
| 466 467 468 | rac −+ | 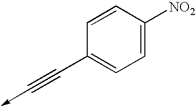 |
| 469 470 471 | rac −+ | 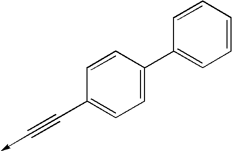 |
| 472 473 474 | rac −+ | 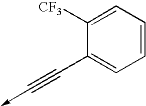 |
| 475 476 477 | rac −+ | 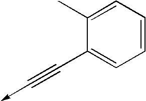 |
| 478 479 480 | rac −+ | 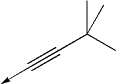 |
| 481 482 483 | rac −+ | 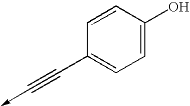 |

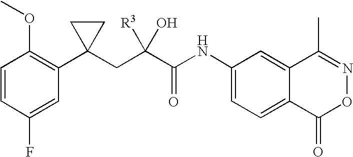

| | | |
|---|---|---|
| 484 485 486 | rac −+ | 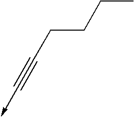 |
| 487 488 489 | rac −+ | 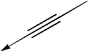 |
| 490 491 492 | rac −+ | 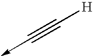 |
| 493 494 495 | rac −+ | 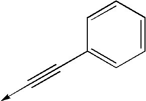 |
| 496 497 498 | rac −+ | 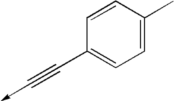 |
| 499 500 501 | rac −+ | 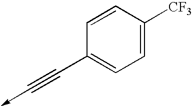 |
| 502 503 504 | rac −+ | 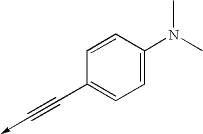 |
| 505 506 507 | rac −+ | 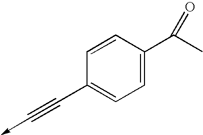 |
| 508 509 510 | rac −+ | 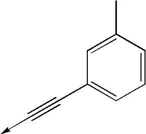 |
| 511 512 513 | rac −+ | 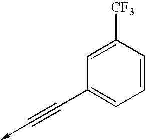 |
| 514 515 516 | rac −+ | 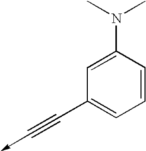 |
| 517 518 519 | rac −+ | 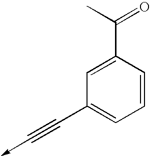 |
| 520 521 522 | rac −+ | 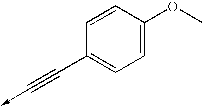 |
| 523 524 525 | rac −+ | 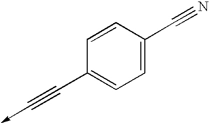 |
| 526 527 528 | rac −+ | 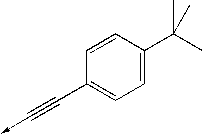 |
| 529 530 531 | rac −+ | 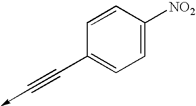 |
| 532 533 534 | rac −+ | 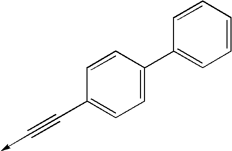 |
| 535 536 537 | rac −+ | 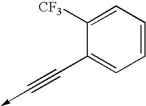 |
| 538 539 540 | rac −+ | 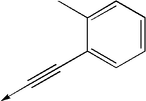 |
| 541 542 543 | rac −+ | 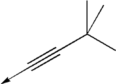 |
| 544 545 546 | rac −+ | 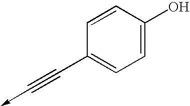 |

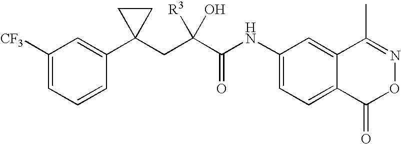

| | | |
|---|---|---|
| 547 548 549 | rac −+ | 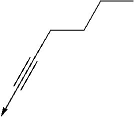 |
| 550 551 552 | rac −+ | 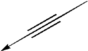 |
| 553 554 555 | rac −+ | 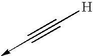 |
| 556 557 558 | rac −+ | 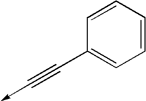 |
| 559 560 561 | rac −+ | 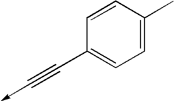 |
| 562 563 564 | rac −+ | 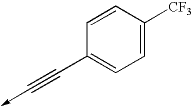 |
| 565 566 567 | rac −+ | 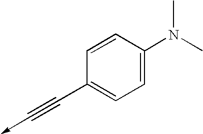 |
| 568 569 570 | rac −+ | 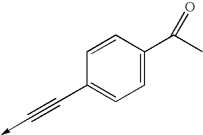 |
| 571 572 573 | rac −+ | 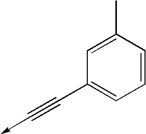 |
| 574 575 576 | rac −+ | 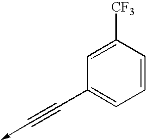 |
| 577 578 579 | rac −+ | 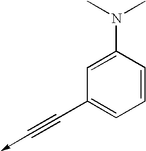 |
| 580 581 582 | rac −+ | 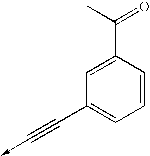 |
| 583 584 585 | rac −+ | 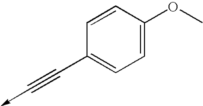 |
| 586 587 588 | rac −+ | 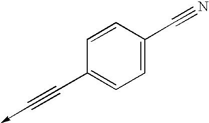 |
| 589 590 591 | rac −+ | 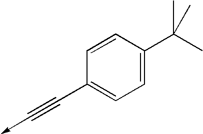 |
| 592 593 594 | rac −+ | 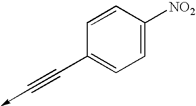 |
| 595 596 597 | rac −+ | 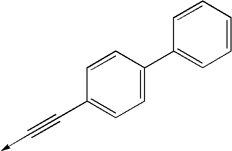 |
| 598 599 600 | rac −+ | 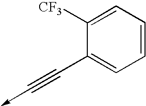 |
| 601 602 603 | rac −+ | 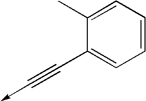 |
| 604 605 606 | rac −+ | 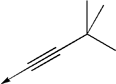 |
| 607 608 609 | rac −+ | 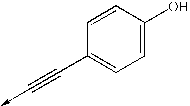 |

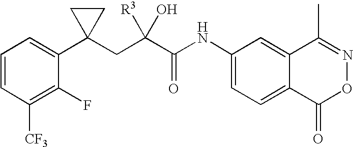

| | | |
|---|---|---|
| 610 611 612 | rac −+ | 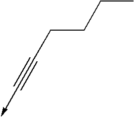 |
| 613 614 615 | rac −+ | 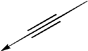 |
| 616 617 618 | rac −+ | 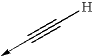 |
| 619 620 621 | rac −+ | 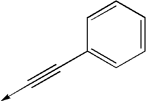 |
| 622 623 624 | rac −+ | 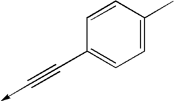 |
| 625 626 627 | rac −+ | 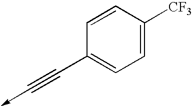 |
| 628 629 630 | rac −+ | 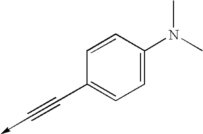 |
| 631 632 633 | rac −+ | 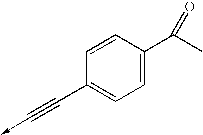 |
| 634 635 636 | rac −+ | 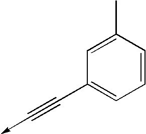 |
| 637 638 639 | rac −+ | 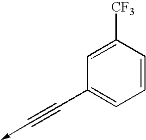 |
| 640 641 642 | rac −+ | 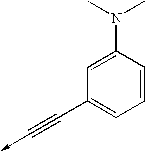 |
| 643 644 645 | rac −+ | 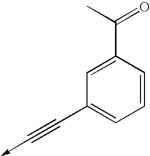 |
| 646 647 648 | rac −+ | 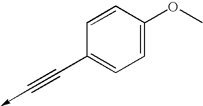 |
| 649 650 651 | rac −+ | 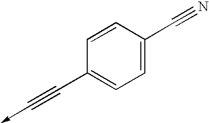 |
| 652 653 654 | rac −+ | 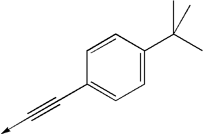 |
| 655 656 657 | rac −+ | 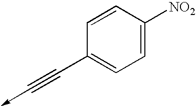 |
| 658 659 660 | rac −+ | 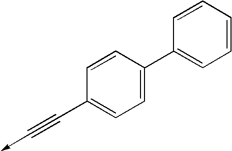 |
| 661 662 663 | rac −+ | 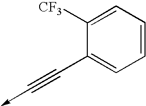 |
| 664 665 666 | rac −+ | 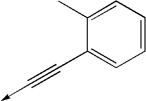 |
| 667 668 669 | rac −+ | 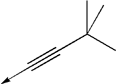 |
| 670 671 672 | rac −+ | 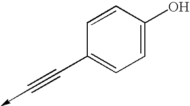 |

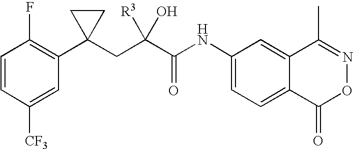

| | | |
|---|---|---|
| 673 674 675 | rac −+ | 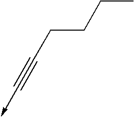 |
| 676 677 678 | rac −+ | 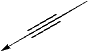 |
| 679 680 681 | rac −+ | 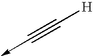 |
| 682 683 684 | rac −+ | 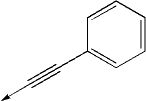 |
| 685 686 687 | rac −+ | 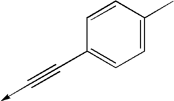 |
| 688 689 690 | rac −+ | 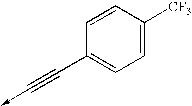 |
| 691 692 693 | rac −+ | 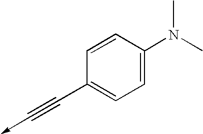 |
| 694 695 696 | rac −+ | 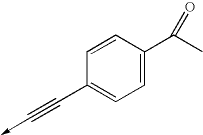 |
| 697 698 699 | rac −+ | 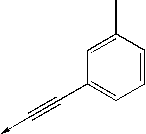 |
| 700 701 702 | rac −+ | 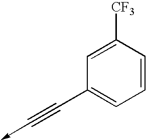 |
| 703 704 705 | rac −+ | 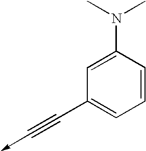 |
| 706 707 708 | rac −+ | 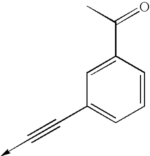 |
| 709 710 711 | rac −+ | 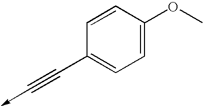 |
| 712 713 714 | rac −+ | 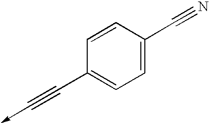 |
| 715 716 717 | rac −+ | 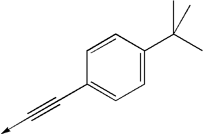 |
| 718 719 720 | rac −+ | 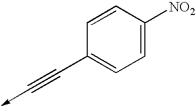 |
| 721 722 723 | rac −+ | 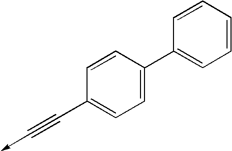 |
| 724 725 726 | rac −+ | 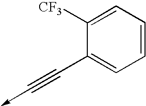 |
| 727 728 729 | rac −+ | 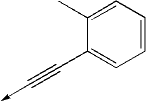 |
| 730 731 732 | rac −+ | 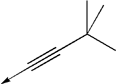 |
| 733 734 735 | rac −+ | 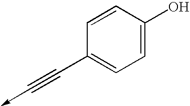 |

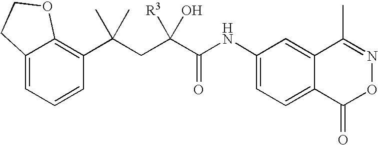

| | | |
|---|---|---|
| 736 737 738 | rac −+ | 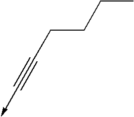 |
| 739 740 741 | rac −+ | 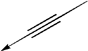 |
| 742 743 744 | rac −+ | 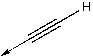 |
| 745 746 747 | rac −+ | 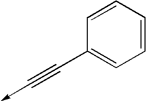 |
| 748 749 750 | rac −+ | 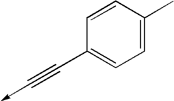 |
| 751 752 753 | rac −+ | 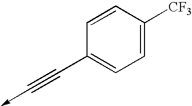 |
| 754 755 756 | rac −+ | 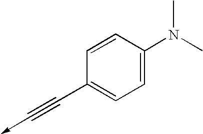 |
| 757 758 759 | rac −+ | 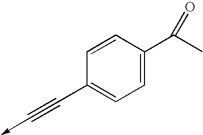 |
| 760 761 762 | rac −+ | 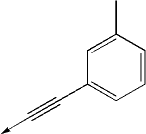 |
| 763 764 765 | rac −+ | 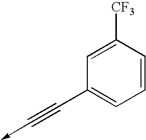 |
| 766 767 768 | rac −+ | 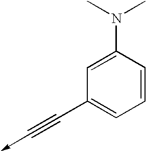 |
| 769 770 771 | rac −+ | 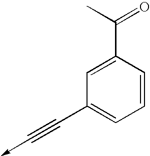 |
| 772 773 774 | rac −+ | 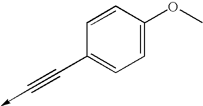 |
| 775 776 777 | rac −+ | 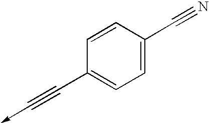 |
| 778 779 780 | rac −+ | 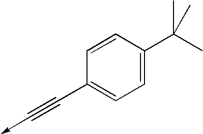 |
| 781 782 783 | rac −+ | 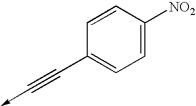 |
| 784 785 786 | rac −+ | 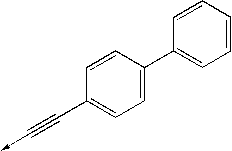 |
| 787 788 789 | rac −+ | 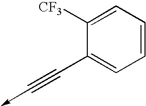 |
| 790 791 792 | rac −+ | 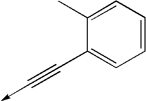 |
| 793 794 795 | rac −+ | 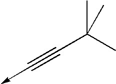 |
| 796 797 798 | rac −+ | 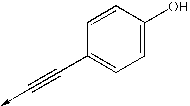 |

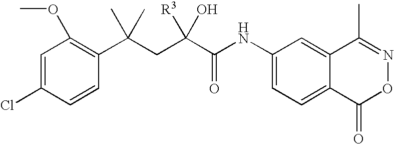

| | | |
|---|---|---|
| 799 800 801 | rac −+ | 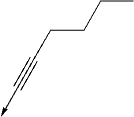 |
| 802 803 804 | rac −+ | 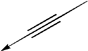 |
| 805 806 807 | rac −+ | 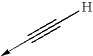 |
| 808 809 810 | rac −+ | 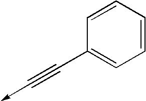 |
| 811 812 813 | rac −+ | 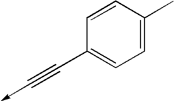 |
| 814 815 816 | rac −+ | 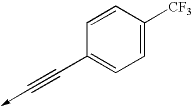 |
| 817 818 819 | rac −+ | 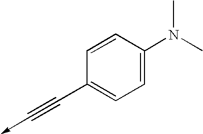 |
| 820 821 822 | rac −+ | 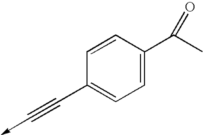 |
| 823 824 825 | rac −+ | 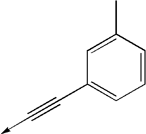 |
| 826 827 828 | rac −+ | 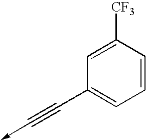 |
| 829 830 831 | rac −+ | 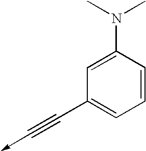 |
| 832 833 834 | rac −+ | 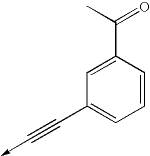 |
| 835 836 837 | rac −+ | 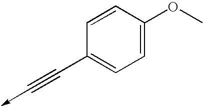 |
| 838 839 840 | rac −+ | 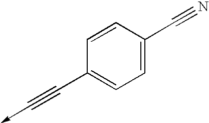 |
| 841 842 843 | rac −+ | 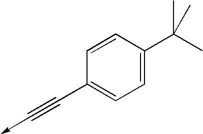 |
| 844 845 846 | rac −+ | 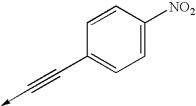 |
| 847 848 849 | rac −+ | 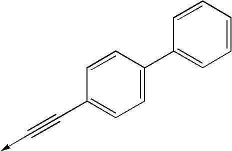 |
| 850 851 852 | rac −+ | 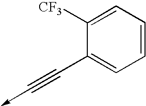 |
| 853 854 855 | rac −+ | 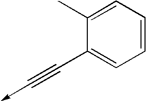 |
| 856 857 858 | rac −+ | 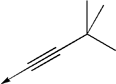 |
| 859 860 861 | rac −+ | 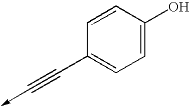 |

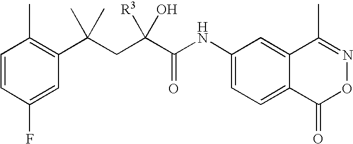

| | | |
|---|---|---|
| 862 863 864 | rac −+ | 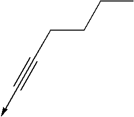 |
| 865 866 867 | rac −+ | 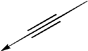 |
| 868 869 870 | rac −+ | 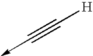 |
| 871 872 873 | rac −+ | 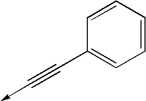 |
| 874 875 876 | rac −+ | 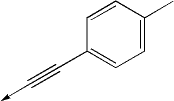 |
| 877 878 879 | rac −+ | 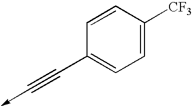 |
| 880 881 882 | rac −+ | 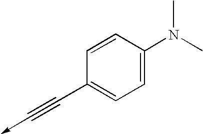 |
| 883 884 885 | rac −+ | 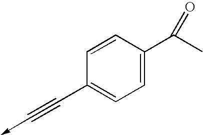 |
| 886 887 888 | rac −+ | 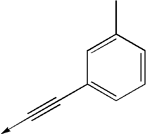 |
| 889 890 891 | rac −+ | 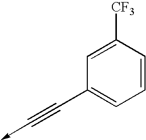 |
| 892 893 894 | rac −+ | 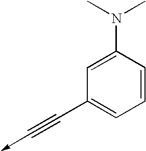 |
| 895 896 897 | rac −+ | 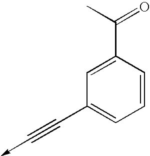 |
| 898 899 900 | rac −+ | 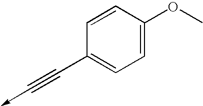 |
| 901 902 903 | rac −+ | 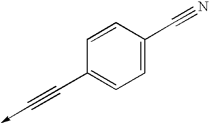 |
| 904 905 906 | rac −+ | 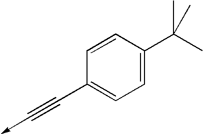 |
| 907 908 909 | rac −+ | 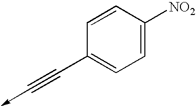 |
| 910 911 912 | rac −+ | 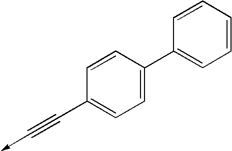 |
| 913 914 915 | rac −+ | 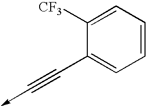 |
| 916 917 918 | rac −+ | 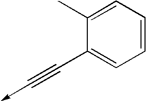 |
| 919 920 921 | rac −+ | 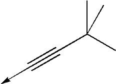 |
| 922 923 924 | rac −+ | 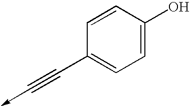 |

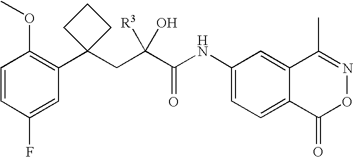

| | | |
|---|---|---|
| 925 926 927 | rac −+ | 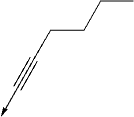 |
| 928 929 930 | rac −+ | 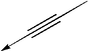 |
| 931 932 933 | rac −+ | 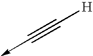 |
| 934 935 936 | rac −+ | 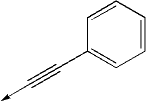 |
| 937 938 939 | rac −+ | 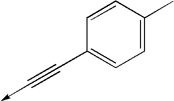 |
| 940 941 942 | rac −+ | 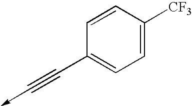 |
| 943 944 945 | rac −+ | 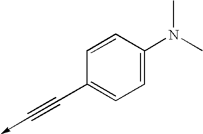 |
| 946 947 948 | rac −+ | 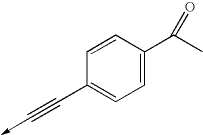 |
| 949 950 951 | rac −+ | 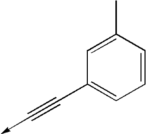 |
| 952 953 954 | rac −+ | 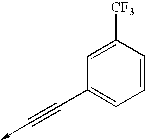 |
| 955 956 957 | rac −+ | 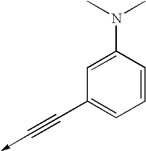 |
| 958 959 960 | rac −+ | 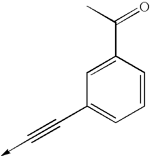 |
| 961 962 963 | rac −+ | 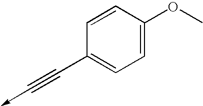 |
| 964 965 966 | rac −+ | 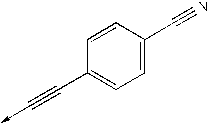 |
| 967 968 969 | rac −+ | 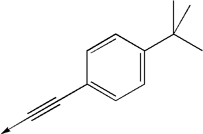 |
| 970 971 972 | rac −+ | 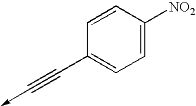 |
| 973 974 975 | rac −+ | 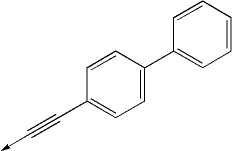 |
| 976 977 978 | rac −+ | 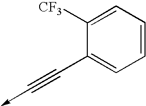 |
| 979 980 981 | rac −+ | 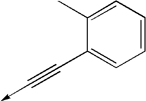 |
| 982 983 984 | rac −+ | 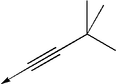 |
| 985 986 987 | rac −+ | 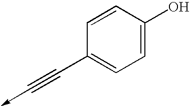 |

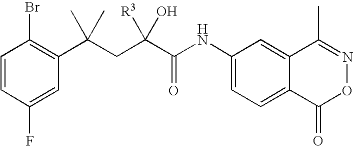

| | | |
|---|---|---|
| 988 989 990 | rac −+ | 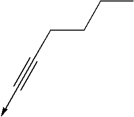 |
| 991 992 993 | rac −+ | 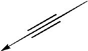 |
| 994 995 996 | rac −+ | 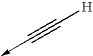 |
| 997 998 999 | rac −+ | 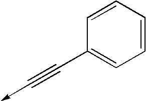 |
| 1000 1001 1002 | rac −+ | 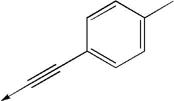 |
| 1003 1004 1005 | rac −+ | 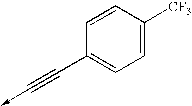 |
| 1006 1007 1008 | rac −+ | 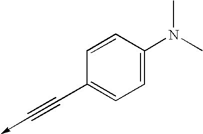 |
| 1009 1010 1011 | rac −+ | 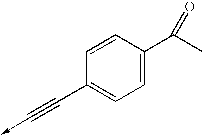 |
| 1012 1013 1014 | rac −+ | 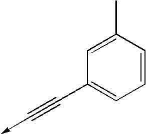 |
| 1015 1016 1017 | rac −+ | 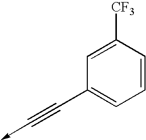 |
| 1018 1019 1020 | rac −+ | 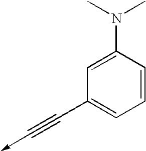 |
| 1021 1022 1023 | rac −+ | 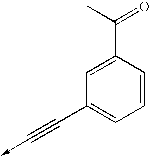 |
| 1024 1025 1026 | rac −+ | 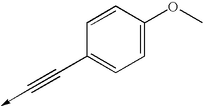 |
| 1027 1028 1029 | rac −+ | 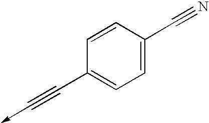 |
| 1030 1031 1032 | rac −+ | 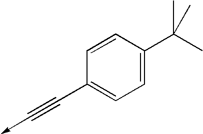 |
| 1033 1034 1035 | rac −+ | 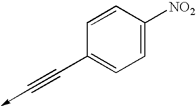 |
| 1036 1037 1038 | rac −+ | 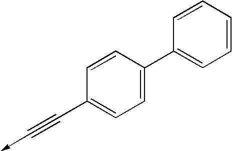 |
| 1039 1040 1041 | rac −+ | 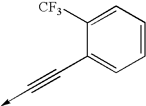 |
| 1042 1043 1044 | rac −+ | 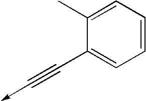 |
| 1045 1046 1047 | rac −+ | 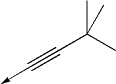 |
| 1048 1049 1050 | rac −+ | 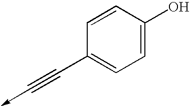 |

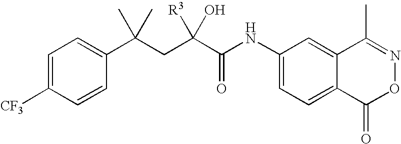

| | | |
|---|---|---|
| 1051 1052 1053 | rac −+ | 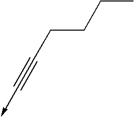 |
| 1054 1055 1056 | rac −+ | 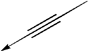 |
| 1057 1058 1059 | rac −+ | 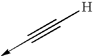 |
| 1060 1061 1062 | rac −+ | 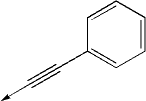 |
| 1063 1064 1065 | rac −+ | 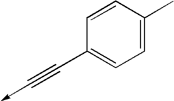 |
| 1066 1067 1068 | rac −+ | 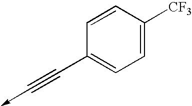 |
| 1069 1070 1071 | rac −+ | 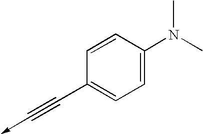 |
| 1072 1073 1074 | rac −+ | 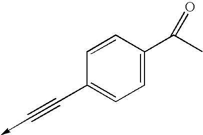 |
| 1075 1076 1077 | rac −+ | 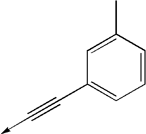 |
| 1078 1079 1080 | rac −+ | 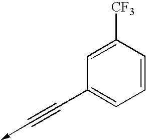 |
| 1081 1082 1083 | rac −+ | 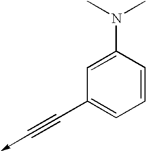 |
| 1084 1085 1086 | rac −+ | 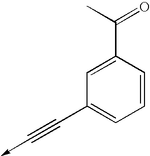 |
| 1087 1088 1089 | rac −+ | 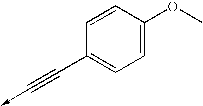 |
| 1090 1091 1092 | rac −+ | 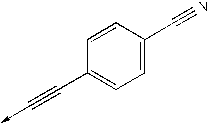 |
| 1093 1094 1095 | rac −+ | 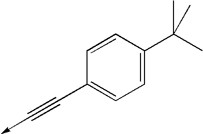 |
| 1096 1097 1098 | rac −+ | 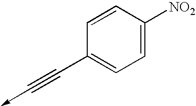 |
| 1099 1100 1101 | rac −+ | 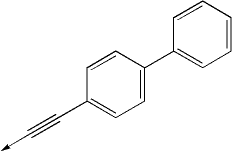 |
| 1102 1103 1104 | rac −+ | 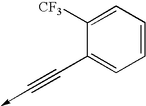 |
| 1105 1106 1107 | rac −+ | 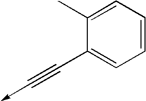 |
| 1108 1109 1110 | rac −+ | 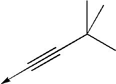 |
| 1111 1112 1113 | rac −+ | 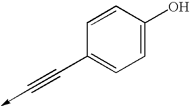 |

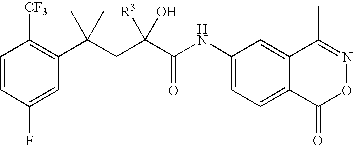

| | | |
|---|---|---|
| 1114 1115 1116 | rac −+ | 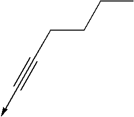 |
| 1117 1118 1119 | rac −+ | 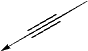 |
| 1120 1121 1122 | rac −+ | 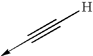 |
| 1123 1124 1125 | rac −+ | 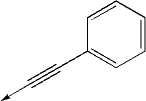 |
| 1126 1127 1128 | rac −+ | 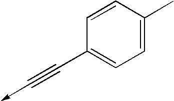 |
| 1129 1130 1131 | rac −+ | 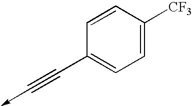 |
| 1132 1133 1134 | rac −+ | 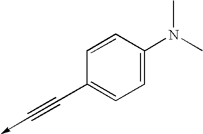 |
| 1135 1136 1137 | rac −+ | 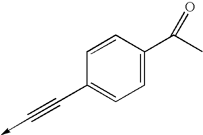 |
| 1138 1139 1140 | rac −+ | 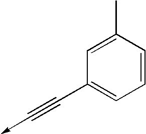 |
| 1141 1142 1143 | rac −+ | 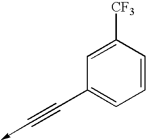 |
| 1144 1145 1146 | rac −+ | 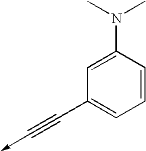 |
| 1147 1148 1149 | rac −+ | 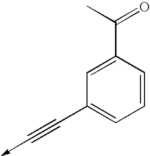 |
| 1150 1151 1152 | rac −+ | 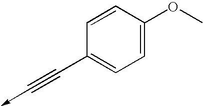 |
| 1153 1154 1155 | rac −+ | 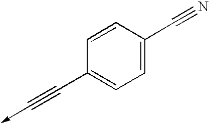 |
| 1156 1157 1158 | rac −+ | 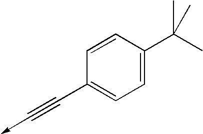 |
| 1159 1160 1161 | rac −+ | 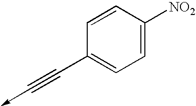 |
| 1162 1163 1164 | rac −+ | 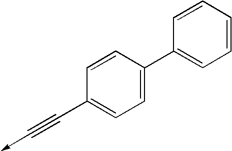 |
| 1165 1166 1167 | rac −+ | 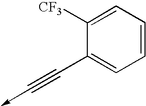 |
| 1168 1169 1170 | rac −+ | 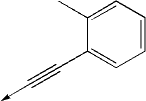 |
| 1171 1172 1173 | rac −+ | 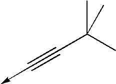 |
| 1174 1175 1176 | rac −+ | 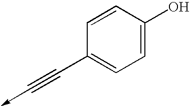 |

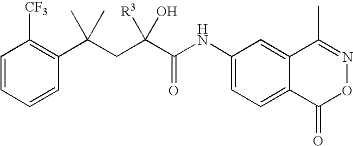

| | | |
|---|---|---|
| 1177 1178 1179 | rac −+ | 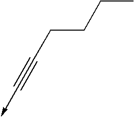 |
| 1180 1181 1182 | rac −+ | 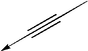 |
| 1183 1184 1185 | rac −+ | 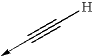 |
| 1186 1187 1188 | rac −+ | 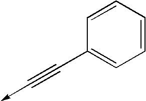 |
| 1189 1190 1191 | rac −+ | 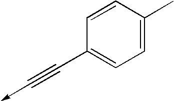 |
| 1192 1193 1194 | rac −+ | 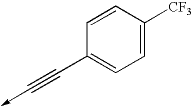 |
| 1195 1196 1197 | rac −+ | 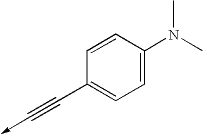 |
| 1198 1199 1200 | rac −+ | 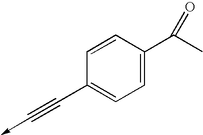 |
| 1202 1202 1203 | rac −+ | 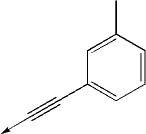 |
| 1204 1205 1206 | rac −+ | 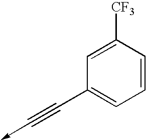 |
| 1207 1208 1209 | rac −+ | 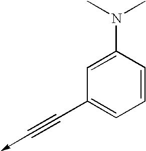 |
| 1210 1211 1212 | rac −+ | 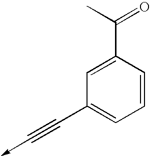 |
| 1213 1214 1215 | rac −+ | 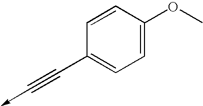 |
| 1216 1217 1218 | rac −+ | 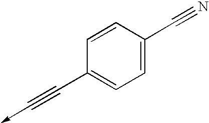 |
| 1219 1220 1221 | rac −+ | 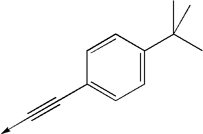 |
| 1222 1223 1224 | rac −+ | 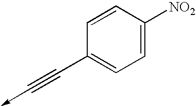 |
| 1225 1226 1227 | rac −+ | 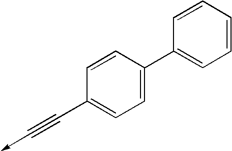 |
| 1228 1229 1230 | rac −+ | 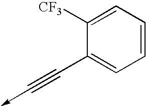 |
| 1231 1232 1233 | rac −+ | 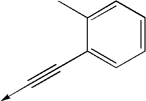 |
| 1234 1235 1236 | rac −+ | 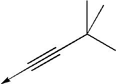 |
| 1237 1238 1239 | rac −+ | 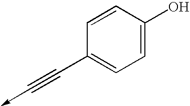 |

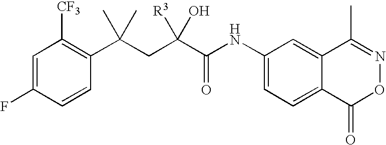

| | | |
|---|---|---|
| 1240 1241 1242 | rac −+ | 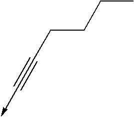 |
| 1243 1244 1245 | rac −+ | 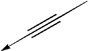 |
| 1246 1247 1248 | rac −+ | 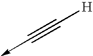 |
| 1249 1250 1251 | rac −+ | 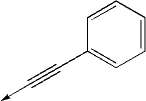 |
| 1252 1253 1254 | rac −+ | 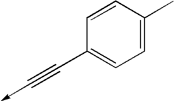 |
| 1255 1256 1257 | rac −+ | 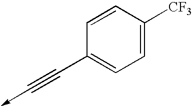 |
| 1258 1259 1260 | rac −+ | 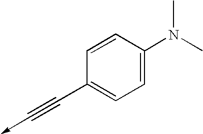 |
| 1261 1262 1263 | rac −+ | 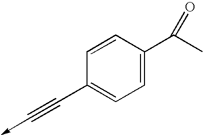 |
| 1264 1265 1266 | rac −+ | 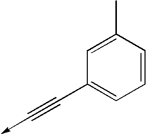 |
| 1267 1268 1269 | rac −+ | 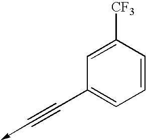 |
| 1270 1271 1272 | rac −+ | 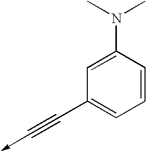 |
| 1273 1274 1275 | rac −+ | 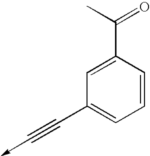 |
| 1276 1277 1278 | rac −+ | 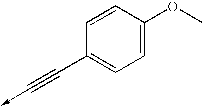 |
| 1279 1280 1281 | rac −+ | 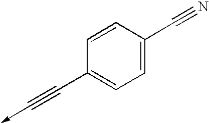 |
| 1282 1283 1284 | rac −+ | 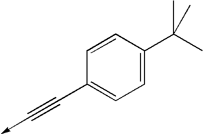 |
| 1285 1286 1287 | rac −+ | 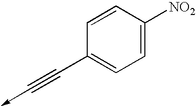 |
| 1288 1289 1290 | rac −+ | 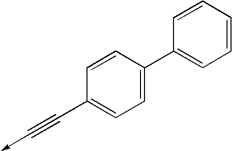 |
| 1291 1292 1293 | rac −+ | 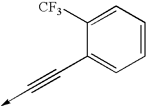 |
| 1294 1295 1296 | rac −+ | 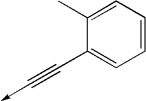 |
| 1297 1298 1299 | rac −+ | 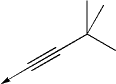 |
| 1300 1301 1302 | rac −+ | 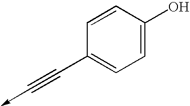 |

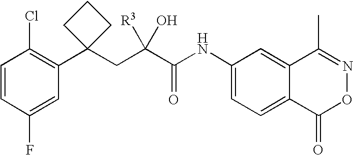

| | | |
|---|---|---|
| 1303 1304 1305 | rac −+ | 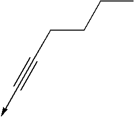 |
| 1306 1307 1308 | rac −+ | 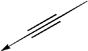 |
| 1309 1310 1311 | rac −+ | 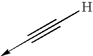 |
| 1312 1313 1314 | rac −+ | 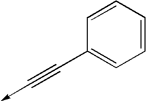 |
| 1315 1316 1317 | rac −+ | 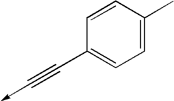 |
| 1318 1319 1320 | rac −+ | 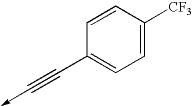 |
| 1321 1322 1323 | rac −+ | 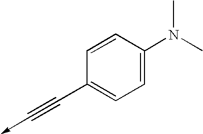 |
| 1324 1325 1326 | rac −+ | 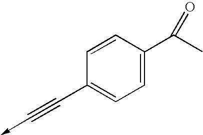 |
| 1327 1328 1329 | rac −+ | 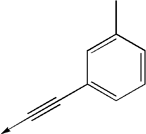 |
| 1330 1331 1332 | rac −+ | 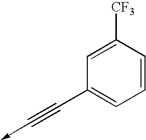 |
| 1333 1334 1335 | rac −+ | 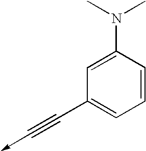 |
| 1336 1337 1338 | rac −+ | 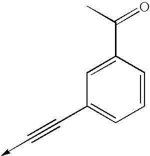 |
| 1339 1340 1341 | rac −+ | 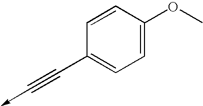 |
| 1342 1343 1344 | rac −+ | 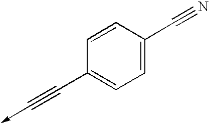 |
| 1345 1346 1347 | rac −+ | 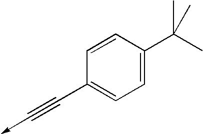 |
| 1348 1349 1350 | rac −+ | 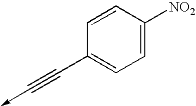 |
| 1351 1352 1353 | rac −+ | 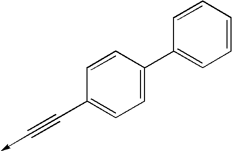 |
| 1354 1355 1356 | rac −+ | 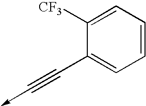 |
| 1357 1358 1359 | rac −+ | 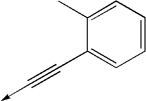 |
| 1360 1361 1362 | rac −+ | 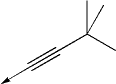 |
| 1363 1364 1365 | rac −+ | 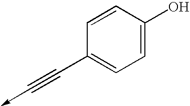 |

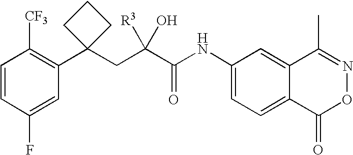

| | | |
|---|---|---|
| 1366 1367 1368 | rac −+ | 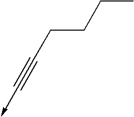 |
| 1369 1370 1371 | rac −+ | 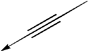 |
| 1372 1373 1374 | rac −+ | 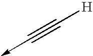 |
| 1375 1376 1377 | rac −+ | 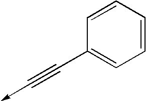 |
| 1378 1379 1380 | rac −+ | 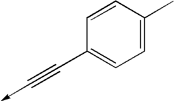 |
| 1381 1382 1383 | rac −+ | 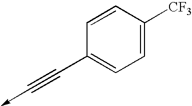 |
| 1384 1385 1386 | rac −+ | 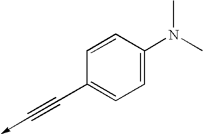 |
| 1387 1388 1389 | rac −+ | 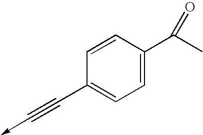 |
| 1390 1391 1392 | rac −+ | 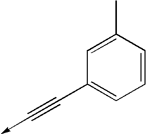 |
| 1393 1394 1395 | rac −+ | 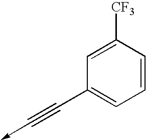 |
| 1396 1397 1398 | rac −+ | 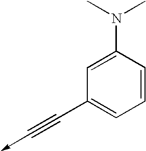 |
| 1399 1400 1401 | rac −+ | 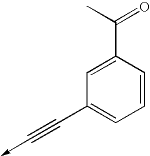 |
| 1402 1403 1404 | rac −+ | 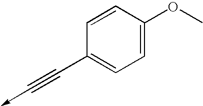 |
| 1405 1406 1407 | rac −+ | 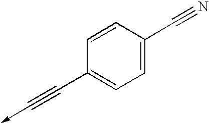 |
| 1408 1409 1410 | rac −+ | 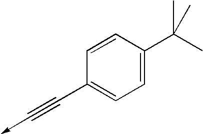 |
| 1411 1412 1413 | rac −+ | 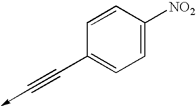 |
| 1414 1415 1416 | rac −+ | 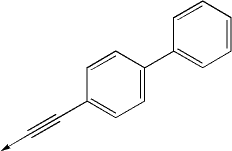 |
| 1417 1418 1419 | rac −+ | 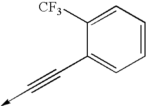 |
| 1420 1421 1422 | rac −+ | 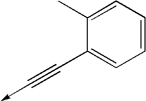 |
| 1423 1424 1425 | rac −+ | 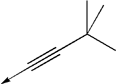 |
| 1426 1427 1428 | rac −+ | 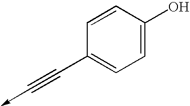 |

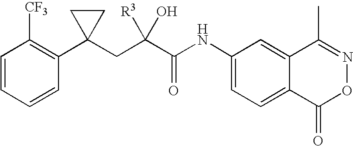

| | | |
|---|---|---|
| 1429 1430 1431 | rac −+ | 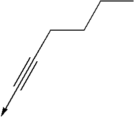 |
| 1432 1433 1434 | rac −+ | 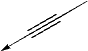 |
| 1435 1436 1437 | rac −+ | 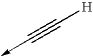 |
| 1438 1439 1440 | rac −+ | 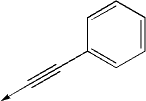 |
| 1441 1442 1443 | rac −+ | 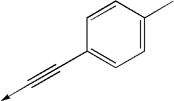 |
| 1444 1445 1446 | rac −+ | 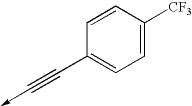 |
| 1447 1448 1449 | rac −+ | 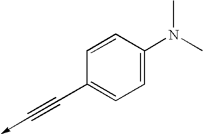 |
| 1450 1451 1452 | rac −+ | 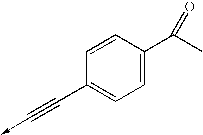 |
| 1453 1454 1455 | rac −+ | 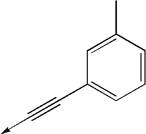 |
| 1456 1457 1458 | rac −+ | 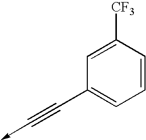 |
| 1459 1460 1461 | rac −+ | 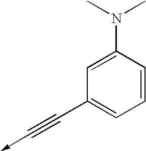 |
| 1462 1463 1464 | rac −+ | 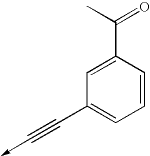 |
| 1465 1466 1467 | rac −+ | 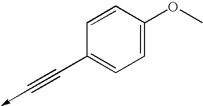 |
| 1468 1469 1470 | rac −+ | 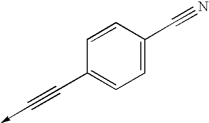 |
| 1471 1472 1473 | rac −+ | 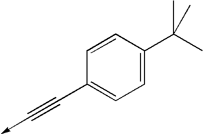 |
| 1474 1475 1476 | rac −+ | 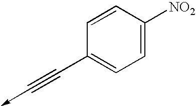 |
| 1477 1478 1479 | rac −+ | 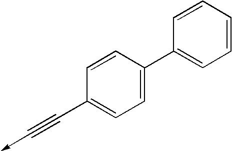 |
| 1480 1481 1482 | rac −+ | 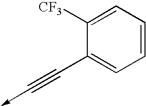 |
| 1483 1484 1485 | rac −+ | 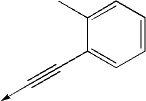 |
| 1486 1487 1488 | rac −+ | 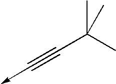 |
| 1489 1490 1491 | rac −+ | 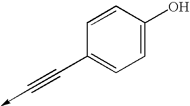 |

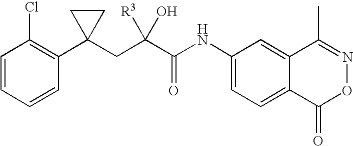

| | | |
|---|---|---|
| 1492 1493 1494 | rac −+ | 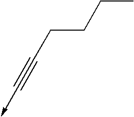 |
| 1495 1496 1497 | rac −+ | 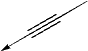 |
| 1498 1499 1500 | rac −+ | 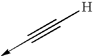 |
| 1501 1502 1503 | rac −+ | 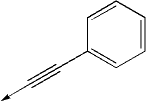 |
| 1504 1505 1506 | rac −+ | 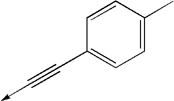 |
| 1507 1508 1509 | rac −+ | 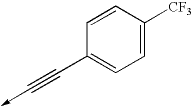 |
| 1510 1511 1512 | rac −+ | 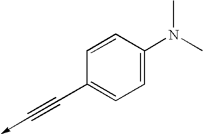 |
| 1513 1514 1515 | rac −+ | 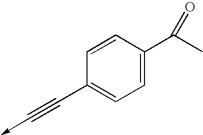 |
| 1516 1517 1518 | rac −+ | 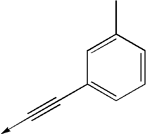 |
| 1519 1520 1521 | rac −+ | 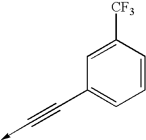 |
| 1522 1523 1524 | rac −+ | 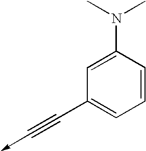 |
| 1525 1526 1527 | rac −+ | 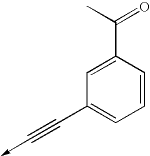 |
| 1528 1529 1530 | rac −+ | 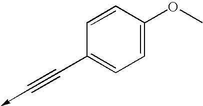 |
| 1531 1532 1533 | rac −+ | 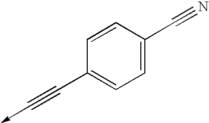 |
| 1534 1535 1536 | rac −+ | 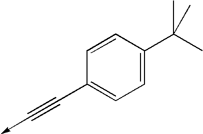 |
| 1537 1538 1539 | rac −+ | 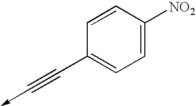 |
| 1540 1541 1542 | rac −+ | 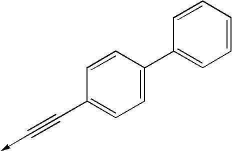 |
| 1543 1544 1545 | rac −+ | 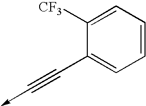 |
| 1546 1547 1548 | rac −+ | 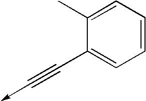 |
| 1549 1550 1551 | rac −+ | 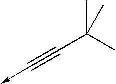 |
| 1552 1553 1554 | rac −+ | 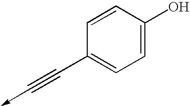 |

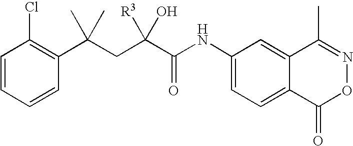

| | | |
|---|---|---|
| 1555 1556 1557 | rac −+ | 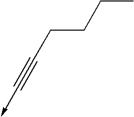 |
| 1558 1559 1560 | rac −+ | 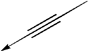 |
| 1561 1562 1563 | rac −+ | 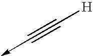 |
| 1564 1565 1566 | rac −+ | 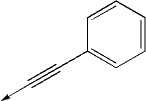 |
| 1567 1568 1569 | rac −+ | 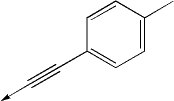 |
| 1570 1571 1572 | rac −+ | 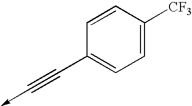 |
| 1573 1574 1575 | rac −+ | 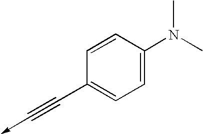 |
| 1576 1577 1578 | rac −+ | 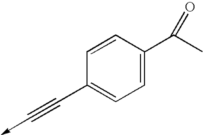 |
| 1579 1580 1581 | rac −+ | 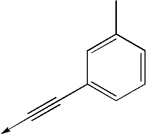 |
| 1582 1583 1584 | rac −+ | 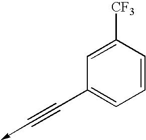 |
| 1585 1586 1587 | rac −+ | 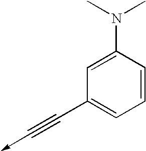 |
| 1588 1589 1590 | rac −+ | 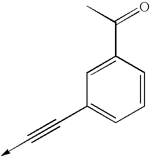 |
| 1591 1592 1593 | rac −+ | 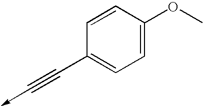 |
| 1594 1595 1596 | rac −+ | 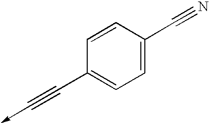 |
| 1597 1598 1599 | rac −+ | 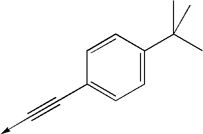 |
| 1600 1601 1602 | rac −+ | 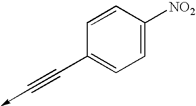 |
| 1603 1604 1605 | rac −+ | 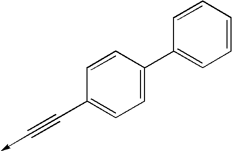 |
| 1606 1607 1608 | rac −+ | 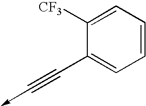 |
| 1609 1610 1611 | rac −+ | 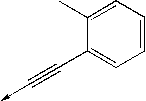 |
| 1612 1613 1614 | rac −+ | 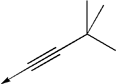 |
| 1615 1616 1617 | rac −+ | 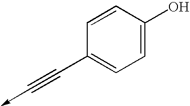 |

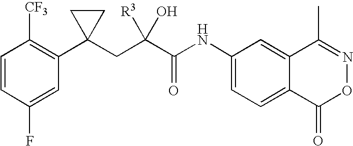

| | | |
|---|---|---|
| 1618 1619 1620 | rac −+ | 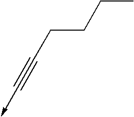 |
| 1621 1622 1623 | rac −+ | 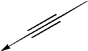 |
| 1624 1625 1626 | rac −+ | 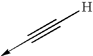 |
| 1627 1628 1629 | rac −+ | 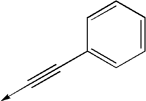 |
| 1630 1631 1632 | rac −+ | 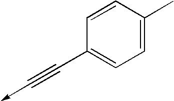 |
| 1633 1634 1635 | rac −+ | 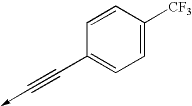 |
| 1636 1637 1638 | rac −+ | 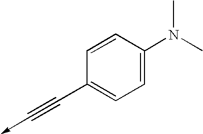 |
| 1639 1640 1641 | rac −+ | 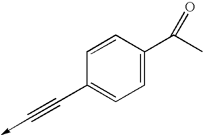 |
| 1642 1643 1644 | rac −+ | 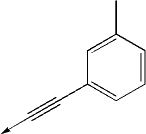 |
| 1645 1646 1647 | rac −+ | 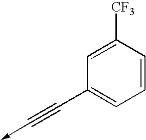 |
| 1648 1649 1650 | rac −+ | 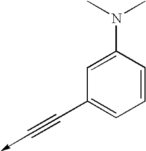 |
| 1651 1652 1653 | rac −+ | 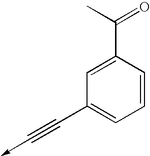 |
| 1654 1655 1656 | rac −+ | 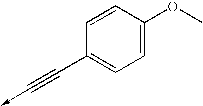 |
| 1657 1658 1659 | rac −+ | 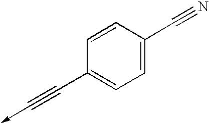 |
| 1660 1661 1662 | rac −+ | 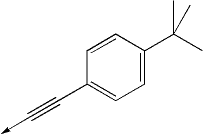 |
| 1663 1664 1665 | rac −+ | 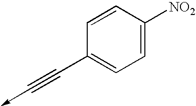 |
| 1666 1667 1668 | rac −+ | 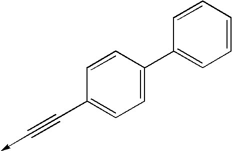 |
| 1669 1670 1671 | rac −+ | 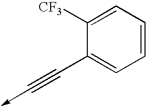 |
| 1672 1673 1674 | rac −+ | 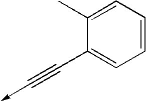 |
| 1675 1676 1677 | rac −+ | 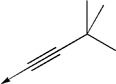 |
| 1678 1679 1680 | rac −+ | 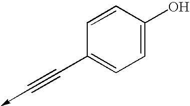 |

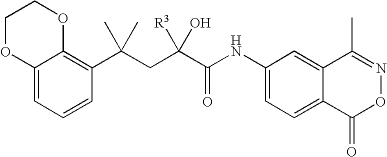

| | | |
|---|---|---|
| 1681 1682 1683 | rac −+ | 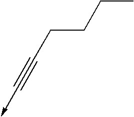 |
| 1684 1685 1686 | rac −+ | 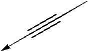 |
| 1687 1688 1689 | rac −+ | 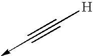 |
| 1690 1691 1692 | rac −+ | 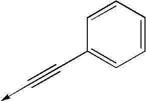 |
| 1693 1694 1695 | rac −+ | 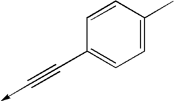 |
| 1696 1697 1698 | rac −+ | 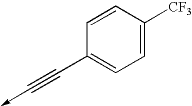 |
| 1699 1700 1701 | rac −+ | 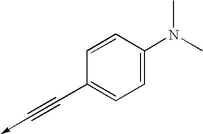 |
| 1702 1703 1704 | rac −+ | 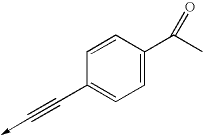 |
| 1705 1706 1707 | rac −+ | 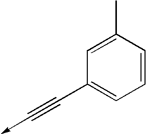 |
| 1708 1709 1710 | rac −+ | 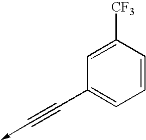 |
| 1711 1712 1713 | rac −+ | 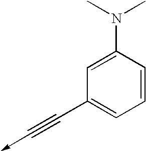 |
| 1714 1715 1716 | rac −+ | 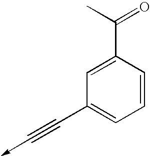 |
| 1717 1718 1719 | rac −+ | 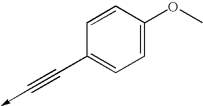 |
| 1720 1721 1722 | rac −+ | 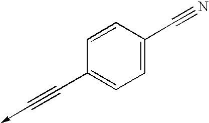 |
| 1723 1724 1725 | rac −+ | 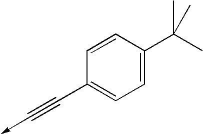 |
| 1726 1727 1728 | rac −+ | 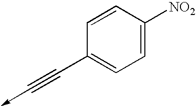 |
| 1729 1730 1731 | rac −+ | 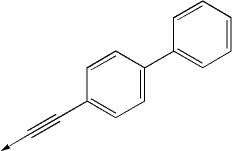 |
| 1732 1733 1734 | rac −+ | 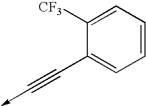 |
| 1735 1736 1737 | rac −+ | 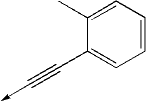 |
| 1738 1739 1740 | rac −+ | 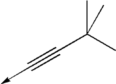 |
| 1741 1742 1743 | rac −+ | 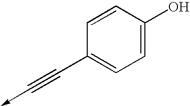 |

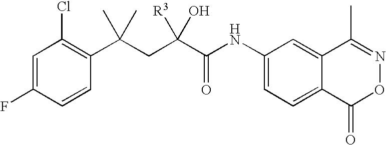

| | | |
|---|---|---|
| 1744 1745 1746 | rac −+ | 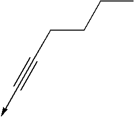 |
| 1747 1748 1749 | rac −+ | 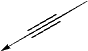 |
| 1750 1751 1752 | rac −+ | 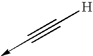 |
| 1753 1754 1755 | rac −+ | 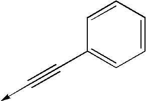 |
| 1756 1757 1758 | rac −+ | 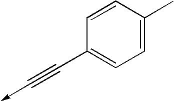 |
| 1759 1760 1761 | rac −+ | 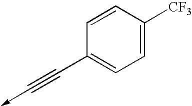 |
| 1762 1763 1764 | rac −+ | 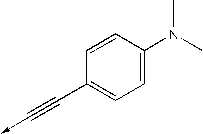 |
| 1765 1766 1767 | rac −+ | 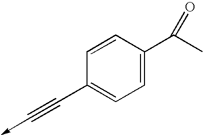 |
| 1768 1769 1770 | rac −+ | 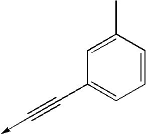 |
| 1771 1772 1773 | rac −+ | 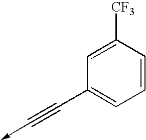 |
| 1774 1775 1776 | rac −+ | 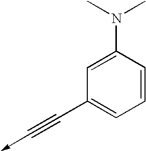 |
| 1777 1778 1779 | rac −+ | 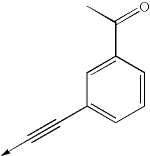 |
| 1780 1781 1782 | rac −+ | 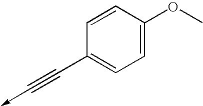 |
| 1783 1784 1785 | rac −+ | 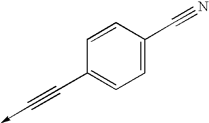 |
| 1786 1787 1788 | rac −+ | 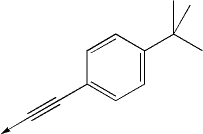 |
| 1789 1790 1791 | rac −+ | 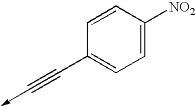 |
| 1792 1793 1794 | rac −+ | 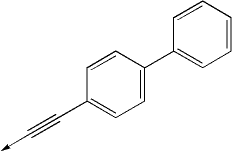 |
| 1795 1796 1797 | rac −+ | 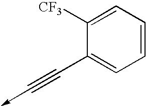 |
| 1798 1799 1800 | rac −+ | 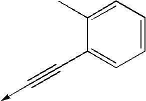 |
| 1801 1802 1803 | rac −+ | 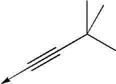 |
| 1804 1805 1806 | rac −+ | 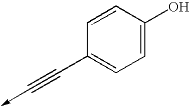 |
| 1807 1808 1809 | rac −+ | 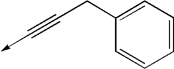 |
| 1810 1811 1812 | rac −+ | 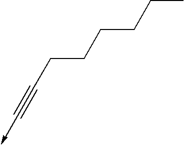 |

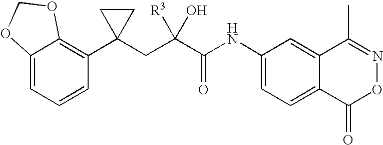

| | | |
|---|---|---|
| 1813 1814 1815 | rac −+ | 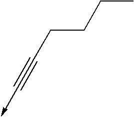 |
| 1816 1817 1818 | rac −+ | 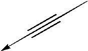 |
| 1819 1820 1821 | rac −+ | 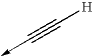 |
| 1822 1823 1824 | rac −+ | 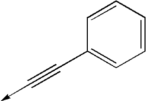 |
| 1825 1826 1827 | rac −+ | 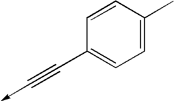 |
| 1828 1829 1830 | rac −+ | 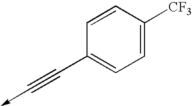 |
| 1831 1832 1833 | rac −+ | 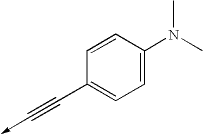 |
| 1834 1835 1836 | rac −+ | 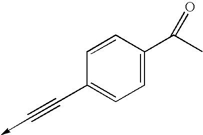 |
| 1837 1838 1839 | rac −+ | 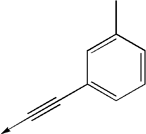 |
| 1840 1841 1842 | rac −+ | 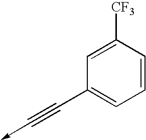 |
| 1843 1844 1845 | rac −+ | 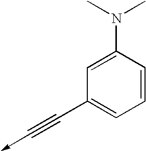 |
| 1846 1847 1848 | rac −+ | 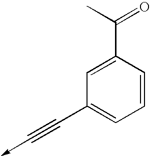 |
| 1849 1850 1851 | rac −+ | 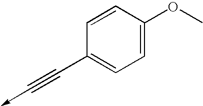 |
| 1852 1853 1854 | rac −+ | 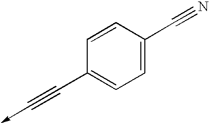 |
| 1855 1856 1857 | rac −+ | 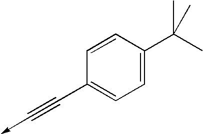 |
| 1858 1859 1860 | rac −+ | 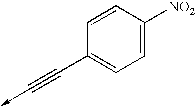 |
| 1861 1862 1863 | rac −+ | 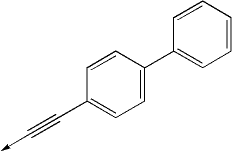 |
| 1864 1865 1866 | rac −+ | 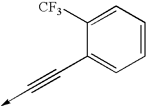 |
| 1867 1868 1869 | rac −+ | 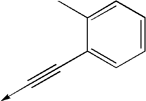 |
| 1870 1871 1872 | rac −+ | 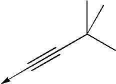 |
| 1873 1874 1875 | rac −+ | 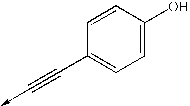 |

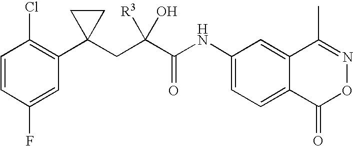

| | | |
|---|---|---|
| 1876 1877 1878 | rac −+ | 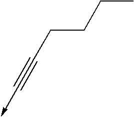 |
| 1879 1880 1881 | rac −+ | 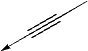 |
| 1882 1883 1884 | rac −+ | 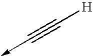 |
| 1885 1886 1887 | rac −+ | 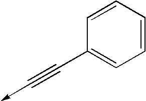 |
| 1888 1889 1890 | rac −+ | 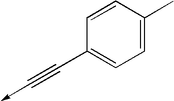 |
| 1891 1892 1893 | rac −+ | 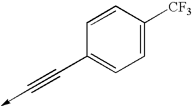 |
| 1894 1895 1896 | rac −+ | 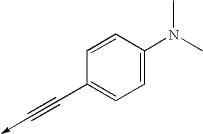 |
| 1897 1898 1899 | rac −+ | 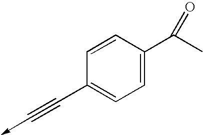 |
| 1900 1901 1902 | rac −+ | 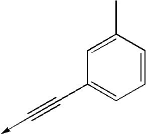 |
| 1903 1904 1905 | rac −+ | 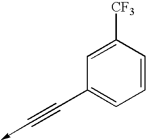 |
| 1906 1907 1908 | rac −+ | 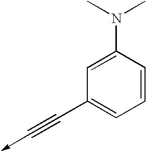 |
| 1909 1910 1911 | rac −+ | 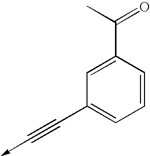 |
| 1912 1913 1914 | rac −+ | 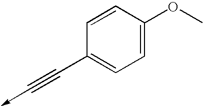 |
| 1915 1916 1917 | rac −+ | 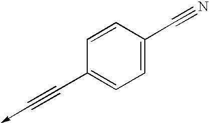 |
| 1918 1919 1920 | rac −+ | 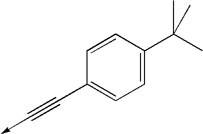 |
| 1921 1922 1923 | rac −+ | 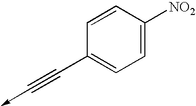 |
| 1924 1925 1926 | rac −+ | 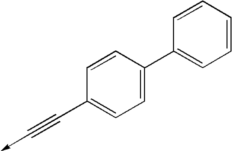 |
| 1927 1928 1929 | rac −+ | 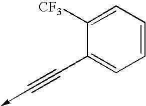 |
| 1930 1931 1932 | rac −+ | 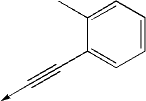 |
| 1933 1934 1935 | rac −+ | 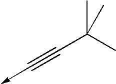 |
| 1936 1937 1938 | rac −+ | 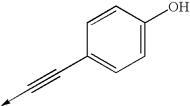 |

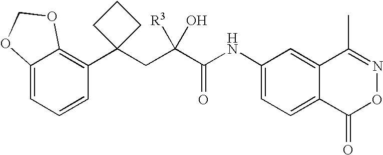

| | | |
|---|---|---|
| 1939 1940 1941 | rac −+ | 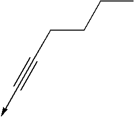 |
| 1942 1943 1944 | rac −+ | 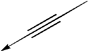 |
| 1945 1946 1947 | rac −+ | 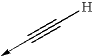 |
| 1948 1949 1950 | rac −+ | 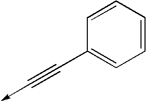 |
| 1951 1952 1953 | rac −+ | 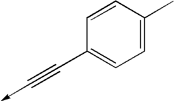 |
| 1954 1955 1956 | rac −+ | 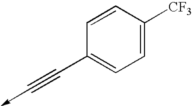 |
| 1957 1958 1959 | rac −+ | 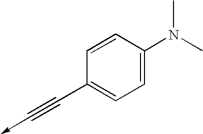 |
| 1960 1961 1962 | rac −+ | 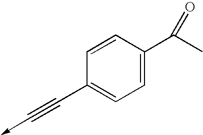 |
| 1963 1964 1965 | rac −+ | 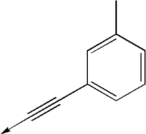 |
| 1966 1967 1968 | rac −+ | 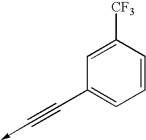 |
| 1969 1970 1971 | rac −+ | 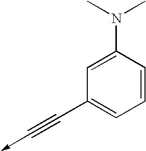 |
| 1972 1973 1974 | rac −+ | 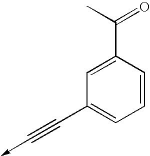 |
| 1975 1976 1977 | rac −+ | 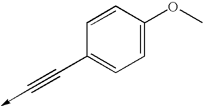 |
| 1978 1979 1980 | rac −+ | 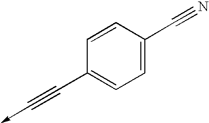 |
| 1981 1982 1983 | rac −+ | 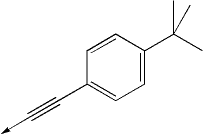 |
| 1984 1985 1986 | rac −+ | 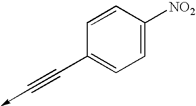 |
| 1987 1988 1989 | rac −+ | 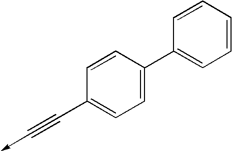 |
| 1990 1991 1992 | rac −+ | 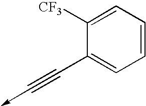 |
| 1993 1994 1995 | rac −+ | 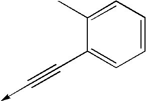 |
| 1996 1997 1998 | rac −+ | 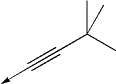 |
| 1999 2000 2001 | rac −+ | 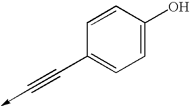 |

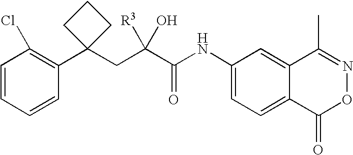

| | | |
|---|---|---|
| 2002 2003 2004 | rac −+ | 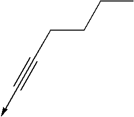 |
| 2005 2006 2007 | rac −+ | 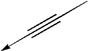 |
| 2008 2009 2010 | rac −+ | 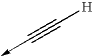 |
| 2011 2012 2013 | rac −+ | 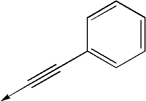 |
| 2014 2015 2016 | rac −+ | 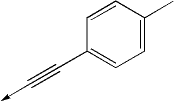 |
| 2017 2018 2019 | rac −+ | 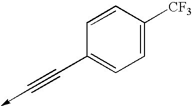 |
| 2020 2021 2022 | rac −+ | 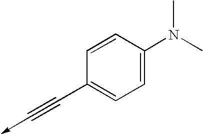 |
| 2023 2024 2025 | rac −+ | 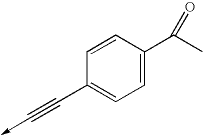 |
| 2026 2027 2028 | rac −+ | 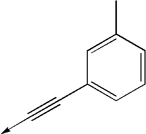 |
| 2029 2030 2031 | rac −+ | 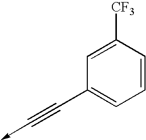 |
| 2032 2033 2034 | rac −+ | 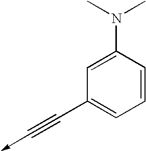 |
| 2035 2036 2037 | rac −+ | 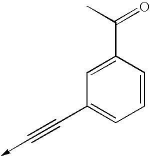 |
| 2038 2039 2040 | rac −+ | 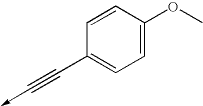 |
| 2041 2042 2043 | rac −+ | 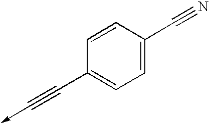 |
| 2044 2045 2046 | rac −+ | 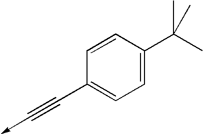 |
| 2047 2048 2049 | rac −+ | 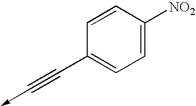 |
| 2050 2051 2052 | rac −+ | 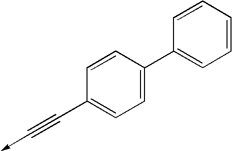 |
| 2053 2054 2055 | rac −+ | 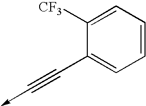 |
| 2056 2057 2058 | rac −+ | 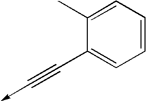 |
| 2059 2060 2061 | rac −+ | 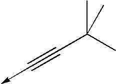 |
| 2062 2063 2064 | rac −+ | 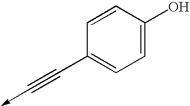 |

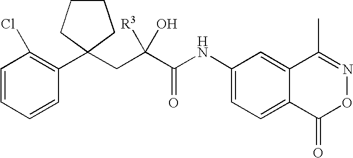

| | | |
|---|---|---|
| 2065 2066 2067 | rac −+ | 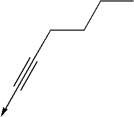 |
| 2068 2069 2070 | rac −+ | 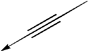 |
| 2071 2072 2073 | rac −+ | 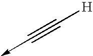 |
| 2074 2075 2076 | rac −+ | 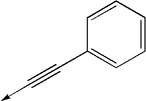 |
| 2077 2078 2079 | rac −+ | 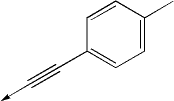 |
| 2080 2081 2082 | rac −+ | 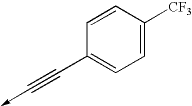 |
| 2083 2084 2085 | rac −+ | 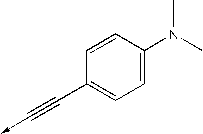 |
| 2086 2087 2088 | rac −+ | 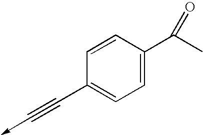 |
| 2089 2090 2091 | rac −+ | 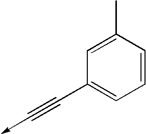 |
| 2092 2093 2094 | rac −+ | 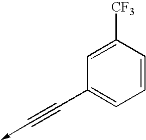 |
| 2095 2096 2097 | rac −+ | 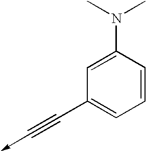 |
| 2098 2099 2100 | rac −+ | 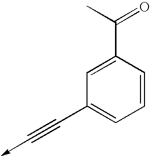 |
| 2101 2102 2103 | rac −+ | 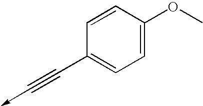 |
| 2104 2105 2106 | rac −+ | 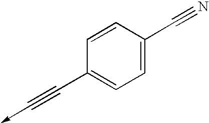 |
| 2107 2108 2109 | rac −+ | 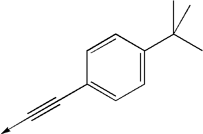 |
| 2110 2111 2112 | rac −+ | 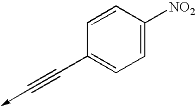 |
| 2113 2114 2115 | rac −+ | 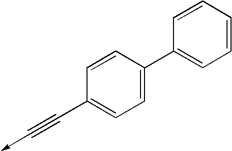 |
| 2116 2117 2118 | rac −+ | 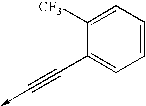 |
| 2119 2120 2121 | rac −+ | 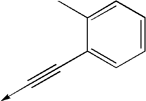 |
| 2122 2123 2124 | rac −+ | 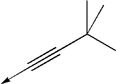 |
| 2125 2126 2127 | rac −+ | 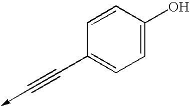 |

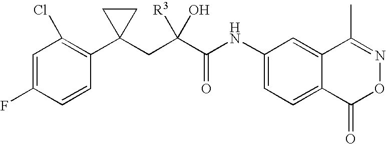

| | | |
|---|---|---|
| 2128 2129 2130 | rac −+ | 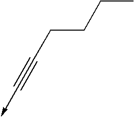 |
| 2131 2132 2133 | rac −+ | 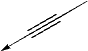 |
| 2134 2135 2136 | rac −+ | 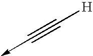 |
| 2137 2138 2139 | rac −+ | 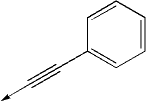 |
| 2140 2141 2142 | rac −+ | 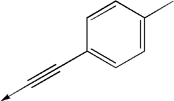 |
| 2143 2144 2145 | rac −+ | 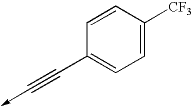 |
| 2146 2147 2148 | rac −+ | 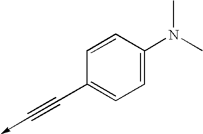 |
| 2149 2150 2151 | rac −+ | 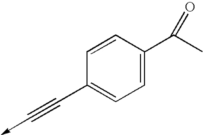 |
| 2152 2153 2154 | rac −+ | 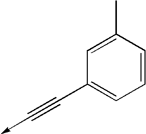 |
| 2155 2156 2157 | rac −+ | 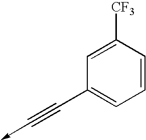 |
| 2158 2159 2160 | rac −+ | 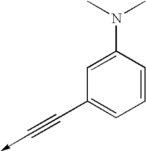 |
| 2161 2163 2163 | rac −+ | 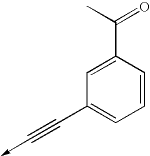 |
| 2164 2165 2166 | rac −+ | 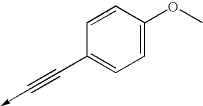 |
| 2167 2168 2169 | rac −+ | 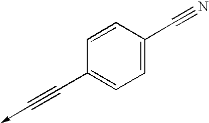 |
| 2170 2171 2172 | rac −+ | 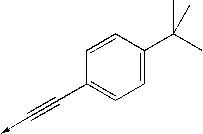 |
| 2173 2174 2175 | rac −+ | 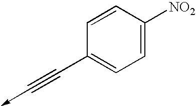 |
| 2176 2177 2178 | rac −+ | 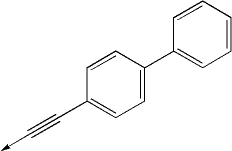 |
| 2179 2180 2181 | rac −+ | 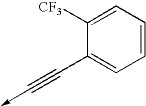 |
| 2182 2183 2184 | rac −+ | 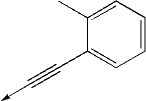 |
| 2185 2186 2187 | rac −+ | 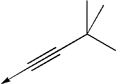 |
| 2188 2189 2190 | rac −+ | 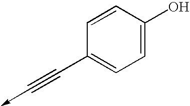 |

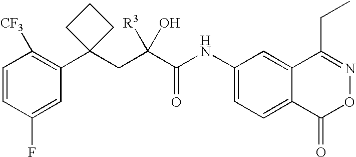

| | | |
|---|---|---|
| 2191 2192 2193 | rac −+ | 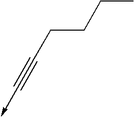 |
| 2194 2195 2196 | rac −+ | 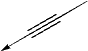 |
| 2197 2198 2199 | rac −+ | 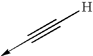 |
| 2200 2201 2202 | rac −+ | 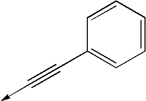 |
| 2203 2204 2205 | rac −+ | 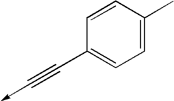 |
| 2206 2207 2208 | rac −+ | 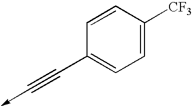 |
| 2209 2210 2211 | rac −+ | 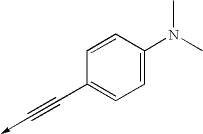 |
| 2212 2213 2214 | rac −+ | 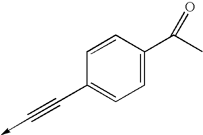 |
| 2215 2216 2217 | rac −+ | 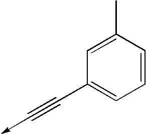 |
| 2218 2219 2220 | rac −+ | 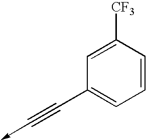 |
| 2221 2222 2223 | rac −+ | 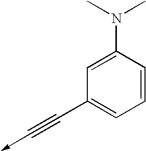 |
| 2224 2225 2226 | rac −+ | 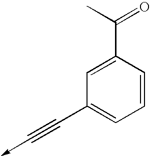 |
| 2227 2228 2229 | rac −+ | 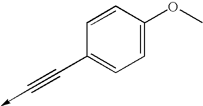 |
| 2230 2231 2232 | rac −+ | 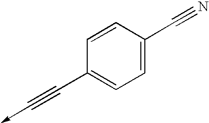 |
| 2233 2234 2235 | rac −+ | 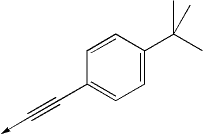 |
| 2236 2237 2238 | rac −+ | 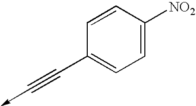 |
| 2239 2240 2241 | rac −+ | 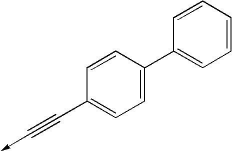 |
| 2242 2243 2244 | rac −+ | 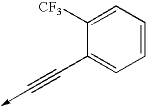 |
| 2245 2246 2247 | rac −+ | 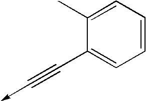 |
| 2248 2249 2250 | rac −+ | 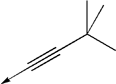 |
| 2251 2252 2253 | rac −+ | 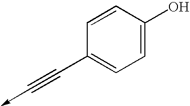 |

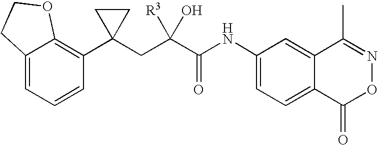

| | | |
|---|---|---|
| 2254 2255 2256 | rac −+ | 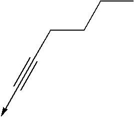 |
| 2257 2258 2259 | rac −+ | 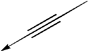 |
| 2260 2261 2262 | rac −+ | 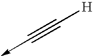 |
| 2263 2264 2265 | rac −+ | 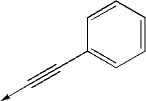 |
| 2266 2267 2268 | rac −+ | 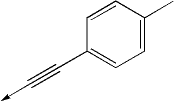 |
| 2269 2270 2271 | rac −+ | 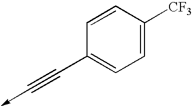 |
| 2272 2273 2274 | rac −+ | 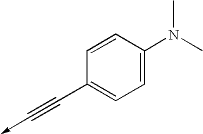 |
| 2273 2276 2277 | rac −+ | 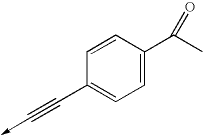 |
| 2278 2279 2280 | rac −+ | 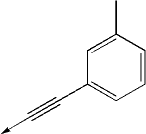 |
| 2281 2282 2283 | rac −+ | 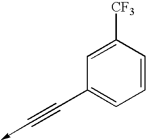 |
| 2284 2285 2286 | rac −+ | 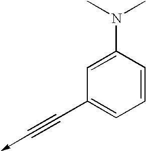 |
| 2287 2288 2289 | rac −+ | 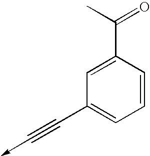 |
| 2290 2291 2292 | rac −+ | 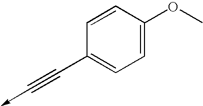 |
| 2293 2294 2295 | rac −+ | 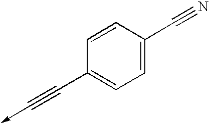 |
| 2296 2297 2298 | rac −+ | 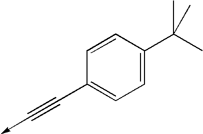 |
| 2299 2300 2301 | rac −+ | 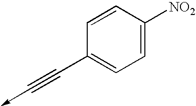 |
| 2302 2303 2304 | rac −+ | 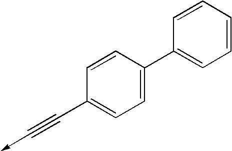 |
| 2305 2306 2307 | rac −+ | 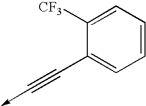 |
| 2308 2309 2310 | rac −+ | 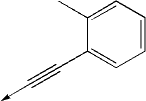 |
| 2311 2312 2313 | rac −+ | 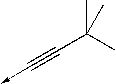 |
| 2314 2315 2316 | rac −+ | 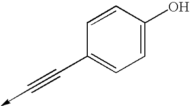 |

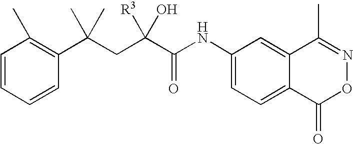

| | | |
|---|---|---|
| 2317 2318 2319 | rac −+ | 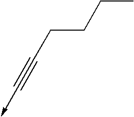 |
| 2320 2321 2322 | rac −+ | 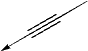 |
| 2323 2324 2325 | rac −+ | 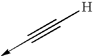 |
| 2326 2327 2328 | rac −+ | 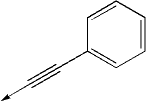 |
| 2329 2330 2331 | rac −+ | 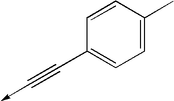 |
| 2332 2333 2334 | rac −+ | 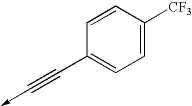 |
| 2335 2336 2337 | rac −+ | 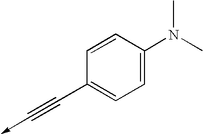 |
| 2338 2339 2340 | rac −+ | 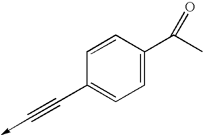 |
| 2341 2342 2343 | rac −+ | 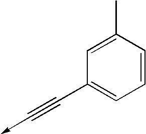 |
| 2344 2345 2346 | rac −+ | 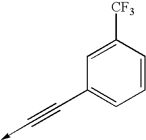 |
| 2347 2348 2349 | rac −+ | 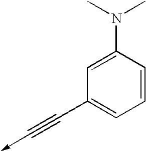 |
| 2350 2351 2652 | rac −+ | 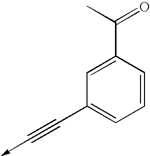 |
| 2353 2354 2355 | rac −+ | 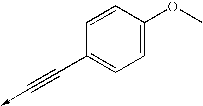 |
| 2356 2357 2358 | rac −+ | 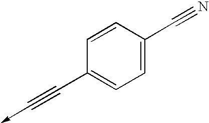 |
| 2359 2360 2361 | rac −+ | 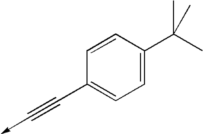 |
| 2362 2363 2364 | rac −+ | 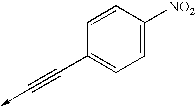 |
| 2365 2366 2367 | rac −+ | 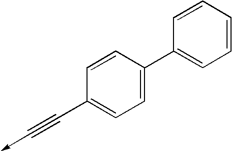 |
| 2368 2369 2370 | rac −+ | 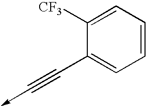 |
| 2371 2372 2373 | rac −+ | 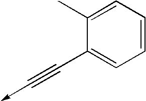 |
| 2374 2375 2376 | rac −+ | 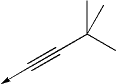 |
| 2377 2378 2379 | rac −+ | 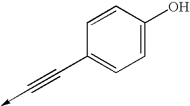 |

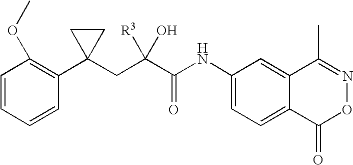

| | | |
|---|---|---|
| 2380 2381 2382 | rac −+ | 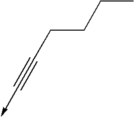 |
| 2383 2384 2385 | rac −+ | 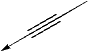 |
| 2386 2387 2388 | rac −+ | 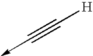 |
| 2389 2390 2391 | rac −+ | 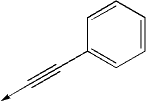 |
| 2392 2393 2394 | rac −+ | 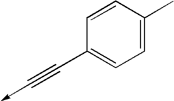 |
| 2395 2396 2397 | rac −+ | 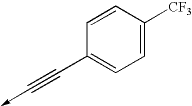 |
| 2398 2399 2400 | rac −+ | 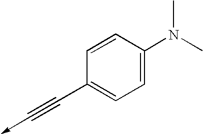 |
| 2401 2402 2403 | rac −+ | 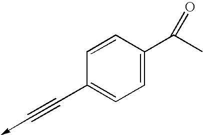 |
| 2404 2405 2406 | rac −+ | 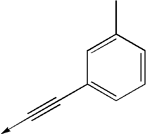 |
| 2407 2408 2409 | rac −+ | 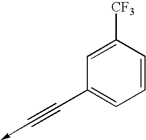 |
| 2410 2411 2412 | rac −+ | 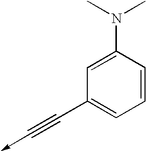 |
| 2413 2414 2415 | rac −+ | 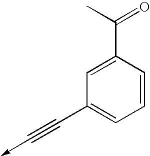 |
| 2416 2417 2418 | rac −+ | 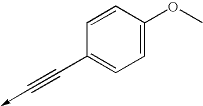 |
| 2419 2420 2421 | rac −+ | 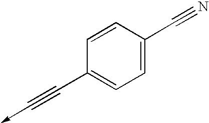 |
| 2422 2423 2424 | rac −+ | 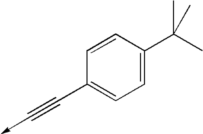 |
| 2425 2426 2427 | rac −+ | 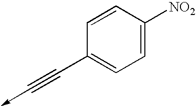 |
| 2428 2429 2430 | rac −+ | 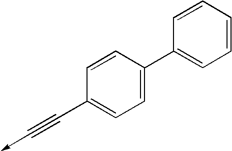 |
| 2431 2432 2433 | rac −+ | 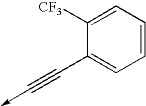 |
| 2434 2435 2436 | rac −+ | 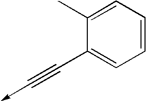 |
| 2437 2438 2439 | rac −+ | 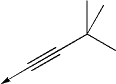 |
| 2440 2441 2442 | rac −+ | 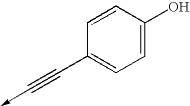 |
| 2443 2444 2445 | rac −+ | 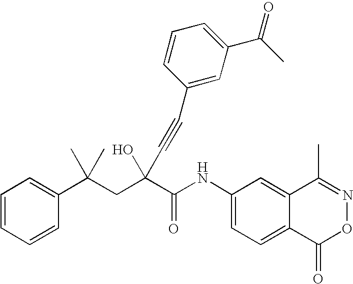 |
| 2446 2447 2448 | rac −+ | 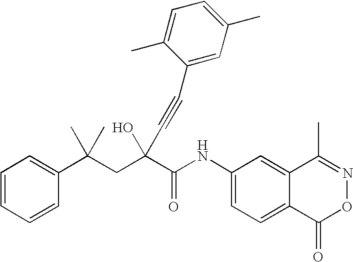 |
| 2449 2450 2451 | rac −+ | 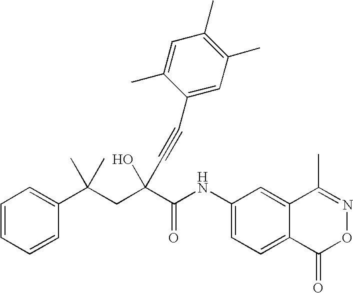 |
| 2452 2453 2454 | rac −+ | 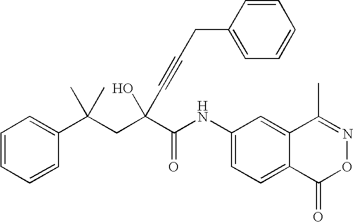 |
| 2455 2456 2457 | rac −+ | 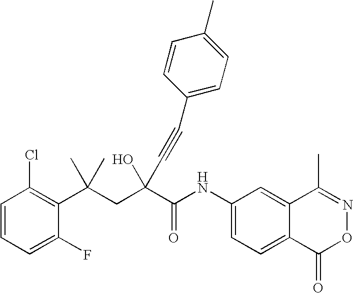 |
| 2458 2459 2460 | rac −+ | 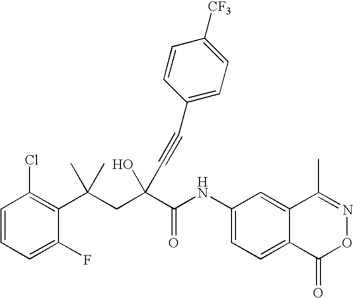 |
| 2461 2462 2463 | rac −+ | 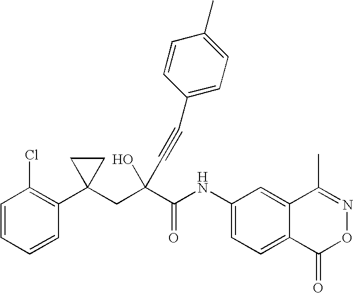 |
| 2464 2465 2466 | rac −+ | 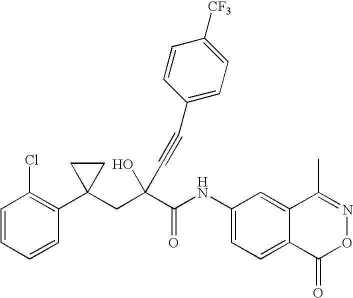 |
| 2467 2468 2469 | rac −+ | 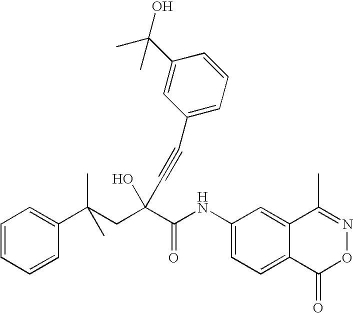 |
| 2470 2471 2472 | rac −+ | 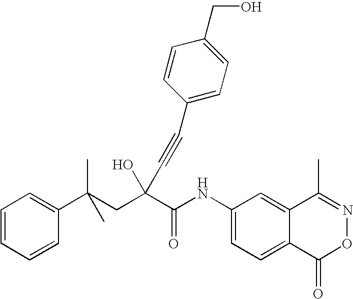 |
| 2473 2474 2475 | rac −+ | 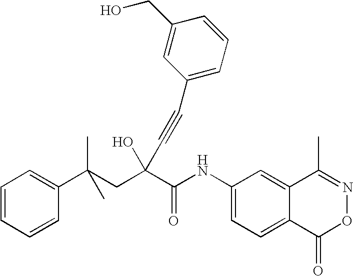 |
| 2476 2477 2478 | rac −+ | 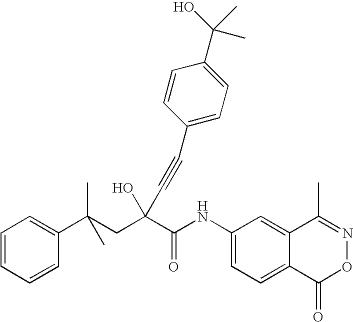 |

Biological Characterization of the Compounds According to the Invention

Progesterone receptor modulators can be identified with the aid of simple methods, test programmes known to the skilled person. It is possible for this purpose for example to incubate a compound to be tested together with a progestogen in a test system for progesterone receptors and to check whether the effect mediated by progesterone is altered in the presence of the modulator in this test system.

The substances according to the invention of the general formula I were tested in the following models:

Progesterone Receptor-Binding Assay

Measurement of the Receptor Binding Affinity:

The receptor binding affinity was determined by competitive binding of a specifically binding ³H-labelled hormone (tracer) and of the compound to be tested on receptors in the cytosol from animal target organs. The aim in this case was receptor saturation and reaction equilibrium.

The tracer and increasing concentrations of the compound to be tested (competitor) were coincubated at 0-4° C. for 18 h with the receptor-containing cytosol fraction. After removal of unbound tracer with carbon-dextran suspension, the receptor-bound tracer content was measured for each concentration, and the IC₅₀ was determined from the concentration series. The relative molar binding affinity (RBA) was calculated as ratio of the IC₅₀ values for reference substance and compound to be tested (×100%) (RBA of the reference substance=100%).

The following incubation conditions were chosen for the receptor types:

Progesterone Receptor:

Uterus cytosol of the estradiol-primed rabbit, homogenized in TED buffer (20 mMTris/HCl, pH 7.4; 1 mM ethylenediaminetetraacetate, 2 mM dithiothreitol) with 250 mM sucrose; stored at −30° C. Tracer: ³H-ORG 2058, 5 nM; reference substance: progesterone.

Glucocorticoid Receptor:

Thymus cytosol from the adrenalectomized rat, thymi stored at −30° C.; buffer: TED. Tracer: ³H-dexamethasone, 20 nM; reference substance: dexamethasone.

The relative receptor binding affinities (RBA values) for the compounds according to the invention of the general formula (I) on the progesterone receptor are between 3 and 100% relative to progesterone. The RBA values at the glucocorticoid receptor are in the range from 3 to 30% relative to dexamethasone.

The compounds according to the invention accordingly have a high affinity for the progesterone receptor, but only a low affinity for the glucocorticoid receptor.

Antagonism at the PR-B Progesterone Receptor

The transactivation assay is carried out as described in WO 02/054064.

Agonism on the PR-B Progesterone Receptor

The transactivation assay is carried out as described in Fuhrmann et al. (Fuhrmann U., Hess-Stump H., Cleve A., Neef G., Schwede W., Hoffmann J., Fritzemeier K.-H., Chwalisz K., Journal of Medicinal Chem, 43, 26, 2000, 5010-5016).

| | |
|---|---|
| Antagonistic Activity | Agonistic Acticity |

| No. | IC₅₀ [nM] | Efficacy [%] | EC₅₀ [nM] | Efficacy [%] |
|---|---|---|---|---|
| 5 | 0.2 | 86 | 0.2 | 10 |
| 14 | 6 | 53 | 7 | 35 |
| 16 | 0.7 | 82 | 0.5 | 13 |
| 17b | 0.03 | 88 | n.b. | 7 |
| 18 | 0.2 | 89 | n.b. | 8 |
| 24 | 2 | 100 | | 0 |
| 35 | 2 | 100 | | 0 |

Dosage

The progesterone receptor modulators can be administered orally, enterally, parenterally or transdermally for the use according to the invention.

Satisfactory results are generally to be expected in the treatment of the indications mentioned hereinbefore when the daily doses cover a range from 1 μg to 500 mg of the compound according to the invention.

Suitable dosages of the compounds according to the invention in humans for the treatment of endometriosis, of leiomyomas of the uterus and dysfunctional bleeding and for use in fertility control and for hormone replacement therapy are from 50 μg to 500 mg per day, depending on the age and constitution of the patient, it being possible to administer the necessary daily dose by single or multiple administration.

The dosage range for the compounds according to the invention for the treatment of breast carcinomas is 10 mg to 1000 mg per day.

The pharmaceutical products based on the novel compounds are formulated in a manner known per se by processing the active ingredient with the carrier substances, fillers, substances influencing disintegration, binders, humectants, lubricants, absorbents, diluents, masking flavours, colorants, etc. which are used in pharmaceutical technology, and converting into the desired administration form. Reference should be made in this connection to Remington's Pharmaceutical Sciences, 15^{th} ed. Mack Publishing Company, Easton, Pa. (1980).

Suitable for oral administration are in particular tablets, film-coated tablets, sugar-coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injection or suspensions and corresponding depot preparations can be used for intramuscular injection.

For rectal administration, the novel compounds can be used in the form of suppositories, capsules, solutions (e.g. in the form of enemas) and ointments, both for systemic and for local therapy.

Furthermore, compositions for vaginal use may also be mentioned as preparation.

For pulmonary administration of the novel compounds, they can be used in the form of aerosols and inhalants.

Patches are possible for transdermal administration, and formulations in gels, ointments, fatty ointments, creams, pastes, dusting powders, milk and tinctures are possible for topical application. The dosage of the compounds of the general formula I in these preparations should be 0.01%-20% in order to achieve an adequate pharmacological effect.

Corresponding tablets can be obtained for example by mixing active ingredient with known excipients, for example inert diluents such as dextrose, sugar, sorbitol, mannitol, polyvinylpyrrolidone, disintegrants such as maize starch or alginic acid, binders such as starch or gelatin, lubricants such as magnesium stearate or talc and/or means to achieve a depot effect such as carboxypolymethylene, carboxymethylcellulose, cellulose acetate phthalate or polyvinyl acetate. The tablets may also consist of a plurality of layers.

Correspondingly, coated tablets can be produced by coating cores produced in analogy to the tablets with compositions normally used in tablet coatings, for example polyvinylpyrrolidone or shellac, gum arabic, talc, titanium oxide or sugar. The tablet covering may in this case also consist of a plurality of layers, it being possible to use the excipients mentioned above for tablets.

Solutions or suspensions of the compounds according to the invention of the general formula I may additionally comprise taste-improving agents such as saccharin, cyclamate or sugar, and, for example, flavourings such as vanillin or orange extract. They may additionally comprise suspending excipients such as sodium carboxymethylcellulose or preservatives such as p-hydroxybenzoates.

Capsules comprising the compounds of the general formula I can be produced for example by mixing the compound(s) of the general formula I with an inert carrier such as lactose or sorbitol and encapsulating it in gelatin capsules.

Suitable suppositories can, be produced for example by mixing with carriers intended for this purpose, such as neutral fats or polyethylene glycol or derivatives.

The compounds according to the invention of the general formula (I) or their pharmaceutically acceptable salts can be used, because of their antagonistic or partial agonistic activity, for producing a medicament, in particular for the treatment and prophylaxis of gynaecological disorders such as endometriosis, leiomyomas of the uterus, dysfunctional bleeding and dysmenorrhoea. They can furthermore be employed to counteract hormonal irregularities, for inducing menstruation and alone or in combination with prostaglandins and/or oxytocin to induce labour.

The compounds according to the invention of the general formula (I) or their pharmaceutically acceptable salts are furthermore suitable for producing products for female contraception (see also WO 93/23020, WO 93/21927).

The compounds according to the invention or their pharmaceutically acceptable salts can additionally be employed alone or in combination with a selective estrogen receptor modulator (SERM) for female hormone replacement therapy.

In addition, the said compounds have an antiproliferative effect in hormone-dependent tumours. They are therefore suitable for the therapy of hormone-dependent carcinomas such as, for example, for breast, prostate and endometrial carcinomas.

The compounds according to the invention or their pharmaceutically acceptable salts can be employed for the treatment of hormone-dependent carcinomas both in first-line therapy and in second-line therapy, especially after tamoxifen failure.

The compounds according to the invention, having antagonistic or partially agonistic activity, of the general formula (I) or their pharmaceutically acceptable salts can also be used in combination with compounds having antiestrogenic activity (estrogen receptor antagonists or aromatase inhibitors) or selective estrogen receptor modulators (SERM) for producing pharmaceutical products for the treatment of hormone-dependent tumours. The compounds according to the invention can likewise be used in combination with SERMs or an antiestrogen (estrogen receptor antagonist or aromatase inhibitor) for the treatment of endometriosis or of leiomyomas of the uterus. In the treatment of hormone-dependent tumours the progesterone receptor modulator and the antiestrogen (estrogen receptor antagonists or aromatase inhibitors) or the SERM can be provided for simultaneous or else for sequential administration. In the sequential administration, preferably the antiestrogen (estrogen receptor antagonists or aromatase inhibitors) or SERM is administered first and subsequently the progesterone receptor modulator is administered.

Suitable for combination with the non-steroidal progesterone receptor modulators according to the invention in this connection are for example the following antiestrogens (estrogen receptor antagonists or aromatase inhibitors) or SERMs: tamoxifen, 5-(4-{5-[(RS)-(4,4,5,5,5-pentafluoropentyl)sulphinyl]pentyloxy}phenyl)-6-phenyl-8,9-dihydro-7H-benzocyclohepten-2-ol (WO 00/03979), ICI 182 780 (7alpha-[9-(4,4,5,5-pentafluoropentylsulphinyl)nonyl]estra-1,3,5(10)-triene-3,17beta-diol), 11beta-fluoro -7alpha-[5-(methyl{3-[(4,4,5,5,5-pentafluoropentyl)sulphanyl]propyl}amino)pentyl]-estra-1,3,5(10)-triene-3,17beta-diol (WO98/07740), 11beta-fluoro-7alpha-{5-[methyl(7,7,8,8,9,9,10,10,10-nonafluorodecyl)amino]pentyl}estra-1,3,5(10)-triene-3,17-beta-diol (WO 99/33855), 11beta-fluoro-17alpha-methyl-7alpha-{5-[methyl(8,8,9,9,9-pentafluorononyl)amino]pentyl}estra-1,3,5(10)-triene-3,17beta-diol (WO 03/045972), clomifen, raloxifen, and further compounds having antiestrogenic activity, and aromatase inhibitors such as, for example, fadrozole, formestane, letrozole, anastrozole or atamestane.

Finally, the present invention also relates to the use of the compounds of the general formula I, where appropriate together with an antiestrogen or SERM, for producing a medicament.

The present invention further relates to pharmaceutical compositions which comprise at least one compound according to the invention, where appropriate in the form of a pharmaceutically/pharmacologically acceptable salt, without or together with pharmaceutically acceptable excipients and/or carriers.

These pharmaceutical compositions and medicaments may be intended for oral, rectal, vaginal, subcutaneous, percutaneous, intravenous or intramuscular administration. Besides conventional carriers and/or diluents, they comprise at least one compound according to the invention.

The medicaments of the invention are produced with the conventional solid or liquid carriers or diluents and the excipients normally used in pharmaceutical technology appropriate for the desired mode of administration with a suitable dosage in a known manner. The preferred preparations consist of a dosage suitable for oral administration. Examples of such dosage forms are tablets, film-coated tablets, sugar-coated tablets, capsules, pills, powders, solutions or suspensions or else depot forms.

The pharmaceutical compositions comprising at least one of the compounds according to the invention are preferably administered orally.

Also suitable are parenteral preparations such as solutions for injection. Further preparations which may also be mentioned are for example suppositories and compositions for vaginal use.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The following examples serve to illustrate the subject-matter of the invention in more detail without wishing to restrict it thereto.

Preparation of the starting compounds 6-[4-(2-chloro-5-fluorophenyl)-4-methyl-2-oxovaleroylamino]-4-methyl-2,3-benzoxazin-1-one, 6-[4-(2-chloro-4-fluorophenyl)-4-methyl-2-oxovaleroylamino]-4-methyl-2,3-benzoxazin-1-one and 6-{3-[1-(2-chlorophenyl)cyclopentyl]-2-oxopropionylamino}-4-methyl-2,3-benzoxazin-1-one has been described in the patent US 2002/0077356, the compound 6-[4-(2,3-dihydro-7-benzofuranyl)-4-methyl-2-oxopentanoylamino]-4-methyl-2,3-benzoxazinone in U.S. Pat. No. 6,323,199B1 (example 87 therein), the compound 6-(4-methyl-4-phenyl-2-oxovaleroylamino)-4-methyl-2,3-benzoxazin-1-one in the patent WO 199854159 and the compound 6-[3-[1-(2-fluoro-5-trifluoromethylphenyl)cyclopropyl]-2-oxopropionylamino]-4-methyl-2,3-benzoxazin-1-one in the patent WO 200375915.

General Methods

### 1-(Benzo[1,3]dioxol-4-yl)-1-methylethanol

57.2 ml of methyl magnesium chloride solution (3M in THF) were added to 25.5 g of 4-acetylbenzo[1,3]dioxole in 375 ml of THF at RT under argon. The mixture was stirred at RT for 16 h and added to ice/2N hydrochloric acid. It was then extracted with ethyl acetate, and the organic phase was washed with water and brine and dried (Na₂SO₄). 27.89 g of 1-[benzo(1,3)dioxol-4-yl]-1-methylethanol were obtained as a brown oil.

¹H-NMR (CDCl₃, ppm)=1.6 (s, 6H), 5.95 (s, 2H), 6.76 (dd, 1H), 6.82 (t, 1H), 6.91 (dd, 1H)

### 4-(Benzo[1,3]dioxol-4-yl)-4-methyl-2-oxopentanoic acid

47 ml of tin(IV) chloride were added to 9.5 g of 1-(benzo[1,3]dioxol-4-yl)-1-methylethanol and 14.2 g of ethyl 2-trimethylsilyloxyacrylate in 200 ml of dichloromethane at −70° C. After 15 minutes, the solution was added to potassium carbonate solution. After extraction with diethyl ether, the organic phase was washed with water, dried and evaporated.

14.4 g of the ethyl 4-(benzo[1,3]dioxol-4-yl)-4-methyl-2-oxopentanoate obtained in this way were stirred with 150 ml of 1 M sodium hydroxide and 300 ml of methanol at RT for 10 hours. The methanol was then removed in vacuo, and the remaining solution was extracted with diethyl ether. The aqueous phase was acidified with 1 M hydrochloric acid and extracted with diethyl ether. Drying and evaporation resulted in 11.1 g of 4-(benzo[1,3]dioxol-4-yl)-4-methyl-2-oxopentanoic acid as yellowish oil.

MS (ei) m/e: M⁺=251

### 6-[4-(Benzo[1, 3]dioxol-4-yl)-4-methyl-2-oxovaleroylamino]-4-methyl-2,3-benzoxazin-1-one

10 g of 4-(benzo[1,3]dioxol-4-yl)-4-methyl-2-oxopentanoic acid were dissolved in 125 ml of dimethylacetamide and, at −0° C. under argon, 3.5 ml of thionyl chloride were added. After stirring at −3 to +3° C. for 20 minutes, 7.6 g of 6-amino-4-methyl-2,3-benzoxazin-1-one (WO 00/32584) were added. The mixture was stirred at room temperature for 96 hours and, after addition of water, extracted with ethyl acetate, the organic phase was washed with water and dried (Na₂SO₄), and evaporation of the solvent and chromatography of the crude product on silica gel with hexane/ethyl acetate (100:0->60:40) resulted in 6.56 g of 6-[4-(benzo[1,3]dioxol-4-yl)-4-methyl-2-oxovaleroylamino]-4-methyl-2,3-benzoxazin-11-one as a beige solid.

m.p.=165-166° C., MS (ei) m/e: M⁺=409

### SYNTHESIS EXAMPLES

### (−)-6-{2-[2-(2,3-(Methylenedioxy)phenyl)-2-methylpropyl]-2-hydroxyoct-3-ynoyl}-4-methyl-2,3-benzoxazin-1-one 1 and (+)-6-{2-[2-(2,3-(methylenedioxy)phenyl)-2-methylpropyl]-2-hydroxyoct-3-ynoyl}-4-methyl-2,3-benzoxazin-1-one 2

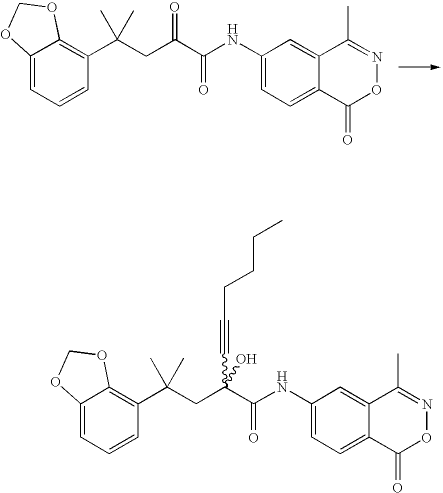

nBuLi (0.7 ml, 1.6M in hexane) was added to a solution of 1-hexyne (0.5 ml) in THF (4 ml) at −78° C. The mixture was stirred at −78° C. for 20 min, 6-[4-(benzo[1,3]dioxol-4-yl)-4-methyl-2-oxovaleroylamino]-4-methyl-2,3-benzoxazin-1-one (192 mg) was added, and the mixture was stirred at −78° C. for 4 h. Water was then added and the mixture was allowed to reach room temp. Extraction with ethyl acetate, washing with saturated sodium chloride solution, drying over sodium sulphate and purification by column chromatography on silica gel resulted in 82 mg of a white foam which was then converted by preparative chiral HPLC (Chiralpak AD 250×10 mm, eluent: acetonitrile/water 55/45 v/v, flow rate 4.7 ml/min, temperature 40° C., retention times: 12.2 min (+)-enantiomer, 15.7 min (−)-enantiomer) into the compounds (−)-6-{2-[2-(2,3-(methylenedioxy)phenyl)-2-methylpropyl]-2-hydroxyoct-3-ynoyl}-4-methyl-2,3-benzoxazin-1-one (Example 1) and (+)-6-{2-[2-(2,3-(methylenedioxy)phenyl)-2-methylpropyl]-2-hydroxyoct-3-ynoyl}-4-methyl-2,3-benzoxazin-1-one (Example 2).

1H-NMR (ppm, CDCl₃, 400 MHz): 0.91 (t, J=7.2 Hz, 3H, CH₃), 1.32-1.49 (m, 4H), 1.55 (s, 3H), 1.58 (s, 3H), 2.17 (t, J=7.2 Hz, 2H), 2.56 (s, 3H, CH₃), 2.59 (d, J=14.4 Hz, 1H), 2.74 (d, J=14.8 Hz, 1H), 2.80 (s, 1H, OH), 5.94-5.96 (m, 2H), 6.46-6.49 (m, 1H), 6.64 (t, J=7.8 Hz, 1H), 7.47-7.49 (m, 1H), 8.25-8.28 (m, 1H), 8.76 (s, 1H, NH). C₂₈H₃₀N₂O₆ (490.6):

### rac-6-{2-[2-(2-Chloro-5-fluorophenyl)-2-methylpropyl]-2,7-dihydroxyhept-3-ynoyl}-4-methyl-2,3-benzoxazin-1-one 3

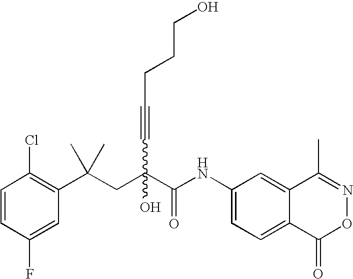

Stage A: Reaction of 5-(tert-butyldimethylsilyloxy)pent-1-yne (531 mg), nBuLi (0.7 ml, 1.6 M in hexane) and 6-[4-(2-chloro-5-fluorophenyl)-4-methyl-2-oxovaleroylamino]-4-methyl-2,3-benzoxazin-1-one (207 mg) at −78° C. as described for Example 1 gave, after column chromatography on silica gel, a colourless oil (86 mg).

Stage B: The resulting oil was stirred in THF (3 ml) at room temp. under argon (3 h). Addition of water, extraction with ethyl acetate and washing with saturated brine were followed by drying with sodium sulphate. Purification by column chromatography on silica gel led to the title compound as a white foam (43 mg).

1H-NMR (ppm, CDCl₃, 400 MHz): 1.58 (s, 3H, Me), 1.59 (s, 3H, Me), 1.71-1.74 (m, 2H, CH₂), 2.2-2.3 (m, 2H), 2.56 (s, 3H, CH₃), 2.75 (d, J=15.2 Hz, 1H, CH), 2.92 (d, J=14.8 Hz, 1H, CH), 3.26 (s, 1H, OH), 3.74-3.78 (m, 2H), 6.67-6.78 (m, 1H), 7.09-7.19 (m, 2H), 7.66-7.69 (m, 2H), 8.20-8.21 (m, 1H), 8.27-8.29 (m, 1H), 8.99 (s, 1H, NH). C₂₆H₂₆ClFN₂O₅ (501.0): LC-MS: m/z=501 [M+H⁺].

### rac-6-{2-[2-(2-Chloro-5-fluorophenyl)-2-methylpropyl]-2-hydroxyoct-3-ynoyl}-4-methyl-2,3-benzoxazin-1-one 4

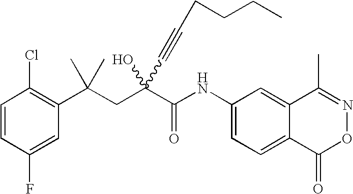

Reaction of 1-hexyne (0.6 ml), nBuLi (0.7 ml, 1.6 M in hexane) and 6-[4-(2-chloro-5-fluorophenyl)-4-methyl-2-oxovaleroylamino]-4-methyl-2,3-benzoxazin-1-one (207 mg) at −78° C. as described for Example 1 gave, after column chromatography on silica gel and preparative thin-layer chromatography a viscous oil (12 mg).

1H-NMR (ppm, CDCl₃, 400 MHz): 0.90 (t, J=7.2 Hz, 3H, Me), 1.32-1.47 (m, 4H), 1.57 (s, 3H, Me), 1.62 (s, 3H, Me), 2.13 (t, J=7.2 Hz, CH₂C≡C), 2.56 (s, 3H, Me), 2.81-2.95 (m, 3H), 6.68-6.71 (m, 1H), 7.11-7.17 (m, 2H), 7.56-7.58 (m, 1H), 8.21 (d, J=2.0 Hz, 1H), 8.29 (d, J=12.6 Hz, 1H), 8.73 (br. s., 1H, NH). C₂₇H₂₈ClFN₂O₄ (499.0): LC-MS: m/z=499 [M+H⁺].

### rac-6-{2-[(2-Chlorophenyl)cyclopentyl]methyl-2-hydroxy-4-phenylbut-3-ynoylamino}-4-methyl-2,3-benzoxazin-1-one 5

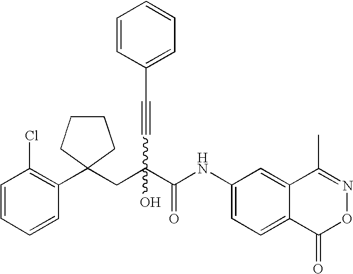

Lithiumphenylacetylide (0.65 ml, 1M in THF) was added to 6-{3-[1-(2-chlorophenyl)cyclopentyl]-2-oxopropionylamino}-4-methyl-2,3-benzoxazin-1-one (110 mg) at −78° C. and allowed to reach room temperature under argon during the night. Working up as described for Example 1 and column chromatography on silica gel resulted in the title compound as a foam (54 mg) after oil-pump drying. ¹H-NMR (ppm, CDCl₃, 400 MHz): 1.59-1.85 (m, 5H), 2.18-2.35 (m, 3H), 2.54 (s, 3H, Me), 2.7-3.09 (3H), 6.94-7.58 (m, 10H), 8.18 (d, J=1.1 Hz), 8.25 (d, J=8.6 Hz, 1H), 8.81 (br. s., 1H, NH). C₃₁H₂₇ClN₂O₄ (526.0): HPLCMS: m/z=526 [M], purity 97%.

### 6-{2-[2-(2,3-Dihydro-7-benzofuranyl)-2-methylpropyl]-2-hydroxy-3-octynoylamino}-4-methyl-2,3-benzoxazin-1-one 6

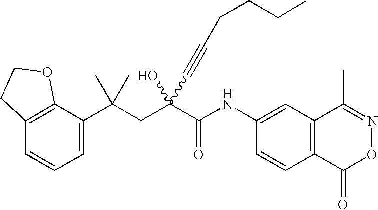

Reaction of 1-hexyne (0.4 ml), nBuLi (0.7 ml, 1.6 M in hexane) and 6-[4-(2,3-dihydro-7-benzofuranyl)-4-methyl-2-oxopentanoylamino]-4-methyl-2,3-benzoxazin-1-one (99.5 mg) at −78° C. in THF (3 ml) as described for Example 1 gave, after column chromatography on silica gel and drying in vacuo, a solidified colourless oil (42 mg). 1H NMR (ppm, CDCl₃, 400 MHz): 0.89 (t, J=7.2 Hz, 3H, Me), 1.35-1.56 (m, 10H), 2.14-2.18 (m, 2H), 2.56 (s, 3H, Me); 2.66 (d, J=14.8 Hz, 1H), 2.73 (d, J=14.8 Hz,

1H), 3.0-3.2 (m, 2H), 3.27 (s, 1H), 4.57 (t, J=9.3 Hz, 2H), 6.75 (t, J=7.5 Hz, 1H), 6.95 (d, J=6.3 Hz, 1H), 7.05 (d, J=7.8 Hz, 1H), 7.50-7.52 (m, 1H), 8.23-8.29 (m, 2H), 8.78 (br. s., NH). C₂₉H₃₂ClN₂O₅ (488): LC-MS: m/z=489 [M+H⁺].

### rac-6-{4-(2-Chloro-4-fluorophenyl)-2-hydroxy-2-[(4-hydroxyphenyl)ethynyl]-4-methylpentanoylamino}-4-methyl-2,3-benzoxazin-1-one 7

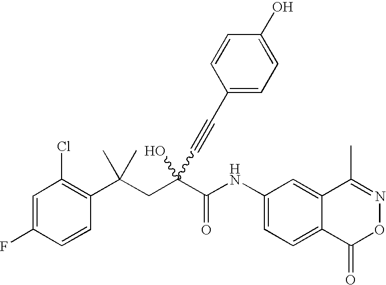

Stage A: a suspension of the compound of Example 10 (57.8 mg), triphenylphosphine (6.8 mg), copper iodide (5 mg), 4-iodophenyl acetate (51 mg), 5 mg of palladium acetate in THF (1 ml) and triethylamine. (3 ml) was reacted in an ultrasonic bath under argon for 1 h. Addition of saturated aqueous ammonium chloride solution was followed by extraction with ethyl acetate and washing with water and brine. Drying with sodium sulphate was followed by concentration and purification by column chromatography on silica gel. A white solid (46.7 mg) was obtained. Stage B: A suspension of the compound from stage A (46.7 mg) and sodium bicarbonate (128 mg) in methanol was stirred at room temperature under argon for 6 h. A spatula tip of sodium bicarbonate was then added, and the mixture was stirred overnight. It was diluted with ethyl acetate, water was added, and separation of the phases was followed by extraction with ethyl acetate. Washing of the combined organic phases with brine, drying over sodium sulphate, concentration and column chromatography on silica gel resulted in the title compound as a viscous oil (29 mg).

1H-NMR (ppm, CDCl₃, 400 MHz): 1.63 (s, 3H, Me), 1.69 (s, 3H, Me), 2.56 (s, 3H, Me), 2.94-3.01 (m, 3H), 5.48 (br. s, 1H, OH), 6.74-6.77 (m, 2H), 6.84-6.93 (m, 2H), 7.21-7.25 (m, 2H), 7.43 (dd, J=9.0, 6.1 Hz, 1H), 7.57-7.59 (dd, J=8.6, 2.3 Hz, 1H), 8.22 -8.23 (m, 1H), 8.31 (d, J=8.6 Hz, 1H), 8.80 (br. s, 1H, NH). C₂₉H₂₄CIFN₂Oₛ (534.98): LC-MS: m/z 535 [M+H⁺].

### rac-6-{2-[2-(2-Chloro-4-fluorophenyl)-2-methylpropyl]-2-hydroxydec-3-ynoylamino}-4-methyl-2,3-benzoxazin-1-one 8

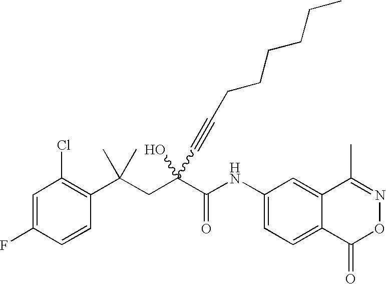

Reaction of 1-octyne (0.4 ml), nBuLi (0.6 ml, 1.6 M in hexane) and 6-[4-(2-chloro-4-fluorophenyl)-4-methyl-2-oxovaleroylamino]-4-methyl-2,3-benzoxazin-1-one (110 mg) in THF (3 ml) at −78° C. as described for Example 1 gave, after column chromatography on silica gel, a white solid (25 mg).

1H-NMR (ppm, CDCl₃, 400 MHz): 0.87 (t, J=7.0 Hz, 3H), 1.26-1.46 (m, 8H), 1.58 (s, 3H, Me), 1.63 (s, 3H, Me), 2.12 (t, J=7.0 Hz, CH₂C≡C), 2.56 (s, 3H, Me) 2.79-2.91 (m, 3H), 6.92-6.95 (m, 2H), 7.40 (dd, J=8.9, 6.3 Hz, 1H), 7.53 (dd, J=8.6, 1.9 Hz, 1H), 8.21 (d, J=1.9 Hz, 1H), 8.30 (d, J=8.6 Hz, 1H), 8.71 (br. s., 1H, NH); C₂₉H₃₂ClFN₂O₄ (527.0): LC-MS: m/z=527 [M+H⁺].

### rac-6-{2-[2-(2-Chloro-4-fluorophenyl)-2-methyl propyl]-2-hydroxy-5-phenylpent-3-ynoylamino}-4-methyl-2,3-benzoxazin-1-one 9

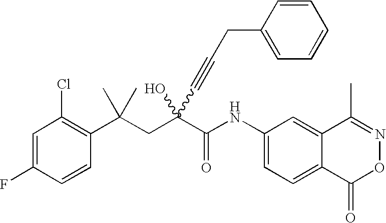

Reaction of 3-phenyl-1-propyne (0.17 ml), nBuLi (0.51 ml, 1.6 M in hexane) and 6-[4-(2-chloro-4-fluorophenyl)-4-methyl-2-oxovaleroylamino]-4-methyl-2,3-benzoxazin-1-one (140 mg) in THF (3 ml) at −78° C. as described for Example 1 gave, after column chromatography on silica gel and drying in vacuo, a white foam (116 mg).

1H-NMR (ppm, CDCl₃, 400 MHz): 1.59 (s, 3H, Me), 1.61 (s, 3H, Me), 2.55 (s, 3H, Me), 2.79-2.95 (m, 3H), 3.4-3.6 (m, 2H, CH₂C≡C), 6.8-6.93 (m, 2H), 7.23-7.42 (m, 7H), 8.15 (d, J=2.3 Hz, 1H), 8.27 (d, J=8.6 Hz, 1H), 8.64 (br. s., 1H, NH). C₃₀H₂₆ClFN₂O₄ (533.0): LC-MS: m/z=533 [M+H⁺].

### rac-6-{4-(2-Chloro-4-fluorophenyl)-2-ethynyl-2-hydroxy-4-methylpentanoylamino}-4-methyl-2,3-benzoxazin-1-one 10

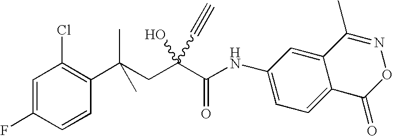

Ethynylmagnesium bromide (2.2 ml, 0.5 M in THF) was added to an ice-cold solution of 6-[4-(2-chloro-4-fluorophenyl)-4-methyl-2-oxovaleroylamino]-4-methyl-2,3-benzoxazin-1-one (208 mg) in THF (4 ml). Under argon, the reaction solution was allowed to reach room temperature over the course of 3 h. Working up as described in Example 1 and column chromatography on silica gel resulted in the title compound as a foam (84 mg) after oil-pump drying. ¹H-NMR (ppm, CDCl₃, 400 MHz): 0.8-0.9 (m, 1H), 1-58 (s, 3H, Me), 1.65 (s, 3H, Me), 2.56-2.96 (6H), 6.86-6.94 (m, 2H), 7.41 (dd, J=9.0, 6.2 Hz, 1H), 7.56 (dd, J=8.6, 1.9 Hz, 1H), 8.19 (d, J=1.9 Hz, 1H), 8.31 (d, J=8.6 Hz, 1H), 8.63 (br. s., 1H, NH).

C₂₃H₂₀ClFN₂O₄ (542.9): LC-MS: m/z=543 [M+H⁺].

### rac-6-{4-(2-Chloro-4-fluorophenyl)-2-hydroxy-4-methyl-2-vinyl-pentanoylamino}-4-methyl-2,3-benzoxazin-1-one 11

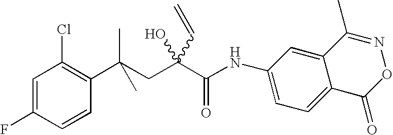

A vinylmagnesium bromide solution (0.5 ml, 1M in THF) was injected into 6-[4-(2-chloro -4-fluorophenyl)-4-methyl-2-oxovaleroylamino]-4-methyl-2,3-benzoxazin-1-one (103 mg) in THF (3 ml) at −78° C., and the mixture was allowed to reach room temp. under argon overnight. Addition of aqueous ammonium chloride solution was followed by extraction with ethyl acetate and washing with sat. sodium chloride solution. Drying with sodium sulphate was followed by concentration in a rotary evaporator and purification by column chromatography on silica gel to result in the title compound as solidified oil (18 mg). 1H-NMR (ppm, CDCl₃, 400 MHz, selected signals): 1.53 (s, 3H, Me), 1.57 (s, 3H, Me), 2.35 (s, 1H), 2.56 (s, 3H, Me), 2.74 (d, J=15.3 Hz, 1H), 2.89 (d, J=15.3 Hz, 1H), 5.15 (d, J=10.5 Hz, 1H), 5.27 (d, J=17.6 Hz, 1H), 6.10 (dd, J=17.2, 10.6 Hz, 1H), 6.81-6.86 (m, 1H),

### rac-6-{4-(2-Chloro-4-fluorophenyl)-2-hydroxy-2-[(4-methoxyphenyl)ethynyl]-4-methylpentanoylamino}-4-methyl-2,3-benzoxazin-1-one 12

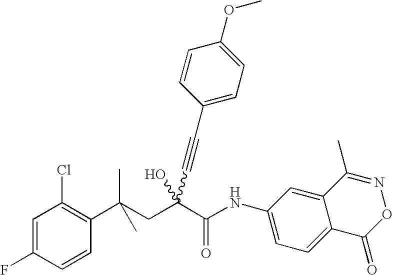

Reaction of 4-methoxyphenylacetylene (0.4 ml), nBuLi (0.6 ml, 1.6 M in hexane) and 6-[4-(2-chloro-4-fluorophenyl)-4-methyl-2-oxovaleroylamino]-4-methyl-2,3-benzoxazin-1-one (110 mg) at −78° C. as described for Example 1 gave, after column chromatography on silica gel, the title compound as a white solid (44 mg).

1H-NMR (ppm, CDCl₃, 400 MHz): 1.63 (s, 3H, Me), 1.69 (s, 3H, Me), 2.91-3.01. (m, 3H), 3.81 (s, 3H, Me), 6.81-6.94 (m, 3H), 7.25-7.29 (m, 3H), 7.43 (dd, J=8.4, 6.3 Hz, 1H), 7.58 (dd, J=8.6, 2.3 Hz, 1H), 8.24 (d, J=1.9 Hz, 1H), 8.31 (d, J=8.6 Hz, 1H), 8.79 (br. s., 1H, NH). C₃₀H₂₆ClFN₂O₅ (549.0): LC-MS: m/z=549 [M+H⁺].

### rac-6-{4-(2-Chloro-4-fluorophenyl)-2-hydroxy-4-methyl-2-(phenylethynyl)pentanoylamino}-4-methyl-2,3-benzoxazin-1-one 13

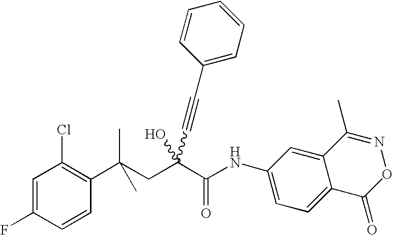

Lithium phenylacetylide (0.65 ml, 1M in THF) was added to 6-[4-(2-chloro-4-fluorophenyl)-4-methyl-2-oxovaleroylamino]-4-methyl-2,3-benzoxazin-1-one (136 mg) at. −78° C. and the mixture was stirred at −78° C. under argon for 2.5 h. Working up as described for Example 1 and column chromatography on silica gel resulted in the title compound as a white foam (102 mg) after oil-pump drying.

1H-NMR (ppm, CDCl₃, 400 MHz): 1.64 (s, 3H, Me), 1.70 (s, 3H, Me), 2.57 (s, 3H, Me), 2.92-3.03 (m, 3H), 6.82-6.86 (m, 1H), 6.91-6.93 (m, 1H), 7.30-7.36 (m, 5H), 7.44 (dd, J=9.0, 6.2 Hz, 1H), 7.59 (dd, J=8.6, 2.0 Hz, 1H), 8.23 (d, J=2.0 Hz, 1H), 8.31 (d, J=8.2 Hz, 1H), 8.79 (br. s., NH); C₂₉H₂₄ClFN₂O₄ (519.0): HPLC-MS: m/z=518 [M].

The compounds 14 and 15 were prepared in analogy to Example 10 from 6-(4-methyl -4-phenyl-2-oxovaleroylamino)-4-methyl-2,3-benzoxazin-1-one and the alkynyl-magnesium halide:

### rac-6-[2-Ethynyl-2-hydroxy-4-methyl-4-phenylpentanoylamino]-4-methyl-2,3-benzoxazin-1-one 14

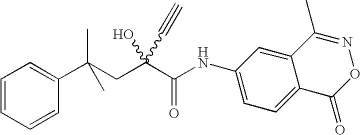

1H-NMR (ppm, CDCl₃, 400 MHz): 1.42 (3H), 1.59 (3H), 2.57 (3H), 2.64 (4H), 7.15 (1H), 7.31 (2H), 7.46 (2H), 7.58 (1H), 8.25 (1H), 8.30 (1H), 8.81 (1H).

### rac-6-[2-Hydroxy-4-methyl-4-phenyl-2-propynylpentanoylamino]-4-methyl-2,3-benzoxazin-1-one 15

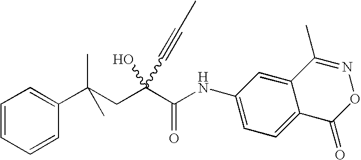

1H-NMR (ppm, CDCl₃, 400 MHz): 1.42 (3H), 1.59 (3H), 2.50-2.65 (6H), 7.11 (1H), 7.30 (2H), 7.43 (2H), 7.58 (1H), 8.29 (2H), 8.85 (1H).

The compounds 15-28 were prepared in analogy to Example 1 from 6-(4-methyl-4-phenyl-2-oxovaleroylamino)-4-methyl-2,3-benzoxazin-1-one and the respective lithium arylacetylide:

### rac-6-[2-Hydroxy-4-methyl-4-phenyl-2-(phenylethynyl)pentanoylamino]-4-methyl -2,3-benzoxazin-1-one 16

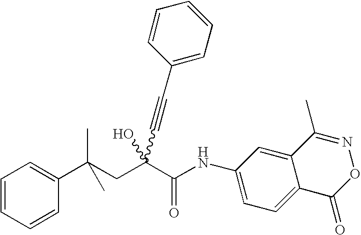

1H-NMR (ppm, CDCl₃, 300 MHz): 1.47 (3H), 1.65 (3H), 2.57 (3H), 2.62-2.78 (3H), 7.15 (1H), 7.27-7.37 (5H), 7.40 (2H), 7.50 (2H), 7.59 (1H), 8.29 (2H), 8.90 (1H).

### (+)-6-[2-Hydroxy-4-methyl-2-[(4-methyl phenyl)ethynyl]-4-phenyl pentanoylamino]-4-methyl-2,3-benzoxazin-1-one 17a and (−)-6-[2-Hydroxy-4-methyl-2-[(4-methylphenyl)ethynyl]-4-phenylpentanoylamino]-4-methyl-2,3-benzoxazin-1-one 17b

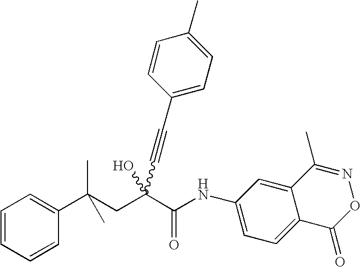

1H-NMR (ppm, CDCl₃, 300 MHz): 1.48 (3H), 1.64 (3H), 2.36 (3H), 2.57 (3H), 2.60-2.80 (3H), 7.08-7.20 (3H), 7.30 (4H), 7.49 (2H), 7.60 (1H), 8.29 (2H), 8.90 (1H).

16a: [α]^{D}₂₀: +28.4° (CHCl₃, 1.03 g/100 ml; λ=589 nm)

16b: [α]^{D}₂₀: −28.6° (CHCl₃, 1.01 g/100 ml; λ=589 nm)

### rac-6-[2-Hydroxy-2-[(4-methoxyphenyl)ethynyl]-4-methyl-4-phenylpentanoylamino]-4-methyl-2,3-benzoxazin-1-one 18

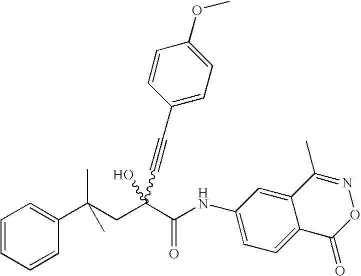

1H-NMR (ppm, CDCl₃, 300 MHz): 1.47 (3H), 1.63 (3H), 2.56 (3H), 2.60-2.78 (3H), 3.80 (3H), 6.81 (2H), 7.13 (1H), 7.25-7.38 (4H), 7.48 (2H), 7.60 (1H), 8.28 (2H), 8.89 (1H).

### (+)-6-[2-Hydroxy-2-[(4-methoxyphenyl)ethynyl]-4-methyl-4-phenylpentanoylamino]-4-methyl-2,3-benzoxazin-1-one 18a and (−)-6-[2-Hydroxy-2-[(4-methoxyphenyl)ethynyl]-4-methyl-4-phenylpentanoylamino]-4-methyl-2,3-benzoxazin-1-one 18b

The racemic mixture which was described in example 18 was separated by preparative chiral HPLC (column Chiralpak AD 250×10 mm) into the enantiomers 18a and 18b.

18a: [α]^{D}₂₀: +29.3° (CHCl₃, 1.12 g/100 ml; λ=589 nM)

18b: ([α]^{D}₂₀: −30.0° (CHCl₃, 1.14 g/100 ml; λ=589 nM)

### rac-6-[2-Hydroxy-2-[(4-(N,N-dimethylamino)phenyl)ethynyl]4-methyl-4-phenylpentanoylamino]-4-methyl-2,3-benzoxazin-1-one 19

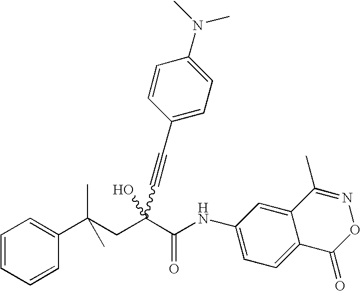

1H-NMR (ppm, CDCl₃, 400 MHz): 1.48 (3H), 1.62 (3H), 2.57 (3H), 2.60-2.75 (3H), 2.98 (6H), 6.58 (2H), 7.12 (1H), 7.23-7.38 (4H), 7.48 (2H); 7.57 (1H), 8.28 (2H), 8.90 (1H).

### rac-6-[2-Hydroxy-4-methyl-4-phenyl-2-[(4-trifluoromethyl phenyl)ethynyl]-pentanoylamino]-4-methyl-2,3-benzoxazin-1-one 20

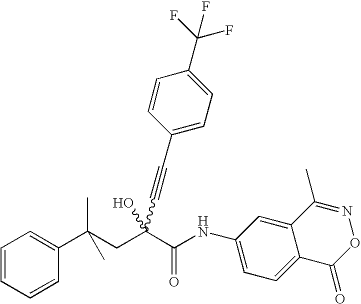

1H-NMR (ppm, CDCl₃, 400 MHz): 1.48 (3H), 1.63 (3H), 2.57 (3H), 2.64-2.80 (3H), 7.17 (1H), 7.33 (2H), 7.48 (4H), 7.56 (2H), 7.61 (1H), 8.30 (2H), 8.92 (1H).

### (+)-6-[2-Hydroxy-4-methyl-4-phenyl-2-[(4-trifluormethylphenyl)ethynyl]-pentanoylamino]-4-methyl-2,3-benzoxazin-1-one 20a and (−)-6-[2-Hydroxy-4-methyl-4-phenyl-2-[(4-trifluormethylphenyl)ethynyl]-pentanoylamino]-4-methyl-2,3-benzoxazin-1-one 20b

The racemic mixture which was described in example 20 was separated by preparative chiral HPLC (column Chiralpak AD 250×10 mm) into the enantiomers 20a and 20b.

20a: [α]^{D}₂₀: +19.9° (CHCl₃, 1.05 g/100 ml; λ=589 nM)

20b: [α]^{D}₂₀: −20.4° (CHCl₃, 1.01 g/100 ml; λ=589 nM)

### rac-6-[2-[(4-Cyanophenyl)ethynyl]-2-hydroxy-4-methyl-4-phenyl pentanoylamino]-4-methyl-2,3-benzoxazin-1-one 21

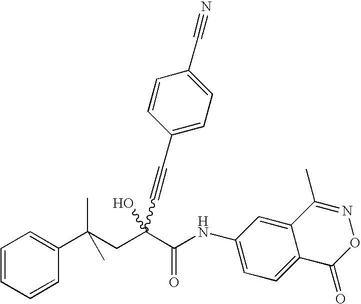

1H-NMR (ppm, CDCl₃, 400 MHz): 1.50 (3H), 1.62 (3H), 2.57 (3H), 2.63-2.82 (3H), 7.18 (1H), 7.35 (2H), 7.48 (4H), 7.55-7.68 (2H), 7.62 (1H), 8.30 (2H), 8.94 (1H).

### (+)-6-[2-[(4-Cyanophenyl)ethynyl]-2-hydroxy-4-methyl-4-phenylpentanoylamino]-4-methyl-2,3-benzoxazin-1-one 21a and (−)-6-[2-[(4-Cyanophenyl)ethynyl]-2-hydroxy-4-methyl-4-phenylpentanoylamino]-4-methyl-2,3-benzoxazin-1-one 21b

The racemic mixture which was described in example 21 was separated by preparative chiral HPLC (column Chiralpak AD 250×10 mm) into the enantiomers 21a and 21b.

21a: [α]D₂₀: +26.6° (CHCl₃, 1.12 g/100 ml; λ=589 nM)

21b: [α]^{D}₂₀: −26.8° (CHCl₃, 1.02 g/100 ml; λ=589 nM)

### rac-6-[2-Hydroxy-4-methyl-4-phenyl-2-[(4-phenylphenyl)ethynyl]pentanoylamino]-4-methyl-2,3-benzoxazin-1-one 22

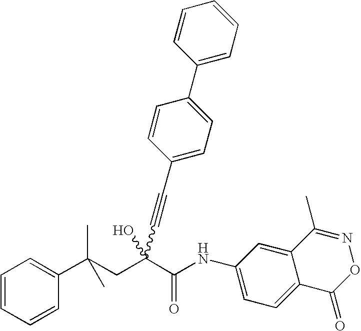

1H-NMR (ppm, CDCl₃, 400 MHz): 1.50 (3H), 1.68 (3H), 2.58 (3H), 2.64-2.81 (3H), 7.18 (1H), 7.30-7.40 (3H), 7.41-7.61 (11H), 8.30 (2H), 8.92 (1H).

### (+)-6-[2-Hydroxy-4-methyl-4-phenyl-2-[(4-phenyl phenyl)ethynyl]pentanoylamino]-4-methyl-2,3-benzoxazin-1-one 22a and (−)-6-[2-Hydroxy-4-methyl-4-phenyl-2-[(4-phenylphenyl)ethynyl]pentanoylamino]-4-methyl-2,3-benzoxazin-1-one 22b

The racemic mixture which was described in example 22 was separated by preparative chiral HPLC (column Chiralpak AD 250×10 mm) into the enantiomers 22a and 22b.

22a: [α]^{D}₂₀: +38.4° (CHCl₃, 1.06 g/100 ml; λ=589 nM)

22b: [α]^{D}₂₀: −30.60 (CHCl₃, 1.12 g/100 ml; λ=589 nM)

### rac-6-[2-Hydroxy-4-methyl-4-phenyl-2-[(3-trifluoromethylphenyl)ethynyl]-pentanoylamino]-4-methyl-2,3-benzoxazin-1-one 23

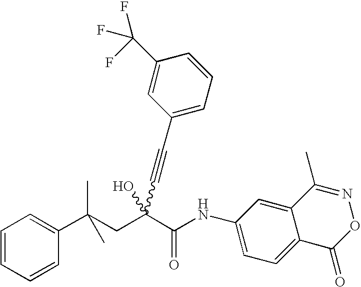

1H-NMR (ppm, CDCl₃, 300 MHz): 1.52 (3H), 1.68 (3H), 2.60 (3H), 2.65-2.88 (3H), 7.21 (1H), 7.49 (2H), 7.42-7.70 (7H), 8.34 (2H), 8.96 (1H).

### rac-6-[2-Hydroxy-4-methyl-4-phenyl-2-[(2-trifluoromethylphenyl)ethynyl]-pentanoylamino]-4-methyl-2,3-benzoxazin-1-one 24

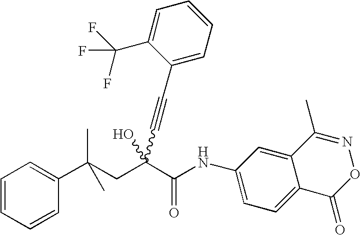

1H-NMR (ppm, CDCl₃, 600 MHz): 1.52 (3H), 1.65 (3H), 2.62 (3H), 2.69 (1H), 2.78 (1H), 2.91 (1H), 7.11 (1H), 7.32 (3H), 7.51 (3H), 7.57 (2H), 7.70 (1H), 8.20 (1H), 8.45 (1H), 8.75 (1H).

### (+)-6-[2-Hydroxy-4-methyl-4-phenyl-2-[(2-trifluormethylphenyl)ethynyl]-pentanoylamino]-4-methyl-2,3-benzoxazin-1-one 24a and (−)-6-[2-Hydroxy-4-methyl-4-phenyl-2-[(2-trifluormethylphenyl)ethynyl]-pentanoylamino]-4-methyl-2,3-benzoxazin-1-one 24b

The racemic mixture which was described in example 24 was separated by preparative chiral HPLC (column Chiralpak AD 250×10 mm) into the enantiomers 24a and 24b.

24a: [α]^{D}₂₀: +21.3° (CHCl₃, 1.00 g/100 ml; λ=589 nM)

24b: [α]^{D}₂₀: 19.4° (CHCl₃, 1.00 g/100 ml; λ=589 nM)

### rac-6-[2-Hydroxy-4-methyl-2-[(4-nitrophenyl)ethynyl]-4-phenylpentanoylamino]-4-methyl-2,3-benzoxazin-1-one 25

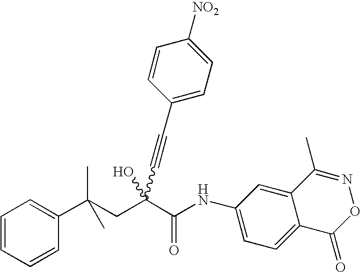

1H-NMR (ppm, CDCl₃, 600 MHz): 1.47 (3H), 1.62 (3H), 2.55 (3H), 2.79 (1H), 2.81 (2H), 7.18 (1H), 7.34 (2H), 7.50 (4H), 7.63 (1H), 8.17 (2H), 8.80 (2H), 8.94 (1H).

### rac-6-[2-[[4-(1,1-Dimethylethyl)phenyl]ethynyl]-2-hydroxy-4-methyl -4-phenylpentanoylamino]-4-methyl-2,3-benzoxazin-1-one 26

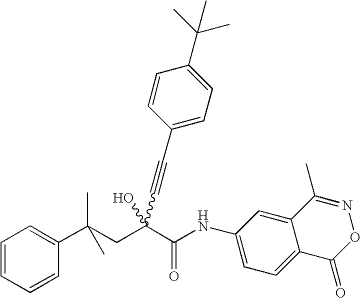

1H-NMR (ppm, CDCl₃, 300 MHz): 1.32 (9H), 1.51 (3H), 1.68 (3H), 2.62 (3H), 2.65-2.82 (3H), 7.18 (1H), 7.30-7.40 (6H), 7.52 (2H), 7.63 (1H), 8.32 (2H), 8.93 (1H).

### rac-6-[2-Hydroxy-4-methyl-2-[(3-methylphenyl)ethynyl]-4-phenyl pentanoylamino]-4-methyl-2,3-benzoxazin-1-one 27

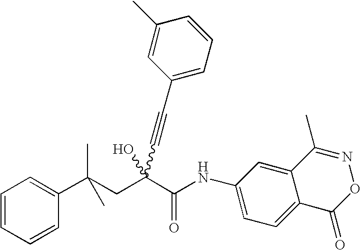

1H-NMR (ppm, CDCl₃, 400 MHz): 1.47 (3H), 1.63 (3H), 2.30 (3H), 2.58 (3H), 2.62-2.80 (3H), 7.12-7.26 (5H), 7.32 (2H), 7.50 (2H), 7.60 (1H), 8.30 (2H), 8.90 (1H).

### rac-6-[2-Hydroxy-4-methyl-2-[(2-methylphenyl)ethynyl]-4-phenylpentanoylamino]-4-methyl-2,3-benzoxazin-1-one 28

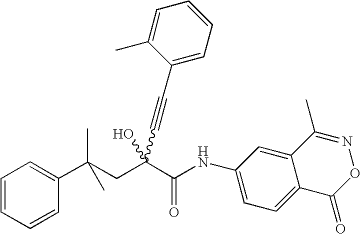

1H-NMR (ppm, CDCl₃, 300 MHz): 1.47 (3H), 1.65 (3H), 2.38 (3H), 2.58 (3H), 2.62-2.80 (3H), 7.08-7.42 (7H), 7.49 (2H), 7.60 (1H), 8.22-8.36 (2H), 8.90 (1H).

### rac-6-[2-(3,3-Dimethylbutynyl)-2-hydroxy-4-methyl-4-phenyl pentanoylamino]-4-methyl-2,3-benzoxazin-1-one 29

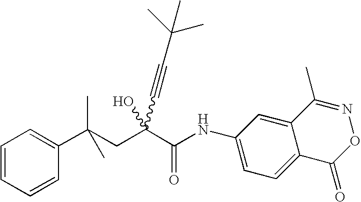

1H-NMR (ppm, CDCl₃; 300 MHz): 1.20 (9H), 1.43 (3H), 1.60 (3H), 2.46 (1H), 2.50-2.63 (5H), 7.11 (1H), 7.28 (2H), 7.43 (2H), 7.54 (1H), 8.22 (1H), 8.29 (1H), 8.32 (1H).

The following compound was prepared in analogy to Example 7 from the compound described in Example 13 and 4′-iodoacetophenone:

### rac-6-[2-[(4-Acetylphenyl)ethynyl]-2-hydroxy-4-methyl-4-phenylpentanoylamino]-4-methyl-2,3-benzoxazin-1-one 30

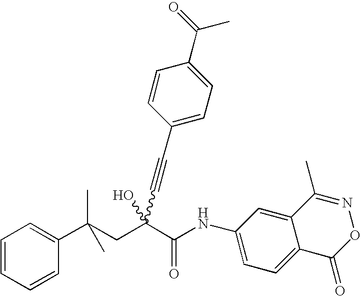

1H-NMR (ppm, CDCl₃, 300 MHz): 1.48 (3H), 1.63 (3H), 2.56 (3H), 2.60 (3H), 2.63-2.82 (3H), 7.18 (1H), 7.33 (2H), 7.40-7.56 (4H), 7.62 (1H), 7.90 (2H), 8.30 (2H), 8.93 (1H).

### (+)-6-[2-[(4-Acetylphenyl)ethynyl]-2-hydroxy-4-methyl-4-phenylpentanoylamino]-4-methyl-2,3-benzoxazin-1-one 30a and (−)-6-[2-[(4-Acetylphenyl)ethynyl]-2-hydroxy-4-methyl-4-phenylpentanoylamino]-4-methyl-2,3-benzoxazin-1-one 30b

The racemic mixture which was described in example 30 was separated by preparative chiral HPLC (column Chiralpak AD 250×10 mm) into the enantiomers 30a and 30b.

30a: [α]^{D}₂₀: +31.3° (CHCl₃, 1.09 g/100 ml; λ=589 nM)

30b: [α]^{D}₂₀: −28.4° (CHCl₃, 1.09 g/100 ml; λ=589 nM)

The compounds 30 and 31 were prepared in analogy to Example 1 from 6-[3-[1-(2-fluoro-5-trifluoromethylphenyl)cyclopropyl]-2-oxopropionylamino]-4-methyl-2,3-benzoxazin-1-one

### rac-6-[2-[(2-Fluoro-5-trifluoromethylphenyl)cyclopropylmethyl]-2-hydroxy-4-(4-trifluoromethylphenyl)but-3-inoylamino]-4-methyl-2,3-benzoxazin-1-one 31

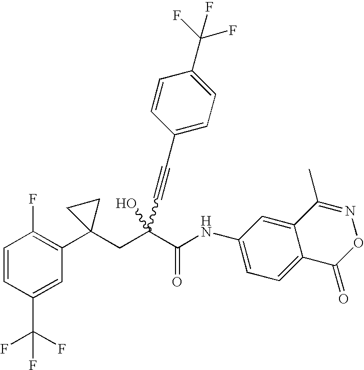

1H-NMR (ppm, CDCl₃, 400 MHz): 0.90 (1H), 1.00-1.15 (3H), 2.51 (1H), 2.55 (3H), 2.68 (1H), 3.18 (1H), 7.01 (1H), 7.30 (1H), 7.41 (2H), 7.56 (2H), 7.63 (1H), 7.68 (1H), 8.19 (1H), 8.31 (1H), 8.98 (1H).

### (+)-6-[2-[(2-Fluor-5-trifluormethylphenyl)cyclopropylmethyl]-2-hydroxy-4-(4-trifluormethylphenyl)but-3-inoylamino]-4-methyl-2,3-benzoxazin-1-one 31a and (−)-6-[2-[(2-Fluor-5-trifluormethylphenyl)cyclopropylmethyl]-2-hydroxy-4-(4-trifluormethylphenyl)but-3-inoylamino]-4-methyl-2,3-benzoxazin−1-one 31b

The racemic mixture which was described in, example 31 was separated by preparative chiral HPLC (column Chiralpak AD 250×10 mm) into the enantiomers 31a and 31b.

31a: [α]^{D}₂₀: +2.3° (CHCl₃, 1.00 g/100 ml; λ=589 nM)

31b: [α]^{D}₂₀: −1.9° (CHCl₃, 1.00 g/100 ml; λ=589 nM)

### rac-6-[2-[(2-Fluoro-5-trifluoromethylphenyl)cyclopropylmethyl]-2-hydroxy-4-(4-methylphenyl)but-3-ynoylamino]-4-methyl-2,3-benzoxazin-1-one 32

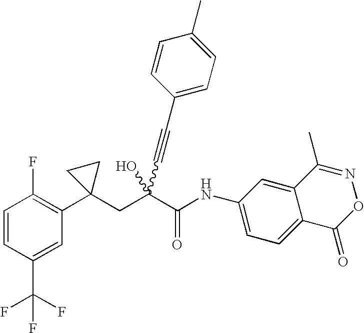

1H-NMR (ppm, CDCl₃, 400 MHz): 0.88 (1H), 0.98-1.13 (3H), 2.34 (3H), 2.44 (1H), 2.55 (3H), 2.70 (1H), 3.02 (1H), 7.01 (1H), 7.10 (2H), 7.22 (2H), 7.30 (1H), 7.64 (2H), 8.19 (1H), 8.31 (1H), 8.98 (1H).

### (+)-6-[2-[(2-Fluor-5-trifluormethylphenyl)cyclopropylmethyl]-2-hydroxy-4-(4-methylphenyl)but-3-inoylamino]-4-methyl-2,3-benzoxazin-1-one 32a and (−)-6-[2-[(2-Fluor-5-trifluormethylphenyl)cyclopropylmethyl]-2-hydroxy-4-(4-methylphenyl)but-3-inoylamino]-4-methyl-2,3-benzoxazin−1-one 32b

The racemic mixture which was described in example 32 was separated by preparative chiral HPLC (column Chiralpak AD 250×10 mm) into the enantiomers 32a and 32b.

32a: [α]^{D}₂₀: +8.6° (CHCl₃, 1.00 g/100 ml; λ=589 nM)

32b: [α]D₂₀: −8.7° (CHCl₃, 1.00 g/100 ml; λ=589 nM)

The following compound was prepared in analogy to example 7 from compound which was described in example 14 and 3′-Iodacetophenon:

### rac-6-[2-[(3-Acetylphenyl)ethynyl]-2-hydroxy-4-methyl-4-phenylpentanoylamino]-4-methyl-2,3-benzoxazin-1-one 33

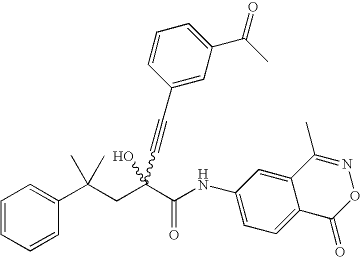

1H-NMR (ppm, CDCl₃, 300 MHz): 1.49 (3H), 1.63 (3H), 2.57 (6H), 2.62-2.81 (3H), 7.16 81H), 7.28-7.70 (7H), 7.90-8.00 (2H), 8.30 (2H), 8.94 (1H).

Compounds 34 and 35 were prepared in analogy to example 1 from 6-(4-Methyl-4-phenyl-2-oxovaleroylamino)-4-methyl-2,3-benzoxazin-1-one and the according Lithium arylacetylide.

### rac-6-[2-[(2,5-Dimethylphenyl)ethynyl]-2-hydroxy-4-methyl-4-phenylpentanoylamino]-4-methyl-2,3-benzoxazin-1-one 34

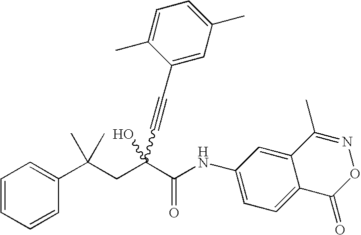

1H-NMR (ppm, CDCl₃, 400 MHz): 1.49 (3H), 1.62 (3H), 2.27 (3H), 2.33 (3H), 2.57 (3H), 2.65-2.78 (3H), 7.03 (2H), 7.13 (2H), 7.30 (2H), 7.50 (2H), 7.61 (1H), 8.22 (1H), 8.30 (1H), 8.89 (1H).

### rac-6-[2-[(2,4,5-Trimethylphenyl)ethynyl]-2-hydroxy-4-methyl-4-phenylpentanoylamino]-4-methyl-2,3-benzoxazin-1-one 35

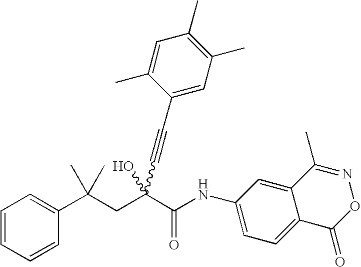

1H-NMR (ppm, CDCl₃, 400 MHz): 1.47 (3H), 1.64 (3H), 2.18 (3H), 2.21 (3H), 2.30 (3H), 2.56 (3H), 2.65-2.77 (3H), 6.93 (1H), 7.12 (2H), 7.30 (2H), 7.48 (2H), 7.59 (1H), 8.22 (1H), 8.29 (1H), 8.90 (1H).

### (+)-6-[2-[(2,4,5-Trimethylphenyl)ethynyl]-2-hydroxy-4-methyl-4-phenylpentanoylamino]-4-methyl-2,3-benzoxazin-1-one 35a and (−)-6-[2-[(2,4,5-Trimethylphenyl)ethynyl]-2-hydroxy-4-methyl-4-phenylpentanoylamino]-4-methyl-2,3-benzoxazin-1-one 35b

The racemic mixture which was described in example 35 was separated by preparative chiral HPLC (column Chiralpak AD 250×10 mm) into the enantiomers 35a and 35b.

35a: [α]^{D}₂₀: +30.6° (CHCl₃, 0.97 g/100 ml; λ=589 nM)

35b: [α]^{D}₂₀: −28.0° (CHCl₃, 0.96 g/100 ml; λ=589 nM)

The following compound was prepared in analogy to example 9 from 3-Phenyl-1-propine, nBuLi and 6-(4-Methyl-4-phenyl-2-oxovaleroylamino)-4-methyl-2,3-benzoxazin -1-one:

### rac-6-{2-(2-phenyl)-2-methylpropyl]-2-hydroxy-5-phenylpent-3-inoylamino}-4-methyl-2,3-benzoxazin-1-one 36

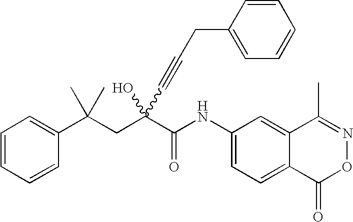

1H-NMR (ppm, CDCl₃, 400 MHz): 1.42 (3H), 1.53 (3H), 2.55-2.70 (6H), 3.58 (2H), 7.11 (1H), 7.20-7.35 (7H), 7.41 (2H), 7.48 (1H), 8.20 (1H), 8.28 (1H), 8.80 (1H).

Compounds 37 and 38 were prepared in analogy to example 1 from 6-(4-Methyl-4-(2-chlor-6-fluorphenyl)-2-oxovaleroylamino)-4-methyl-2,3-benzoxazin-1-one and the according Lithium arylacetylide.

### rac-6-[2-Hydroxy-4-methyl-4-(2-chlor-6-fluorphenyl)-2-(4-methylphenylethinyl)pentanoylamino]-4-methyl-2,3-benzoxazin-1-one 37

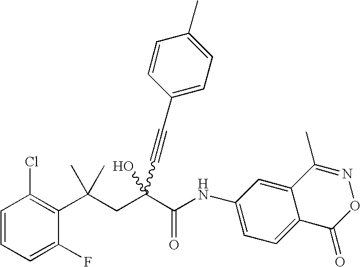

1H-NMR (ppm, CDCl₃, 300 MHz): 1.73 (3H), 1.82 (3H), 2.33 (3H), 2.57 (3H), 2.88-3.02 (3H), 6.75-6.96 (2H), 7.01 (1H), 7.09 (2H), 7.27 (2H), 7.60 (1H), 8.22-8.35 (2H), 8.96 (1H).

### (+)-6-[2-Hydroxy-4-methyl-4-(2-chlor-6-fluorphenyl)-2-(4-methylphenylethynyl)pentanoylamino]-4-methyl-2,3-benzoxazin-1-one 37a and (−)-6-[2-Hydroxy-4-methyl-4-(2-chlor-6-fluorphenyl)-2-(4-methylphenylethynyl)pentanoylamino]-4-methyl-2,3-benzoxazin-1-one 37b

The racemic mixture which was described in example 37 was separated by preparative chiral HPLC (column Chiralpak AD 250×10 mm) into the enantiomers 37a and 37b.

37a: [α]^{D}₂₀: +21.5° (CHCl₃, 100 g/100 ml; λ=589 nM)

37b: [α]^{D}₂₀: −21.0° (CHCl₃, 1.04 g/100 ml; λ=589 nM)

### rac-6-[2-Hydroxy-4-methyl-4-(2-chlor-6-fluorphenyl)-2-(4-(trifluormethyl)phenylethynyl)pentanoylamino]-4-methyl-2,3-benzoxazin-1-one 38

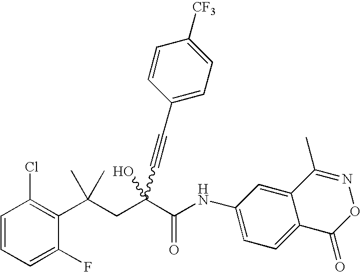

1H-NMR (ppm, CDCl₃, 400 MHz): 1.60 (3H), 1.93 (3H), 2.36 (1H), 2.56-2.72 (5H), 7.04 (1H), 7.14 (2H), 7.45 (2H), 7.53 (2H), 7.80 (1H), 8.35-8.45 (2H), 8.90 (1H).

### (+)-6-[2-Hydroxy-4-methyl-4-(2-chlor-6-fluorphenyl)-2-(4-(trifluormethyl)phenylethynyl)pentanoylamino]-4-methyl-2,3-benzoxazin-1-one 38a and (−)-6-[2-Hydroxy-4-methyl-4-(2-chlor-6-fluorphenyl)-2-(4-(trifluormethyl)phenylethynyl)pentanoylamino]-4-methyl-2,3-benzoxazin-1-one 38b

The racemic mixture which was described in example 38 was separated by preparative chiral HPLC (column Chiralpak AD 250×10 mm) into the enantiomers-38a and 38b.

38a: [α]^{D}₂₀: +143.2° (CHCl₃, 1.05 g/100 ml; λ=589 nM)

38b: [α]20: 137.8° (CHCl₃, 1.12 g/100 ml; λ=589 nM)

Compounds 39 and 40 were prepared in analogy to example 1 from 6-(4-Methyl-4-(2-chlorphenyl)-2-oxovaleroylamino)-4-methyl-2,3-benzoxazin-1-one and the according Lithium arylacetylide.

### rac-6-[2-(2-Chlorphenyl)cyclopropylmethyl]-2-hydroxy-4-(4-methylphenyl)but-3-inoylamino]-4-methyl-2,3-benzoxazin-1-one 39

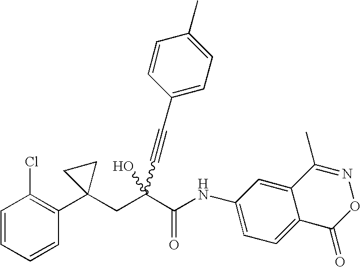

1H-NMR (ppm, CDCl₃, 400 MHz): 0.84 (1H), 1.00 (1H), 1.08-1.22 (2H), 2.36 (3H), 2.53 (3H), 2.90 (1H), 7.03-7.18 (4H), 7.23-7.38 (3H), 7.50 (1H), 7.60 (1H), 8.22 (1H), 8.29 (1H), 8.91 (1H).

### (+)-6-[2-(2-Chlorphenyl)cyclopropylmethyl]-2-hydroxy-4-(4-methylphenyl)but-3-inoylamino]-4-methyl-2,3-benzoxazin-1-one 39a and (−)-6-[2-(2-Chlorphenyl)cyclopropylmethyl]-2-hydroxy-4-(4-methylphenyl)but-3-inoylamino]-4-methyl-2,3-benzoxazin-1-one 39b

The racemic mixture which was described in example 39 was separated by preparative chiral HPLC (column Chiralpak AD 250×10 mm) into the enantiomers 39a and 39b.

39a: [α]^{D}₂₀: +30.8° (CHCl₃, 1.00 g/100 ml; λ=589 nM)

39b: [α]^{D}₂₀: −28.3° (CHCl₃, 1.00 g/100 ml; λ=589 nM)

### rac-6-[2-(2-Chlorphenyl)cyclopropylmethyl]-2-hydroxy-4-(4-trifluormethylphenyl)but -3-inoylamino]-4-methyl-2,3-benzoxazin−1-one 40

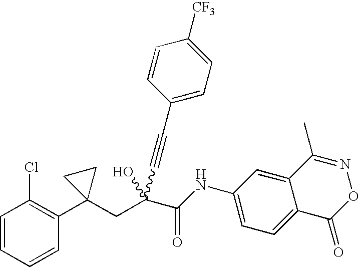

1H-NMR (ppm, CDCl₃, 300 MHz): 0.91 (1H), 1.02 (1H), 1.08-1.25 (2H), 2.53 (3H), 3.00 (1H), 7.02-7.18 (2H), 7.28 (1H), 7.42-7.54 (3H), 7.55-7.67 (3H), 8.22 (1H), 8.32 (1H), 8.91 (1H).

### (+)-6-[2-(2-Chlorphenyl)cyclopropylmethyl]-2-hydroxy-4-(4-trifluormethylphenyl)but -3-inoylamino]-4-methyl-2,3-benzoxazin-1-one 40a and (−)-6-[2-(2-Chlorphenyl)cyclopropylmethyl]-2-hydroxy-4-(4-trifluormethylphenyl)but -3-inoylamino]-4-methyl-2,3-benzoxazin-1-one 40b

The racemic mixture which was described in example 40 was separated by preparative chiral HPLC (column Chiralpak AD 250×10 mm) into the enantiomers 40a and 40b.

40a: [α]^{D}₂₀: +20.9° (CHCl₃, 1.06 g/100 ml; λ=589 nM)

40b: [α]^{D}₂₀: −20.6° (CHCl₃, 1.05 g/100 ml; λ=589 nM)

### rac-6-[2-[[3-(1-Hydroxy-1-methylethyl)phenyl]ethynyl]-2-hydroxy-4-methyl-4-phenylpentanoylamino]-4-methyl-2,3-benzoxazin-1-one 41

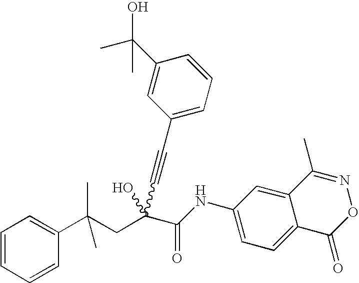

59 μl of 3 molar solution of Methylmagnesium chloride was diluted with 1 ml of pure Tetrahydrofurane. The solution was cooled to −70° C. and a solution of 30 mg of the compound which was described in example 33 in 0,5 ml of pure Tetrahydrofurane was added. After stirring for 2,5 hours at −70° C. the mixture was given to a saturated solution of ammonium chloride. After extracting the mixture with Ethyl acetate the combined organic phases were washed with saturated sodium chloride and dried over sodium sulphate. After column chromatography 16 mg of the product was obtained.

¹H-NMR (ppm, CDCl₃, 400 MHz): 1.46 (3H), 1.53 (6H), 1.62 (3H), 1.80 (1H), 2.55 (3H), 2.65-2.90 (3H), 7.12 (1H), 7.30 (3H), 7.40-7.52 (3H), 7.53 (1H), 7.60 (1H), 8.27 (2H), 8.95 (1H).

The following compound was prepared in analogy to example 7 from the compound which was described in example 14 and 4-Iodobenzylalcohol:

### rac-6-[2-[[4-(Hydroxymethyl)phenyl]ethynyl]-2-hydroxy-4-methyl -4-phenylpentanoylamino]-4-methyl-2,3-benzoxazin-1-one 42

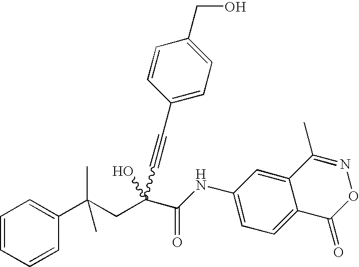

1H-NMR (ppm, CDCl₃, 300 MHz): 1.47 (3H), 1.60 (3H), 1.80 (1H), 2.57 (3H), 2.62-2.83 (3H), 4.68 (2H), 7.13 (1H), 7.25-7.43 (6H), 7.48 (2H), 7.59 (1H), 8.25-8.32 (2H), 8.91 (1H).

The following compound was prepared in analogy to example 7 from the compound which was described in example 14 and 4-Iodobenzylalcohol:

### rac-6-[2-[[3-(Hydroxytnethyl)phenyl]ethinyl]-2-hydroxy-4-methyl -4-phenylpentanoylamino]-4-methyl-2,3-benzoxazin-1-one 43

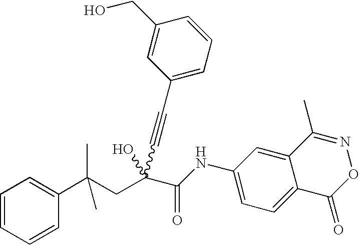

1H-NMR (ppm, CDCl₃, 400 MHz): 1.48 (3H), 1.62 (3H), 1.79 (1H), 2.57 (3H), 2.62-2.80 (3H), 4.68 (2H), 7.15 (1H), 7.25-7.39 (5H), 7.40 (1H), 7.49 (2H), 7.60 (1H), 8.29 (2H), 8.91 (1H).

The following compound was prepared in analogy to example 41 from the compound which was described in example 30 and a solution of Methyl magnesium chloride:

### rac-6-[2-[[4-(1-Hydroxy-1-methylethyl)phenyl]ethynyl]-2-hydroxy-4-methyl -4-phenylpentanoylamino]-4-methyl-2,3-benzoxazin-1-one 44

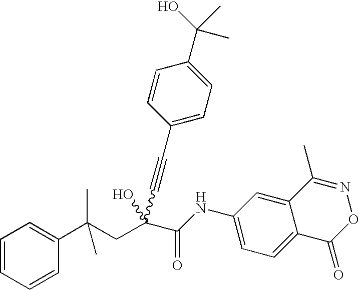

1H-NMR (ppm, CDCl₃, 400 MHz): 1.47 (3H), 1.55 (6H), 1.62 (3H), 1.70 (1H), 2.55 (3H), 2.60-2.80 (3H), 7.14 (1H), 7.28-7.40 (4H), 7.41 (2H), 7.48 (2H), 7.60 (1H), 8.25-8.32 (2H), 8.90 (1H).

The entire disclosures of all applications, patents and publications, cited-herein and of corresponding German application No. 102005030292.0-44, filed Jun. 24, 2005 and U.S. Provisional Application Ser. No. 60/693,403 filed Jun. 24, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I 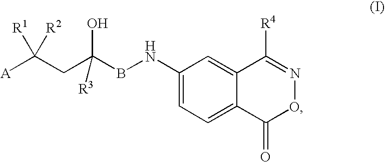
R¹ and R² are independently of one another a hydrogen atom, a linear or nonlinear, branched or unbranched C₁-C₅-alkyl group, further forming together with the C atom of the chain a ring having a total of 3-7 members,
R³ is a radical C≡C—R^{a}, where
R^{a} is a hydrogen or a C₁-C₈-alkyl, C₂-C₈-alkenyl, C₂-C₈-alkynyl, C₃-C₁₀-cycloalkyl, heterocycloalkyl optionally substituted one or more times, identically or differently, by K, or an aryl or heteroaryl optionally substituted one or more times, identically or differently by L,
K is a cyano, halogen, hydroxy, nitro, —C(O)R^{b}, CO₂R^{b}, —O—R^{b}, —S—R^{b}, SO₂NR^{c}R^{d}, —C(O)—NR^{c}R^{d},
—OC(O)—NR^{c}R^{d}, —C═NOR^{b}—NR^{c}R^{d} or C₃-C₁₀-cycloalkyl, heterocycloalkyl optionally substituted one or more times, identically or differently, by M, or aryl or heteroaryl optionally substituted one or more times by L,
L is C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl,
—(CH₂)ₚPO₃(R^{b})₂, —(CH₂)ₚNR^{c}R^{d}, —(CH₂)ₚNR^{e}COR^{b}, —(CH₂)ₚNR^{e}CSR^{b}, —(CH₂)ₚNR^{e}S(O)R^{b}, —(CH₂)ₚNR^{e}S(O)₂R^{b}, —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚS(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{b}, —(CH₂)ₚCONR^{c}R^{d},
—(CH₂)ₚCSNR^{c}R^{d}, —(CH₂)ₚOR^{b}, —(CH₂)ₚSR^{b}, —(CH₂)ₚCR^{b}(OH)—R^{e}, —(CH₂)ₚ—C═NOR^{b},
—O—(CH₂), —O—, —O—(CH₂), —CH₂—, —O—CH═CH— or —(CH₂)ₙ₊₂—, where n is 1 or 2, and the terminal oxygen atoms and/or carbon atoms are linked to directly adjacent ring carbon atoms,
M is C₁-C₆-alkyl or a group —COR^{b}, CO₂R^{b}, —O—R^{b}, or —NR^{c}R^{d}, where
R^{b} is a hydrogen or a C₁-C₆-alkyl, C₂-C₈-alkenyl, C₂-C₈-alkynyl, C₃-C₁₀-cycloalkyl, C₆-C₁₂-aryl or C₁-C₃-perfluoroalkyl and
R^{c} and R^{d} are independently of one another a hydrogen, C₁-C₆-alkyl, C₂-C₈-alkenyl, C₂-C₈-alkynyl, C₃-C₁₀-cycloalkyl, C₆-C₁₂-aryl, C(O)R^{b} or a hydroxy group, where if
R^{c} is a hydroxy group, then R^{d} can only be a hydrogen, a C₁-C₆-alkyl, C₂-C₈-alkenyl, C₂-C₈-alkynyl, C₃-C₁₀-cycloalkyl or C₆-C₁₂-aryl and vice versa, and
R^{e} is a hydrogen, C₁-C₆-alkyl, C₂-C₈-alkenyl, C₂-C₈-alkynyl, C₃-C₁₀-cycloalkyl or C₆-C₁₂-aryl, and p can be a number from 0-6,
or
R³ is a radical C═C—R^{g}R^{h}, where
R^{g} and R^{h} are independently of one another a hydrogen or a C₁-C₈-alkyl, C₂-C₈-alkenyl or C₂-C₈-alkynyl optionally substituted one or more times, identically or differently, by X, in which
X is a cyano, halogen, hydroxy, nitro, —C(O)R^{b}, CO₂R^{b}, —O—R^{b}, —C(O)—NR^{c}R^{d}, —NR^{c}R^{d} with the meanings already mentioned before for R^{b}, R^{c} and R^{d}, and
R⁴ is a hydrogen atom, a methyl or an ethyl group or a partly or completely fluorinated C₁-C₃-alkyl group,
A is a mono- or bicyclic carbocyclic or heterocyclic aromatic ring which may optionally be substituted one or more times by C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkyl, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚPO₃(R^{b})₂, —(CH₂)ₚNR^{c}R^{d}, —(CH₂)ₚNR^{e}COR^{b}, —(CH₂)ₚNR^{e}CSR^{b}, —(CH₂)ₚNR^{e}S(O)R^{b}, —(CH₂) NR^{e}S(O)₂R^{b}, —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, (CH₂)ₚCOR^{b}, —(CH₂)ₚCSR^{b}, —(CH₂)ₚ S(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{b}, —(CH₂)ₚCONR^{c}R^{d}, —(CH₂)ₚCSNR^{c}R^{d}, —(CH₂)ₚOR^{b}, —(CH₂)ₚSR^{b}, —(CH₂)ₚCR^{b}(OH)—R^{d}, —(CH₂)ₚ—C═NOR^{b}, —O—(CH₂), —O—, —O—(CH₂), —CH₂—, —O—CH═CH— or —(CH₂)ₙ₊₂—, where n is 1 or 2, and the terminal oxygen atoms and/or carbon atoms are linked to directly adjacent ring carbon atoms, or
A is a radical —CO₂R^{b}, C(O)NR^{c}R^{d}, COR^{b},
or
A is an alkenyl group —CR⁵═CR⁶R⁷, where
R⁵, R⁶ and R⁷ are identical or different and are independently of one another hydrogen atoms, halogen atoms, aryl radicals or an unsubstituted or partly or completely fluorinated C₁-C₅-alkyl group, or
A is an alkynyl group —C≡CR⁵, with the meaning stated above for R⁵, and
B is a carbonyl or a CH₂ group
and their pharmaceutically acceptable salts.

2. Compounds according to claim 1, in which R¹ and R² are preferably a hydrogen atom, a methyl or ethyl group.

3. Compounds according to claim 1, in which R¹ and R² preferably form together with the C atom of the chain a ring having a total of 3-7 members.

4. Compounds according to claim 1, in which R³ is preferably alkenyl, alkynyl, arylalkynyl, heteroarylalkynyl, cycloalkylalkynyl, heterocycloalkylalkynyl.

5. Compounds according to claim 1, in which R³ is preferably a vinyl, ethynyl, propynyl, butynyl, pentynyl, hexynyl, heptynyl, octynyl, hydroxypropynyl, hydroxybutynyl, 3-hydroxy-3-methylbutynyl, hydroxypentynyl, carboxypropynyl, t-butylcarboxypropynyl, phenylethynyl, (hydroxyphenyl)ethynyl, (methoxyphenyl)ethynyl, (dimethylaminophenyl)ethynyl, (methylphenyl)ethynyl, (cyanophenyl)ethynyl, (trifluoromethyl)ethynyl, (diphenyl)ethynyl, (nitrophenyl)ethynyl, (tert-butylphenyl)ethynyl, (acetylphenyl)ethynyl, (acetoxyphenyl)ethynyl, (carboxyphenyl)ethynyl or a benzylethynyl group.

6. Compounds according to claim 1, in which A is preferably an aromatic ring.

7. Compounds according to claim 1, in which A is preferably a phenyl or naphthyl radical.

8. Compounds according to claim 7, in which A is preferably an unsubstituted or optionally mono- or polysubstituted phenyl radical.

9. Compounds according to claim 8, where the phenyl radical is preferably substituted by one or two halogen atoms or one trifluoromethyl group.

10. Compounds according to claim 9, in which the halogen atoms are preferably chlorine and/or fluorine.

11. Compounds according to claim 1, in which A is preferably a phenyl ring substituted by —O—(CH₂)ₙ—O— or —O—(CH₂)ₙ—CH₂—, where the respectively directly adjacent ring carbon atoms are linked.

12. Compounds according to claim 1, in which R⁴ is a hydrogen atom, a methyl or a trifluoromethyl radical.

13. Compounds according to claim 1, namely

| | | |
|---|---|---|
| | Racemic or | |
| No. | enantiomer | R3 |

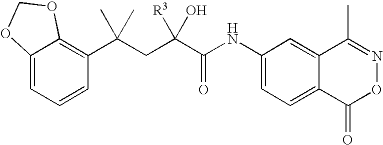
| | | |
|---|---|---|
| 1 2 3 | rac −+ | 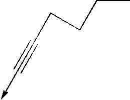 |
| 4 5 6 | rac +− | 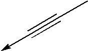 |
| 7 8 9 | rac +− | 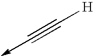 |
| 10 11 12 | rac +− | 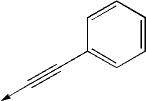 |
| 13 14 15 | rac +− | 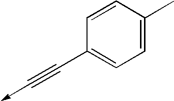 |
| 16 17 18 | rac +− | 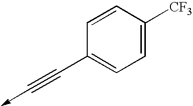 |
| 19 20 21 | rac +− | 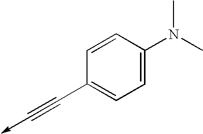 |
| 22 23 24 | rac +− | 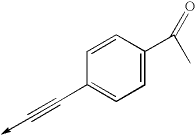 |
| 25 26 27 | rac +− | 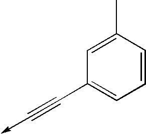 |
| 28 29 30 | rac +− | 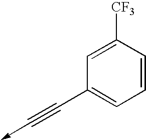 |
| 31 32 33 | rac +− | 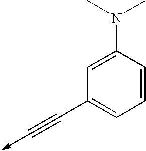 |
| 34 35 36 | rac +− | 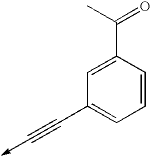 |
| 37 38 39 | rac +− | 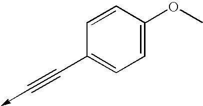 |
| 40 41 42 | rac +− | 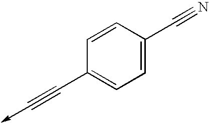 |
| 43 44 45 | rac +− | 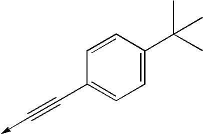 |
| 46 47 48 | rac +− | 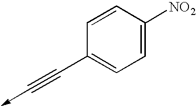 |
| 49 50 51 | rac +− | 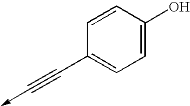 |

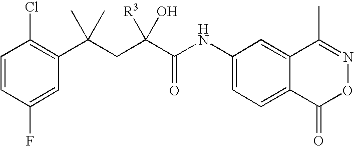
| | | |
|---|---|---|
| 52 53 54 | rac −+ | 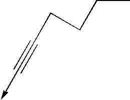 |
| 55 56 57 | rac +− | 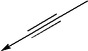 |
| 58 59 60 | rac +− | 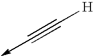 |
| 61 62 63 | rac +− | 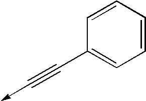 |
| 64 65 66 | rac +− | 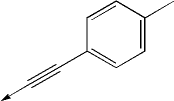 |
| 67 68 69 | rac +− | 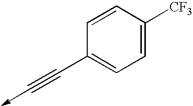 |
| 70 71 72 | rac +− | 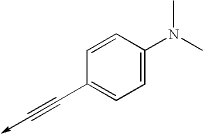 |
| 73 74 75 | rac +− | 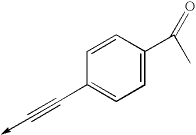 |
| 76 77 78 | rac +− | 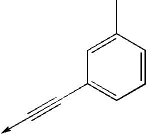 |
| 79 80 81 | rac +− | 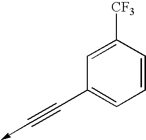 |
| 82 83 84 | rac +− | 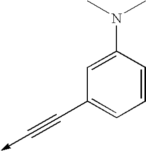 |
| 85 86 87 | rac +− | 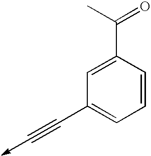 |
| 88 89 90 | rac +− | 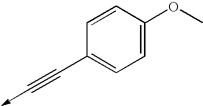 |
| 91 92 93 | rac +− | 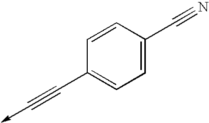 |
| 94 95 96 | rac +− | 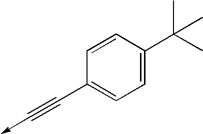 |
| 97 98 99 | rac +− | 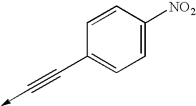 |
| 100 191 102 | rac +− | 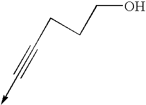 |
| 103 104 105 | rac +− | 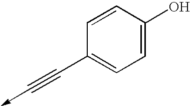 |

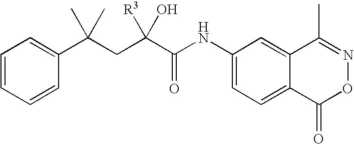
| | | |
|---|---|---|
| 106 107 108 | rac −+ | 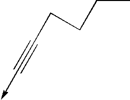 |
| 109 110 111 | rac +− | 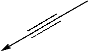 |
| 112 113 114 | rac +− | 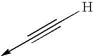 |
| 115 116 117 | rac +− | 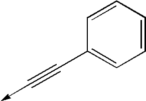 |
| 118 119 120 | rac +− | 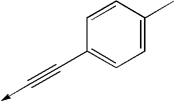 |
| 121 122 123 | rac +− | 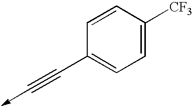 |
| 124 125 126 | rac +− | 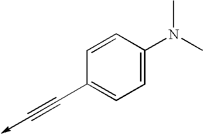 |
| 127 128 129 | rac +− | 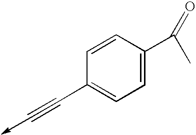 |
| 130 131 132 | rac +− | 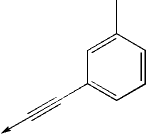 |
| 133 134 135 | rac +− | 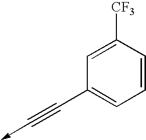 |
| 136 137 138 | rac +− | 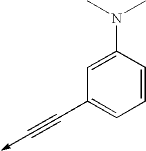 |
| 139 140 141 | rac +− | 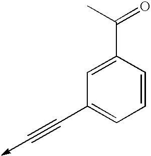 |
| 142 143 144 | rac +− | 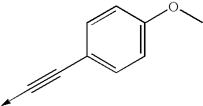 |
| 145 146 147 | rac +− | 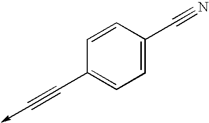 |
| 148 149 150 | rac +− | 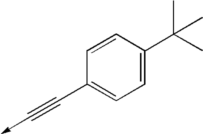 |
| 151 152 153 | rac +− | 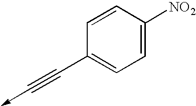 |
| 154 155 156 | rac +− | 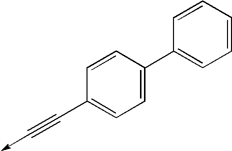 |
| 157 158 159 | rac +− | 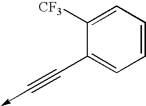 |
| 160 161 162 | rac +− | 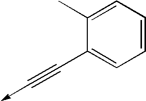 |
| 163 164 165 | rac +− | 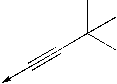 |
| 166 167 168 | rac +− | 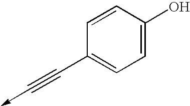 |

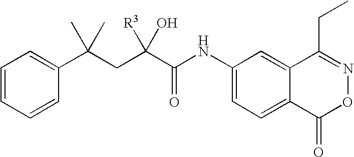
| | | |
|---|---|---|
| 169 170 171 | rac −+ | 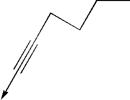 |
| 172 173 174 | rac +− | 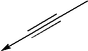 |
| 175 176 177 | rac +− | 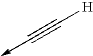 |
| 178 179 180 | rac +− | 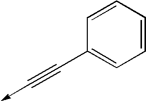 |
| 181 182 183 | rac +− | 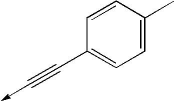 |
| 184 185 186 | rac +− | 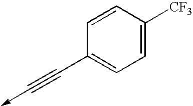 |
| 187 188 189 | rac +− | 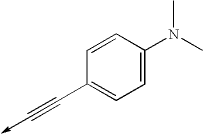 |
| 190 191 192 | rac +− | 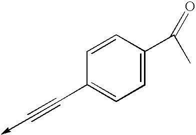 |
| 193 194 195 | rac +− | 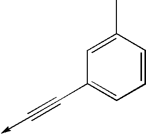 |
| 196 197 198 | rac +− | 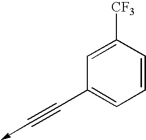 |
| 199 200 201 | rac +− | 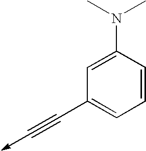 |
| 202 203 204 | rac +− | 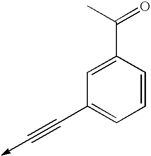 |
| 205 206 207 | rac +− | 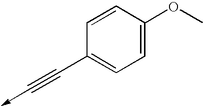 |
| 208 209 210 | rac +− | 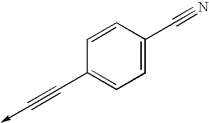 |
| 211 212 213 | rac +− | 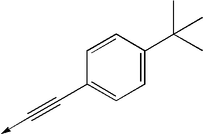 |
| 214 215 216 | rac +− | 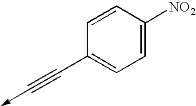 |
| 217 218 219 | rac +− | 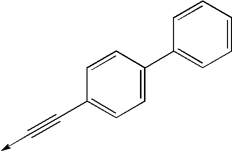 |
| 220 221 222 | rac +− | 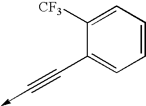 |
| 223 224 225 | rac +− | 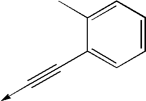 |
| 226 227 228 | rac +− | 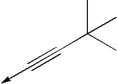 |
| 229 230 231 | rac +− | 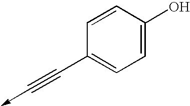 |

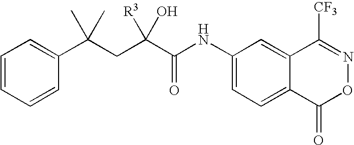
| | | |
|---|---|---|
| 232 233 234 | rac −+ | 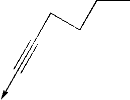 |
| 235 236 237 | rac +− | 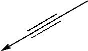 |
| 238 239 240 | rac +− | 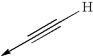 |
| 241 242 243 | rac +− | 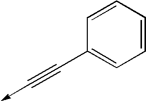 |
| 244 245 246 | rac +− | 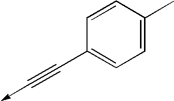 |
| 247 248 249 | rac +− | 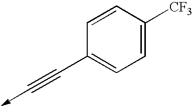 |
| 250 251 252 | rac +− | 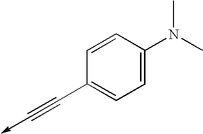 |
| 253 254 255 | rac +− | 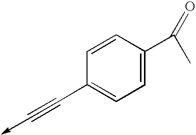 |
| 256 257 258 | rac +− | 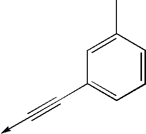 |
| 259 260 261 | rac +− | 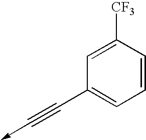 |
| 262 263 264 | rac +− | 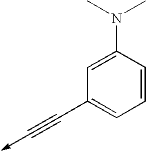 |
| 265 266 267 | rac +− | 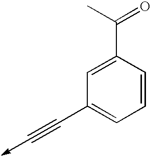 |
| 268 269 270 | rac +− | 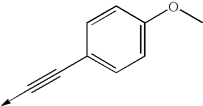 |
| 271 272 273 | rac +− | 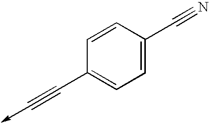 |
| 274 275 276 | rac +− | 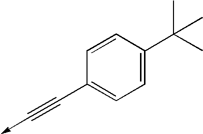 |
| 277 278 279 | rac +− | 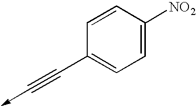 |
| 280 281 282 | rac +− | 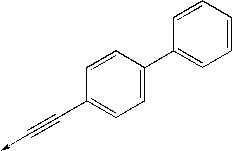 |
| 283 284 285 | rac +− | 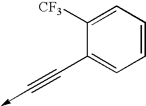 |
| 286 287 288 | rac +− | 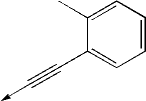 |
| 289 290 291 | rac +− | 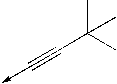 |
| 292 293 294 | rac +− | 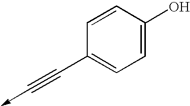 |

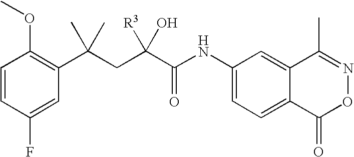
| | | |
|---|---|---|
| 295 296 297 | rac −+ | 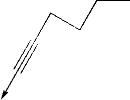 |
| 298 299 300 | rac +− | 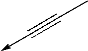 |
| 301 302 303 | rac +− | 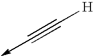 |
| 304 305 306 | rac +− | 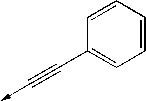 |
| 307 308 309 | rac +− | 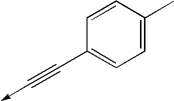 |
| 310 311 312 | rac +− | 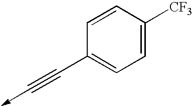 |
| 313 314 315 | rac +− | 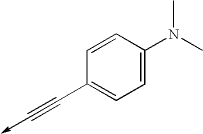 |
| 316 317 317 | rac +− | 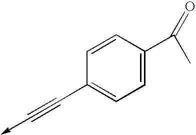 |
| 319 320 321 | rac +− | 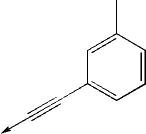 |
| 322 323 324 | rac +− | 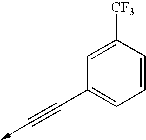 |
| 325 326 327 | rac +− | 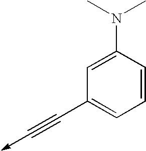 |
| 328 329 330 | rac +− | 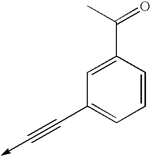 |
| 331 332 333 | rac +− | 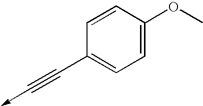 |
| 334 335 336 | rac +− | 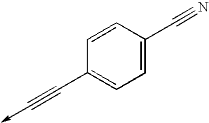 |
| 337 338 339 | rac +− | 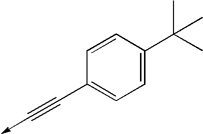 |
| 340 341 342 | rac +− | 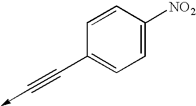 |
| 343 344 345 | rac +− | 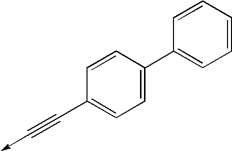 |
| 346 347 348 | rac +− | 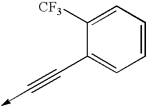 |
| 349 350 351 | rac +− | 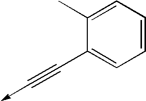 |
| 352 353 354 | rac +− | 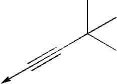 |
| 355 356 357 | rac +− | 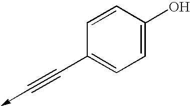 |

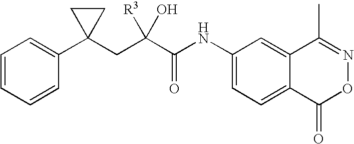
| | | |
|---|---|---|
| 358 359 360 | rac −+ | 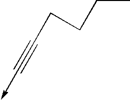 |
| 361 362 363 | rac +− | 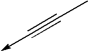 |
| 364 365 366 | rac +− | 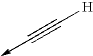 |
| 367 368 369 | rac +− | 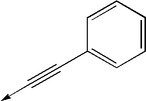 |
| 370 371 372 | rac +− | 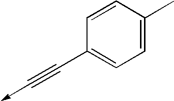 |
| 373 374 375 | rac +− | 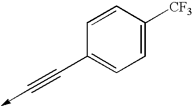 |
| 376 377 378 | rac +− | 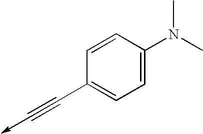 |
| 379 380 381 | rac +− | 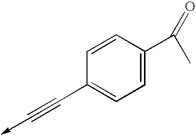 |
| 382 383 384 | rac +− | 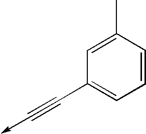 |
| 385 386 387 | rac +− | 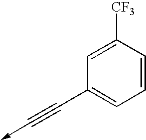 |
| 388 389 390 | rac +− | 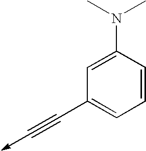 |
| 391 392 393 | rac +− | 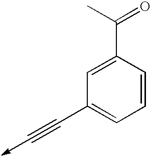 |
| 394 395 396 | rac +− | 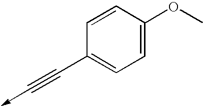 |
| 397 398 399 | rac +− | 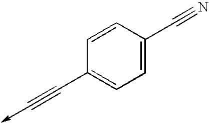 |
| 400 401 402 | rac +− | 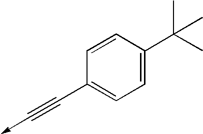 |
| 403 404 405 | rac +− | 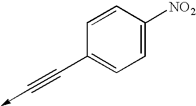 |
| 406 407 408 | rac +− | 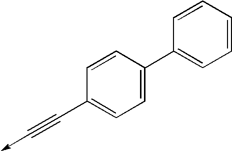 |
| 409 410 411 | rac +− | 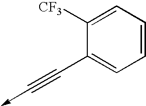 |
| 412 413 414 | rac +− | 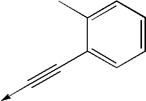 |
| 415 416 417 | rac +− | 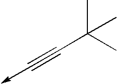 |
| 418 419 420 | rac +− | 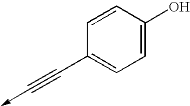 |

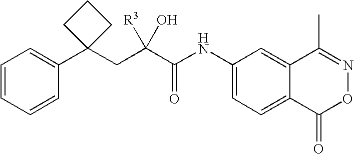
| | | |
|---|---|---|
| 421 422 423 | rac −+ | 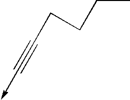 |
| 424 425 426 | rac +− | 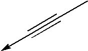 |
| 427 428 429 | rac +− | 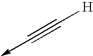 |
| 430 431 432 | rac +− | 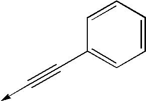 |
| 433 434 435 | rac +− | 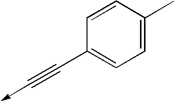 |
| 436 437 438 | rac +− | 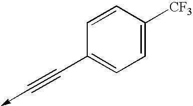 |
| 439 440 441 | rac +− | 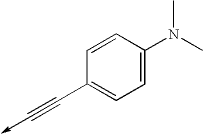 |
| 442 443 444 | rac +− | 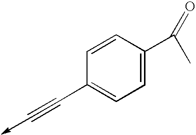 |
| 445 446 447 | rac +− | 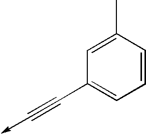 |
| 448 449 450 | rac +− | 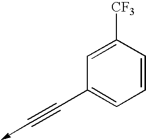 |
| 451 452 453 | rac +− | 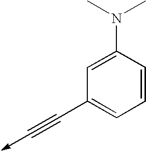 |
| 454 455 456 | rac +− | 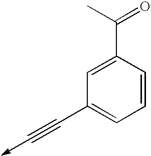 |
| 457 458 459 | rac +− | 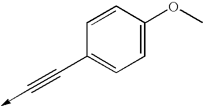 |
| 460 461 462 | rac +− | 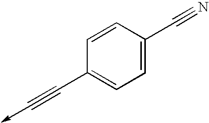 |
| 463 464 465 | rac +− | 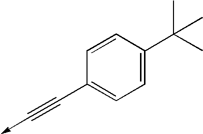 |
| 466 467 468 | rac +− | 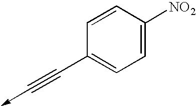 |
| 469 470 471 | rac +− | 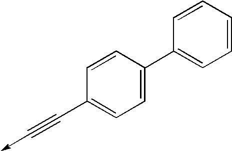 |
| 472 473 474 | rac +− | 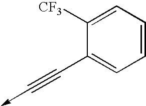 |
| 475 476 477 | rac +− | 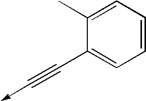 |
| 478 479 480 | rac +− | 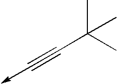 |
| 481 482 483 | rac +− | 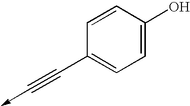 |

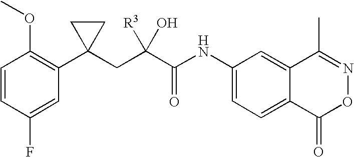
| | | |
|---|---|---|
| 484 485 486 | rac −+ | 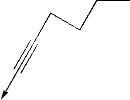 |
| 487 488 489 | rac +− | 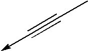 |
| 490 491 492 | rac +− | 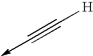 |
| 493 494 495 | rac +− | 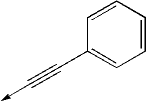 |
| 496 497 498 | rac +− | 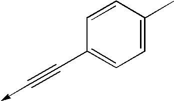 |
| 499 500 501 | rac +− | 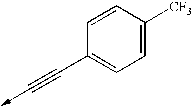 |
| 502 503 504 | rac +− | 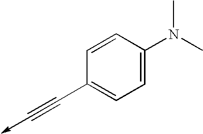 |
| 505 506 507 | rac +− | 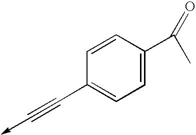 |
| 508 509 510 | rac +− | 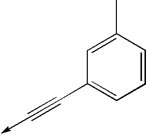 |
| 511 512 513 | rac +− | 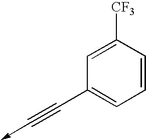 |
| 514 515 516 | rac +− | 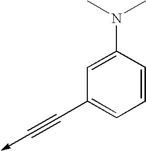 |
| 517 518 519 | rac +− | 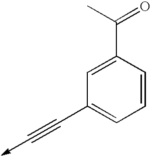 |
| 520 521 522 | rac +− | 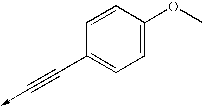 |
| 523 524 525 | rac +− | 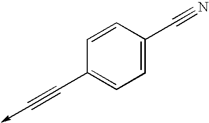 |
| 526 527 528 | rac +− | 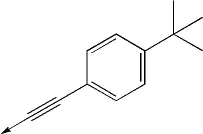 |
| 529 530 531 | rac +− | 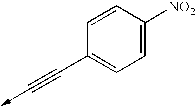 |
| 532 533 534 | rac +− | 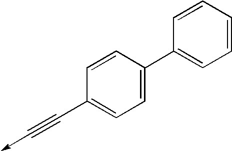 |
| 535 536 537 | rac +− | 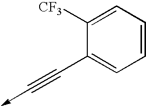 |
| 538 539 540 | rac +− | 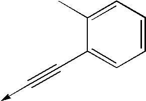 |
| 541 542 543 | rac +− | 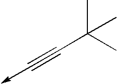 |
| 544 545 546 | rac +− | 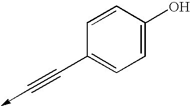 |

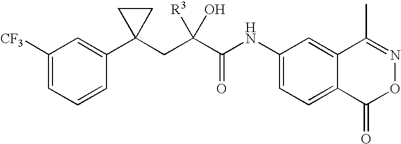
| | | |
|---|---|---|
| 547 548 549 | rac −+ | 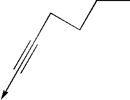 |
| 550 551 552 | rac +− | 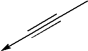 |
| 553 554 555 | rac +− | 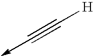 |
| 556 557 558 | rac +− | 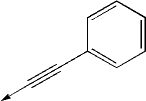 |
| 559 560 561 | rac +− | 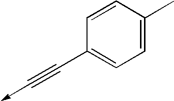 |
| 562 563 564 | rac +− | 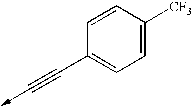 |
| 565 566 567 | rac +− | 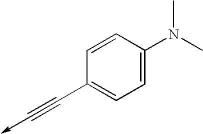 |
| 568 569 570 | rac +− | 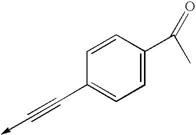 |
| 571 572 573 | rac +− | 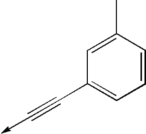 |
| 574 575 576 | rac +− | 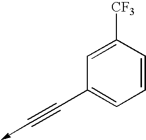 |
| 577 578 579 | rac +− | 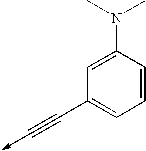 |
| 580 581 582 | rac +− | 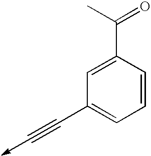 |
| 583 584 585 | rac +− | 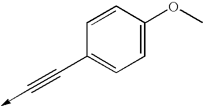 |
| 586 587 588 | rac +− | 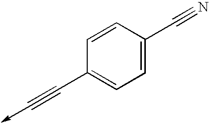 |
| 589 590 591 | rac +− | 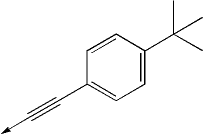 |
| 592 593 594 | rac +− | 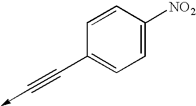 |
| 595 596 597 | rac +− | 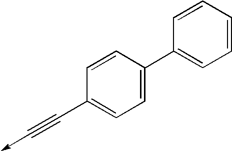 |
| 598 599 600 | rac +− | 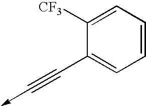 |
| 601 602 603 | rac +− | 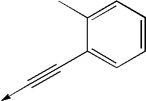 |
| 604 605 606 | rac +− | 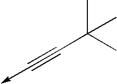 |
| 607 608 609 | rac +− | 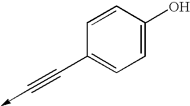 |

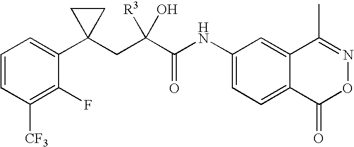
| | | |
|---|---|---|
| 610 611 612 | rac −+ | 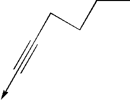 |
| 613 614 615 | rac +− | 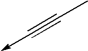 |
| 616 617 618 | rac +− | 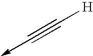 |
| 619 620 621 | rac +− | 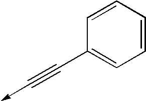 |
| 622 623 624 | rac +− | 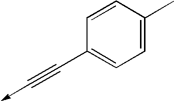 |
| 625 626 627 | rac +− | 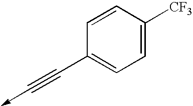 |
| 628 629 630 | rac +− | 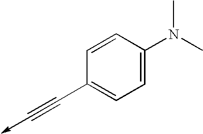 |
| 631 632 633 | rac +− | 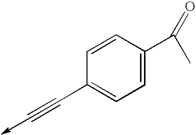 |
| 634 635 636 | rac +− | 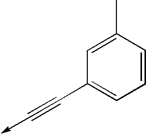 |
| 637 638 639 | rac +− | 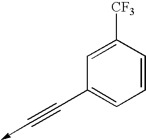 |
| 640 641 642 | rac +− | 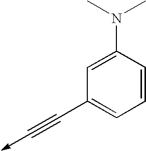 |
| 643 644 645 | rac +− | 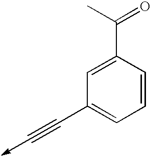 |
| 646 647 648 | rac +− | 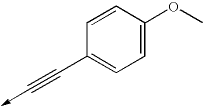 |
| 649 650 651 | rac +− | 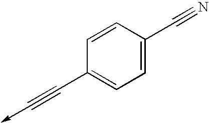 |
| 652 653 654 | rac +− | 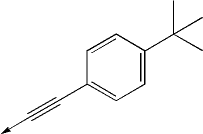 |
| 655 656 657 | rac +− | 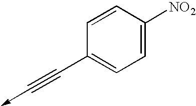 |
| 658 659 660 | rac +− | 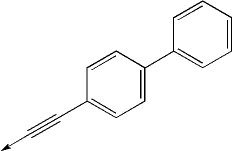 |
| 661 662 663 | rac +− | 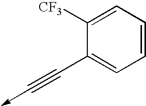 |
| 664 665 666 | rac +− | 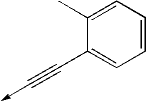 |
| 667 668 669 | rac +− | 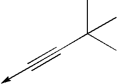 |
| 670 671 672 | rac +− | 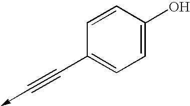 |

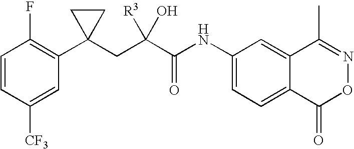
| | | |
|---|---|---|
| 673 674 675 | rac −+ | 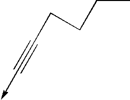 |
| 676 677 678 | rac +− | 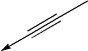 |
| 679 680 681 | rac +− | 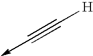 |
| 682 683 684 | rac +− | 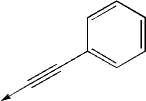 |
| 685 686 687 | rac +− | 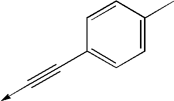 |
| 688 689 690 | rac +− | 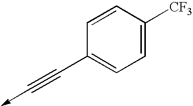 |
| 691 692 693 | rac +− | 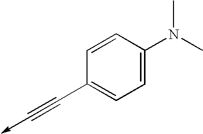 |
| 694 695 696 | rac +− | 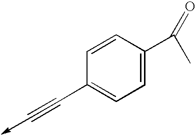 |
| 697 698 699 | rac +− | 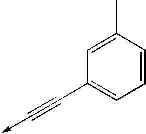 |
| 700 701 702 | rac +− | 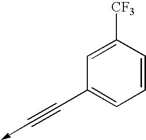 |
| 703 704 705 | rac +− | 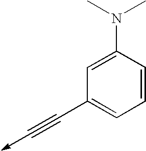 |
| 706 707 708 | rac +− | 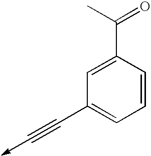 |
| 709 710 711 | rac +− | 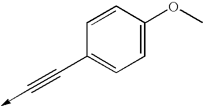 |
| 712 713 714 | rac +− | 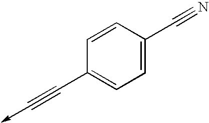 |
| 715 716 717 | rac +− | 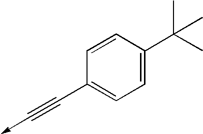 |
| 718 719 720 | rac +− | 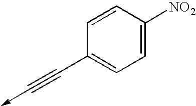 |
| 721 722 723 | rac +− | 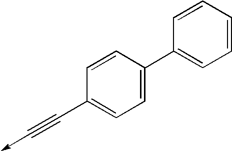 |
| 724 725 726 | rac +− | 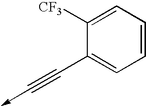 |
| 727 728 729 | rac +− | 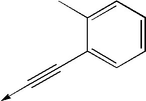 |
| 730 731 732 | rac +− | 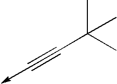 |
| 733 734 735 | rac +− | 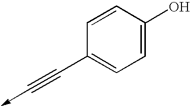 |

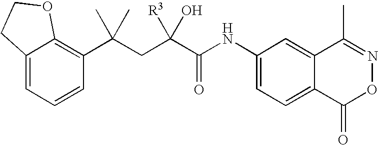
| | | |
|---|---|---|
| 736 737 738 | rac −+ | 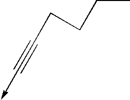 |
| 739 740 741 | rac +− | 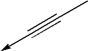 |
| 742 743 744 | rac +− | 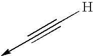 |
| 745 746 747 | rac +− | 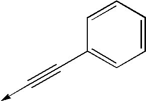 |
| 748 749 750 | rac +− | 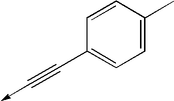 |
| 751 752 753 | rac +− | 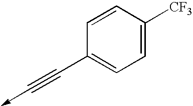 |
| 754 755 756 | rac +− | 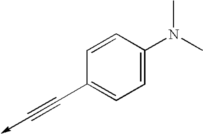 |
| 757 758 759 | rac +− | 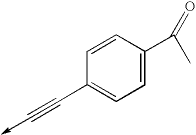 |
| 760 761 762 | rac +− | 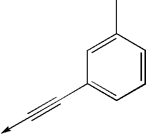 |
| 763 764 765 | rac +− | 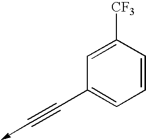 |
| 766 767 768 | rac +− | 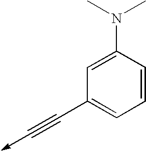 |
| 769 770 771 | rac +− | 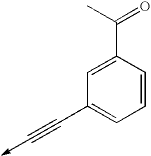 |
| 772 773 774 | rac +− | 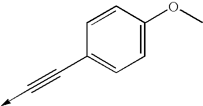 |
| 775 776 777 | rac +− | 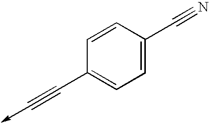 |
| 778 779 780 | rac +− | 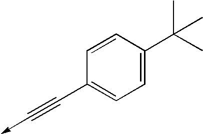 |
| 781 782 783 | rac +− | 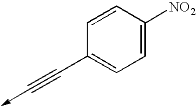 |
| 784 785 786 | rac +− | 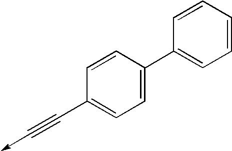 |
| 787 788 789 | rac +− | 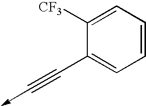 |
| 790 791 792 | rac +− | 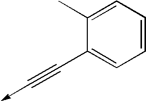 |
| 793 794 795 | rac +− | 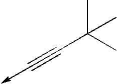 |
| 796 797 798 | rac +− | 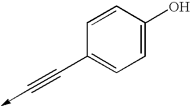 |

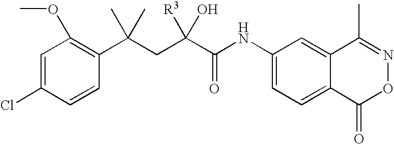
| | | |
|---|---|---|
| 799 800 801 | rac −+ | 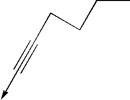 |
| 802 803 804 | rac +− | 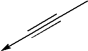 |
| 805 806 807 | rac +− | 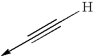 |
| 808 809 810 | rac +− | 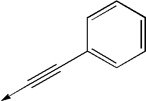 |
| 811 812 813 | rac +− | 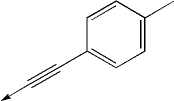 |
| 814 815 816 | rac +− | 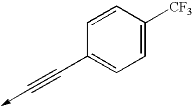 |
| 817 818 819 | rac +− | 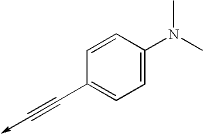 |
| 820 821 822 | rac +− | 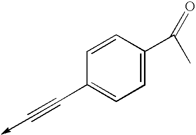 |
| 823 824 825 | rac +− | 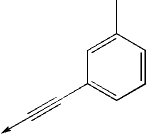 |
| 826 827 828 | rac +− | 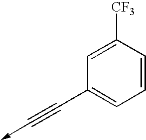 |
| 829 830 831 | rac +− | 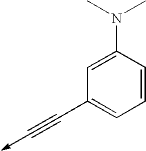 |
| 832 833 834 | rac +− | 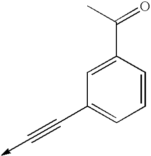 |
| 835 836 837 | rac +− | 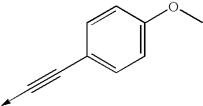 |
| 838 839 840 | rac +− | 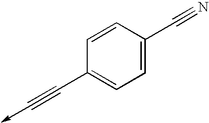 |
| 841 842 843 | rac +− | 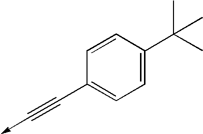 |
| 844 845 846 | rac +− | 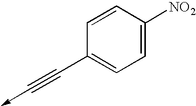 |
| 847 848 849 | rac +− | 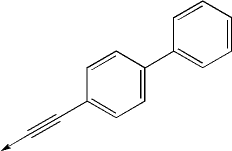 |
| 850 851 852 | rac +− | 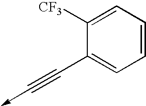 |
| 853 854 855 | rac +− | 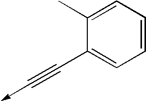 |
| 856 857 858 | rac +− | 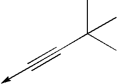 |
| 859 860 861 | rac +− | 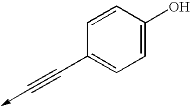 |

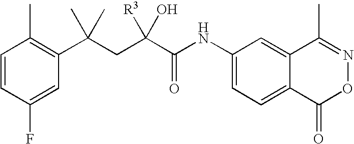
| | | |
|---|---|---|
| 862 863 864 | rac −+ | 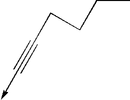 |
| 865 866 867 | rac +− | 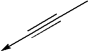 |
| 868 869 870 | rac +− | 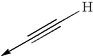 |
| 871 872 873 | rac +− | 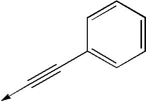 |
| 874 875 876 | rac +− | 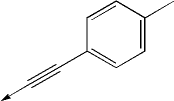 |
| 877 878 879 | rac +− | 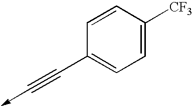 |
| 880 881 882 | rac +− | 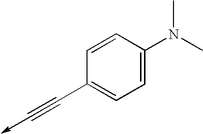 |
| 883 884 885 | rac +− | 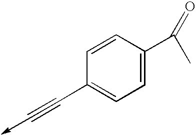 |
| 886 887 888 | rac +− | 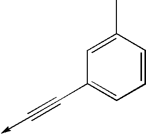 |
| 889 890 891 | rac +− | 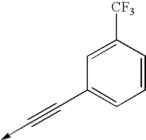 |
| 892 893 894 | rac +− | 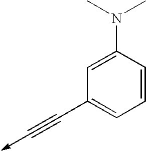 |
| 895 896 897 | rac +− | 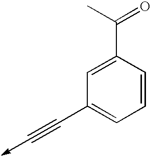 |
| 898 899 900 | rac +− | 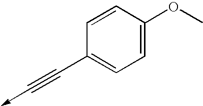 |
| 901 902 903 | rac +− | 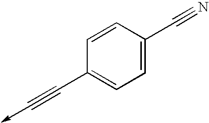 |
| 904 905 906 | rac +− | 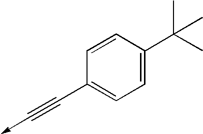 |
| 907 908 909 | rac +− | 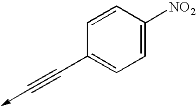 |
| 910 911 912 | rac +− | 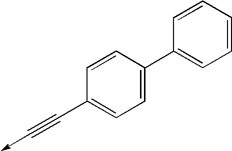 |
| 913 914 915 | rac +− | 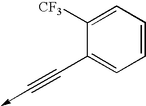 |
| 916 917 918 | rac +− | 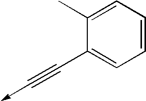 |
| 919 920 921 | rac +− | 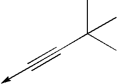 |
| 922 923 924 | rac +− | 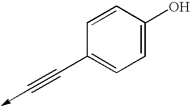 |

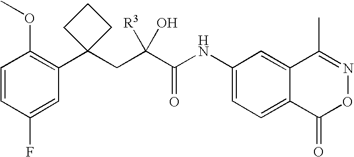
| | | |
|---|---|---|
| 925 926 927 | rac −+ | 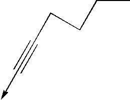 |
| 928 929 930 | rac +− | 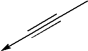 |
| 931 932 933 | rac +− | 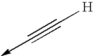 |
| 934 935 936 | rac +− | 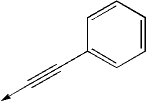 |
| 937 938 939 | rac +− | 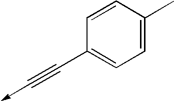 |
| 940 941 942 | rac +− | 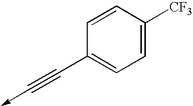 |
| 943 944 945 | rac +− | 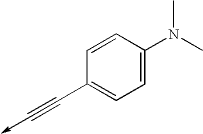 |
| 946 947 948 | rac +− | 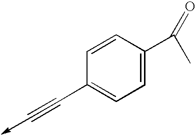 |
| 949 950 951 | rac +− | 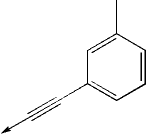 |
| 952 953 954 | rac +− | 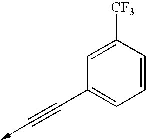 |
| 955 956 957 | rac +− | 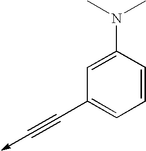 |
| 958 959 960 | rac +− | 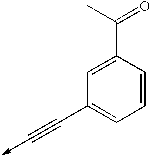 |
| 961 962 963 | rac +− | 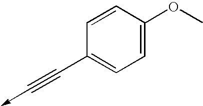 |
| 964 965 966 | rac +− | 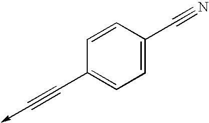 |
| 967 968 969 | rac +− | 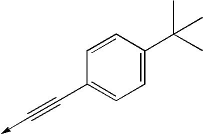 |
| 970 971 972 | rac +− | 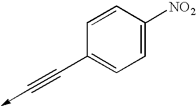 |
| 973 974 975 | rac +− | 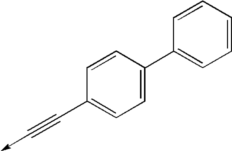 |
| 976 977 978 | rac +− | 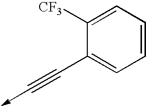 |
| 979 980 981 | rac +− | 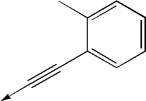 |
| 982 983 984 | rac +− | 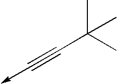 |
| 985 986 987 | rac +− | 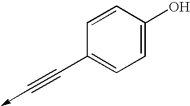 |

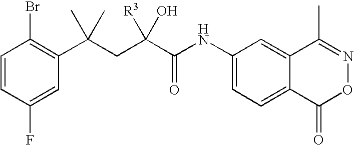
| | | |
|---|---|---|
| 988 989 990 | rac −+ | 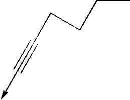 |
| 991 992 993 | rac +− | 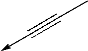 |
| 994 995 996 | rac +− | 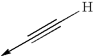 |
| 997 998 999 | rac +− | 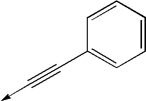 |
| 1000 1001 1002 | rac +− | 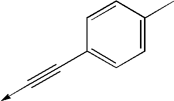 |
| 1003 1004 1005 | rac +− | 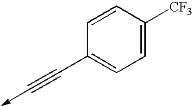 |
| 1006 1007 1008 | rac +− | 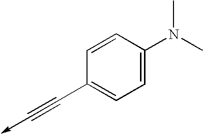 |
| 1009 1010 1011 | rac +− | 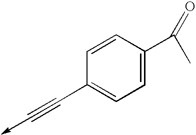 |
| 1012 1013 1014 | rac +− | 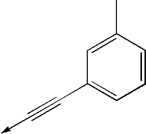 |
| 1015 1016 1017 | rac +− | 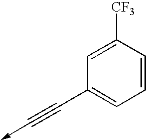 |
| 1018 1019 1020 | rac +− | 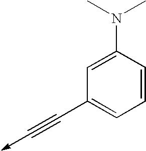 |
| 1021 1022 1023 | rac +− | 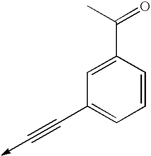 |
| 1024 1025 1026 | rac +− | 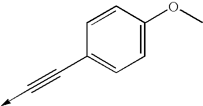 |
| 1027 1028 1029 | rac +− | 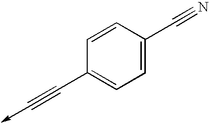 |
| 1030 1031 1032 | rac +− | 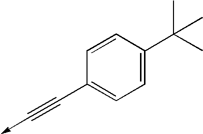 |
| 1033 1034 1035 | rac +− | 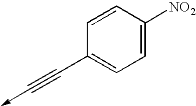 |
| 1036 1037 1038 | rac +− | 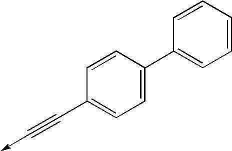 |
| 1039 1040 1041 | rac +− | 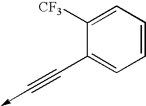 |
| 1042 1043 1044 | rac +− | 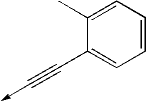 |
| 1045 1046 1047 | rac +− | 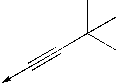 |
| 1048 1049 1050 | rac +− | 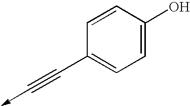 |

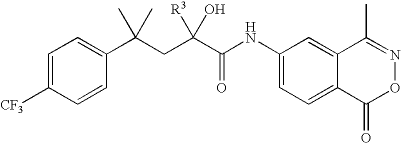
| | | |
|---|---|---|
| 1051 1052 1053 | rac −+ | 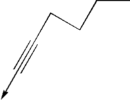 |
| 1054 1055 1056 | rac +− | 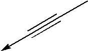 |
| 1057 1058 1059 | rac +− | 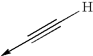 |
| 1060 1061 1062 | rac +− | 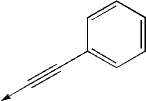 |
| 1063 1064 1065 | rac +− | 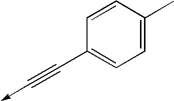 |
| 1066 1067 1068 | rac +− | 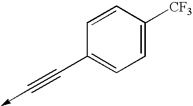 |
| 1069 1070 1071 | rac +− | 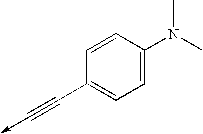 |
| 1072 1073 1074 | rac +− | 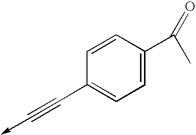 |
| 1075 1076 1077 | rac +− | 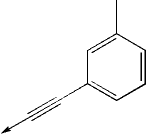 |
| 1078 1079 1080 | rac +− | 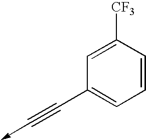 |
| 1081 1082 1083 | rac +− | 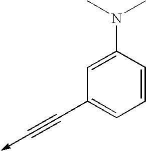 |
| 1084 1085 1086 | rac +− | 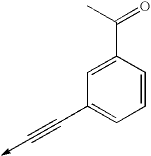 |
| 1087 1088 1089 | rac +− | 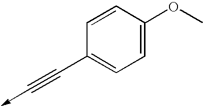 |
| 1090 1091 1092 | rac +− | 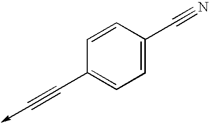 |
| 1093 1094 1095 | rac +− | 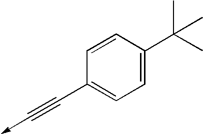 |
| 1096 1097 1098 | rac +− | 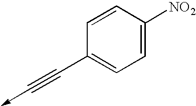 |
| 1099 1100 1101 | rac +− | 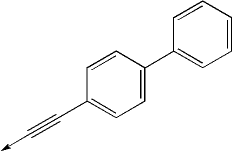 |
| 1102 1103 1104 | rac +− | 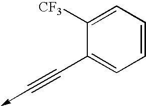 |
| 1105 1106 1107 | rac +− | 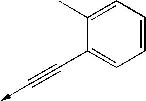 |
| 1108 1109 1110 | rac +− | 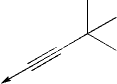 |
| 1111 1112 1113 | rac +− | 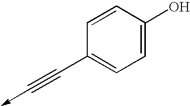 |

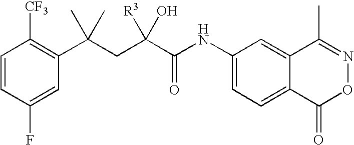
| | | |
|---|---|---|
| 1114 1115 1116 | rac −+ | 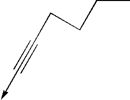 |
| 1117 1118 1119 | rac +− | 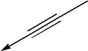 |
| 1120 1121 1122 | rac +− | 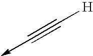 |
| 1123 1124 1125 | rac +− | 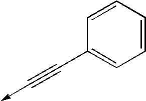 |
| 1126 1127 1128 | rac +− | 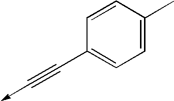 |
| 1129 1130 1131 | rac +− | 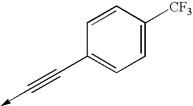 |
| 1132 1133 1134 | rac +− | 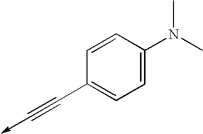 |
| 1135 1136 1137 | rac +− | 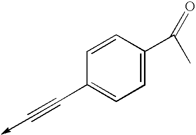 |
| 1138 1139 1140 | rac +− | 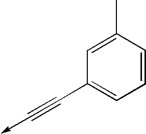 |
| 1141 1142 1143 | rac +− | 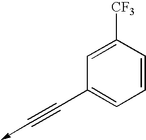 |
| 1144 1145 1146 | rac +− | 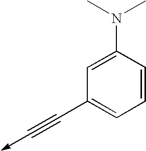 |
| 1147 1148 1149 | rac +− | 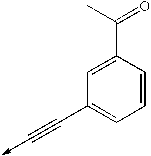 |
| 1150 1151 1152 | rac +− | 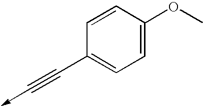 |
| 1153 1154 1155 | rac +− | 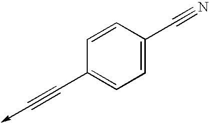 |
| 1156 1157 1158 | rac +− | 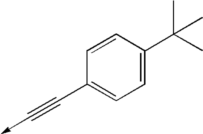 |
| 1159 1160 1161 | rac +− | 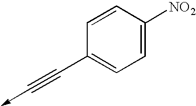 |
| 1162 1163 1164 | rac +− | 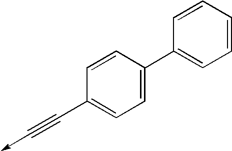 |
| 1165 1166 1167 | rac +− | 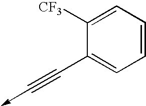 |
| 1168 1169 1170 | rac +− | 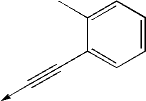 |
| 1171 1172 1173 | rac +− | 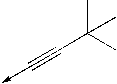 |
| 1174 1175 1176 | rac +− | 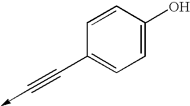 |

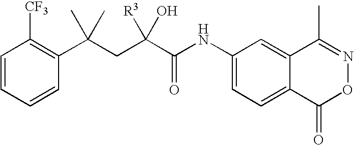
| | | |
|---|---|---|
| 1177 1178 1179 | rac −+ | 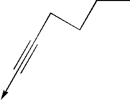 |
| 1180 1181 1182 | rac +− | 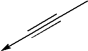 |
| 1183 1184 1185 | rac +− | 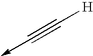 |
| 1186 1187 1188 | rac +− | 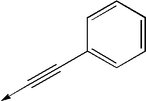 |
| 1189 1190 1191 | rac +− | 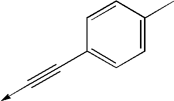 |
| 1192 1193 1194 | rac +− | 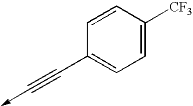 |
| 1195 1196 1197 | rac +− | 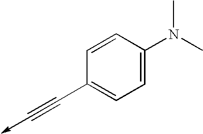 |
| 1198 1199 1200 | rac +− | 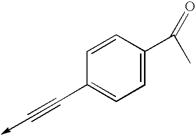 |
| 1202 1202 1203 | rac +− | 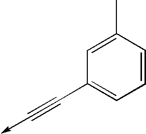 |
| 1204 1205 1206 | rac +− | 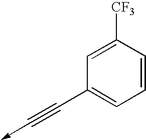 |
| 1207 1208 1209 | rac +− | 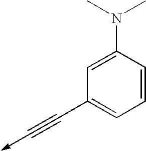 |
| 1210 1211 1212 | rac +− | 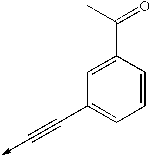 |
| 1213 1214 1215 | rac +− | 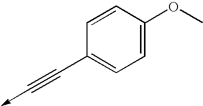 |
| 1216 1217 1218 | rac +− | 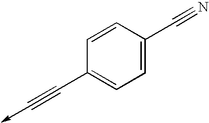 |
| 1219 1220 1221 | rac +− | 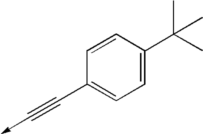 |
| 1222 1223 1224 | rac +− | 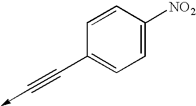 |
| 1225 1226 1227 | rac +− | 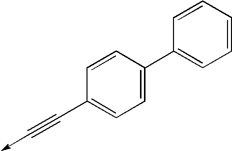 |
| 1228 1229 1230 | rac +− | 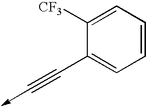 |
| 1231 1232 1233 | rac +− | 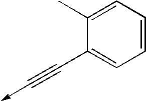 |
| 1234 1235 1236 | rac +− | 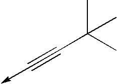 |
| 1237 1238 1239 | rac +− | 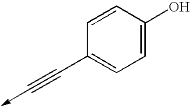 |

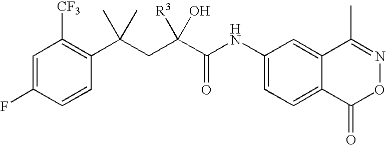
| | | |
|---|---|---|
| 1240 1241 1242 | rac −+ | 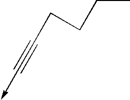 |
| 1243 1244 1245 | rac +− | 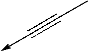 |
| 1246 1247 1248 | rac +− | 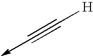 |
| 1249 1250 1251 | rac +− | 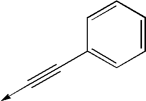 |
| 1252 1253 1254 | rac +− | 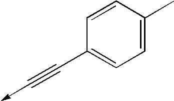 |
| 1255 1256 1257 | rac +− | 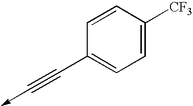 |
| 1258 1259 1260 | rac +− | 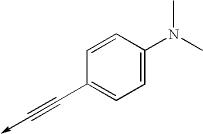 |
| 1261 1262 1263 | rac +− | 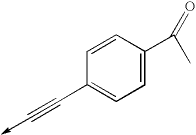 |
| 1264 1265 1266 | rac +− | 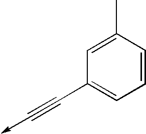 |
| 1267 1268 1269 | rac +− | 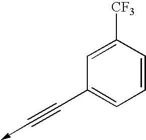 |
| 1270 1271 1272 | rac +− | 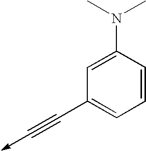 |
| 1273 1274 1275 | rac +− | 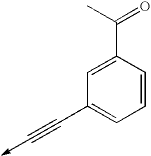 |
| 1276 1277 1278 | rac +− | 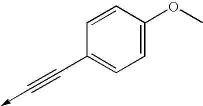 |
| 1279 1280 1281 | rac +− | 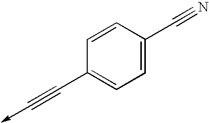 |
| 1282 1283 1284 | rac +− | 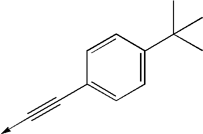 |
| 1285 1286 1287 | rac +− | 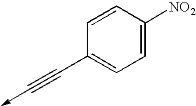 |
| 1288 1289 1290 | rac +− | 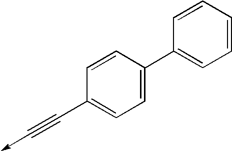 |
| 1291 1292 1293 | rac +− | 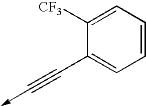 |
| 1294 1295 1296 | rac +− | 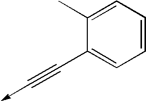 |
| 1297 1298 1299 | rac +− | 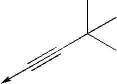 |
| 1300 1301 1302 | rac +− | 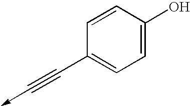 |

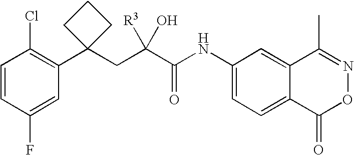
| | | |
|---|---|---|
| 1303 1304 1305 | rac −+ | 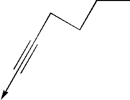 |
| 1306 1307 1308 | rac +− | 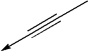 |
| 1309 1310 1311 | rac +− | 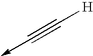 |
| 1312 1313 1314 | rac +− | 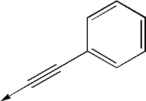 |
| 1315 1316 1317 | rac +− | 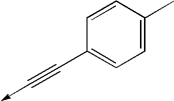 |
| 1318 1319 1320 | rac +− | 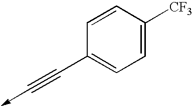 |
| 1321 1322 1323 | rac +− | 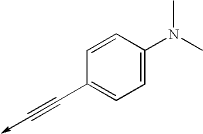 |
| 1324 1325 1326 | rac +− | 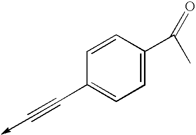 |
| 1327 1328 1329 | rac +− | 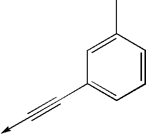 |
| 1330 1331 1332 | rac +− | 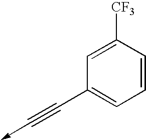 |
| 1333 1334 1335 | rac +− | 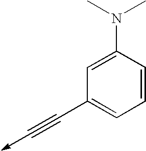 |
| 1336 1337 1338 | rac +− | 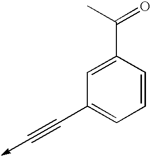 |
| 1339 1340 1341 | rac +− | 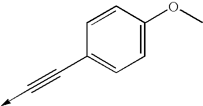 |
| 1342 1343 1344 | rac +− | 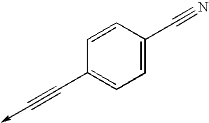 |
| 1345 1346 1347 | rac +− | 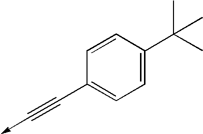 |
| 1348 1349 1350 | rac +− | 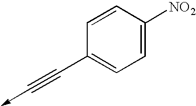 |
| 1351 1352 1353 | rac +− | 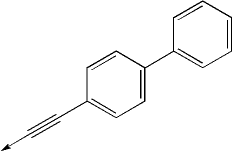 |
| 1354 1355 1356 | rac +− | 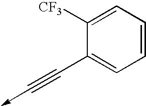 |
| 1357 1358 1359 | rac +− | 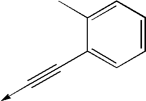 |
| 1360 1361 1362 | rac +− | 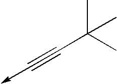 |
| 1362 1364 1365 | rac +− | 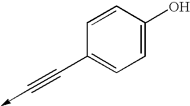 |

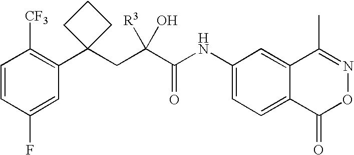
| | | |
|---|---|---|
| 1366 1367 1368 | rac −+ | 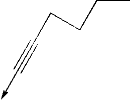 |
| 1369 1370 1371 | rac +− | 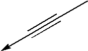 |
| 1372 1373 1374 | rac +− | 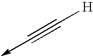 |
| 1375 1376 1377 | rac +− | 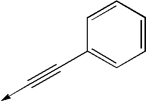 |
| 1378 1379 1380 | rac +− | 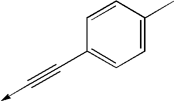 |
| 1381 1382 1383 | rac +− | 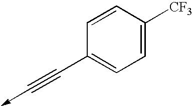 |
| 1384 1385 1386 | rac +− | 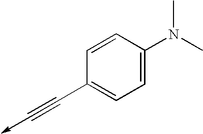 |
| 1387 1388 1389 | rac +− | 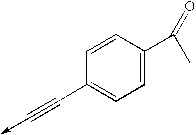 |
| 1390 1391 1392 | rac +− | 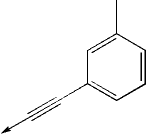 |
| 1393 1394 1395 | rac +− | 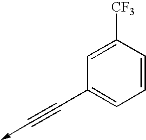 |
| 1396 1397 1398 | rac +− | 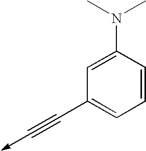 |
| 1399 1400 1401 | rac +− | 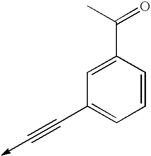 |
| 1402 1403 1404 | rac +− | 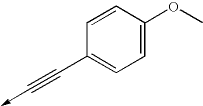 |
| 1405 1406 1407 | rac +− | 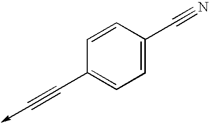 |
| 1408 1409 1410 | rac +− | 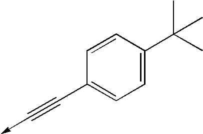 |
| 1411 1412 1413 | rac +− | 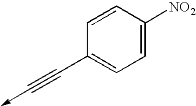 |
| 1414 1415 1416 | rac +− | 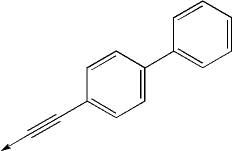 |
| 1417 1418 1419 | rac +− | 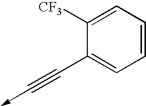 |
| 1420 1421 1422 | rac +− | 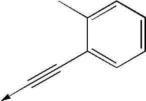 |
| 1423 1424 1425 | rac +− | 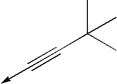 |
| 1426 1427 1428 | rac +− | 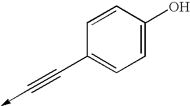 |

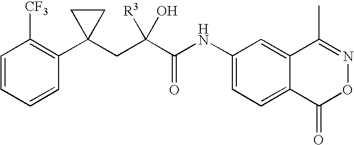
| | | |
|---|---|---|
| 1429 1430 1431 | rac −+ | 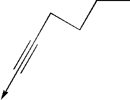 |
| 1432 1433 1434 | rac +− | 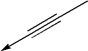 |
| 1435 1436 1437 | rac +− | 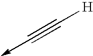 |
| 1438 1439 1440 | rac +− | 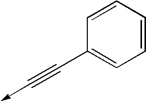 |
| 1441 1442 1443 | rac +− | 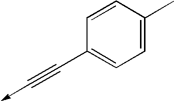 |
| 1444 1445 1446 | rac +− | 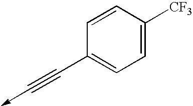 |
| 1447 1448 1449 | rac +− | 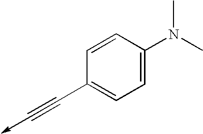 |
| 1450 1451 1452 | rac +− | 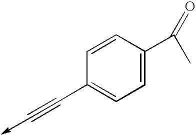 |
| 1453 1454 1455 | rac +− | 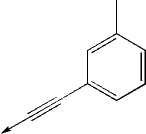 |
| 1456 1457 1458 | rac +− | 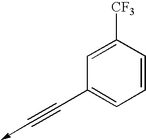 |
| 1459 1460 1461 | rac +− | 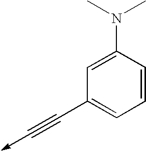 |
| 1462 1463 1464 | rac +− | 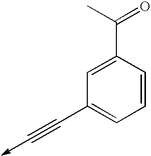 |
| 1465 1466 1467 | rac +− | 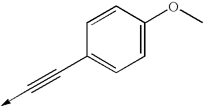 |
| 1468 1469 1470 | rac +− | 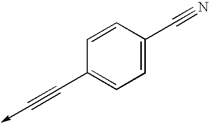 |
| 1471 1472 1473 | rac +− | 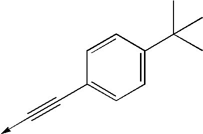 |
| 1474 1475 1476 | rac +− | 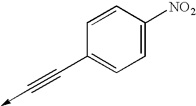 |
| 1477 1478 1479 | rac +− | 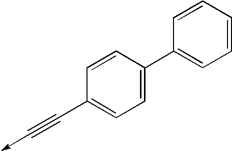 |
| 1480 1481 1482 | rac +− | 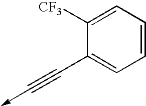 |
| 1483 1484 1485 | rac +− | 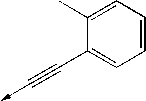 |
| 1486 1487 1488 | rac +− | 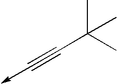 |
| 1489 1490 1491 | rac +− | 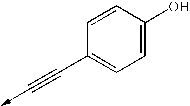 |

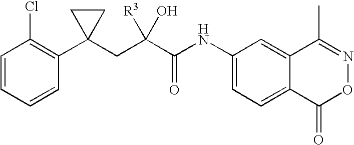
| | | |
|---|---|---|
| 1492 1493 1494 | rac −+ | 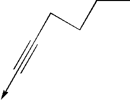 |
| 1495 1496 1497 | rac +− | 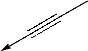 |
| 1498 1499 1500 | rac +− | 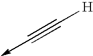 |
| 1501 1502 1503 | rac +− | 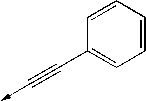 |
| 1504 1505 1506 | rac +− | 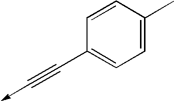 |
| 1507 1508 1509 | rac +− | 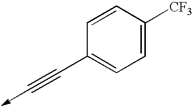 |
| 1510 1511 1512 | rac +− | 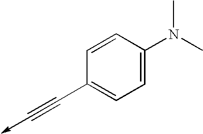 |
| 1513 1514 1515 | rac +− | 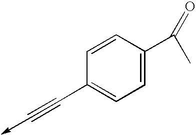 |
| 1516 1517 1518 | rac +− | 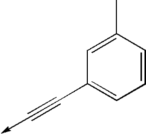 |
| 1519 1520 1521 | rac +− | 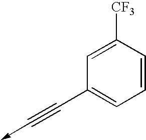 |
| 1522 1523 1524 | rac +− | 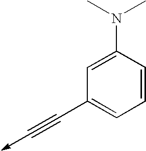 |
| 1525 1526 1527 | rac +− | 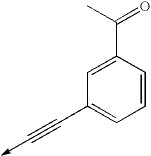 |
| 1528 1529 1530 | rac +− | 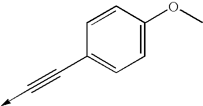 |
| 1531 1532 1533 | rac +− | 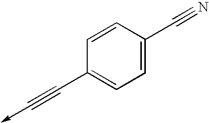 |
| 1534 1535 1536 | rac +− | 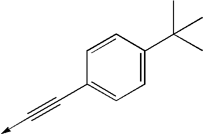 |
| 1537 1538 1539 | rac +− | 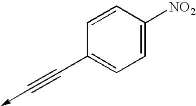 |
| 1540 1541 1542 | rac +− | 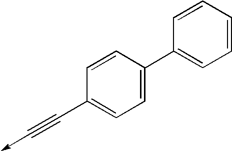 |
| 1543 1544 1545 | rac +− | 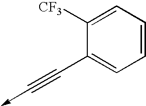 |
| 1546 1547 1548 | rac +− | 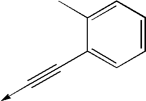 |
| 1549 1550 1551 | rac +− | 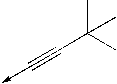 |
| 1552 1553 1554 | rac +− | 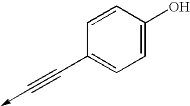 |

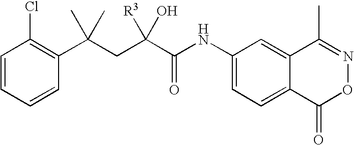
| | | |
|---|---|---|
| 1555 1556 1557 | rac −+ | 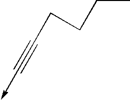 |
| 1558 1559 1560 | rac +− | 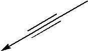 |
| 1561 1562 1563 | rac +− | 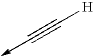 |
| 1564 1565 1566 | rac +− | 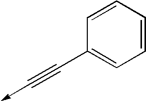 |
| 1567 1568 1569 | rac +− | 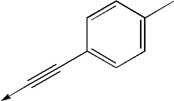 |
| 1570 1571 1572 | rac +− | 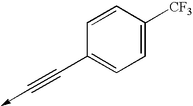 |
| 1573 1574 1575 | rac +− | 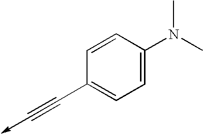 |
| 1576 1577 1578 | rac +− | 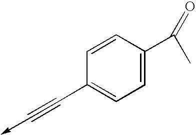 |
| 1579 1580 1581 | rac +− | 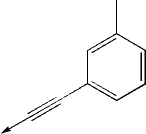 |
| 1582 1583 1584 | rac +− | 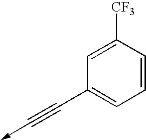 |
| 1585 1586 1587 | rac +− | 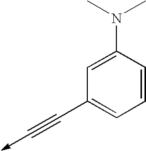 |
| 1588 1589 1590 | rac +− | 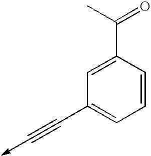 |
| 1591 1592 1593 | rac +− | 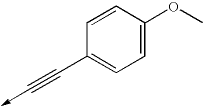 |
| 1594 1595 1596 | rac +− | 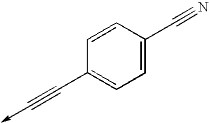 |
| 1597 1598 1599 | rac +− | 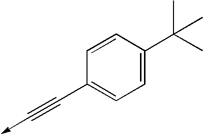 |
| 1600 1601 1602 | rac +− | 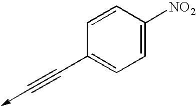 |
| 1603 1604 1605 | rac +− | 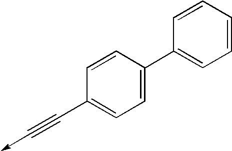 |
| 1606 1607 1608 | rac +− | 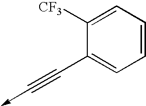 |
| 1609 1610 1611 | rac +− | 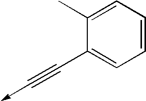 |
| 1612 1613 1614 | rac +− | 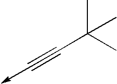 |
| 1615 1616 1617 | rac +− | 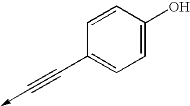 |

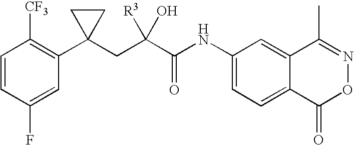
| | | |
|---|---|---|
| 1618 1619 1620 | rac −+ | 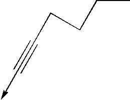 |
| 1621 1622 1623 | rac +− | 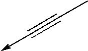 |
| 1624 1625 1626 | rac +− | 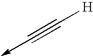 |
| 1627 1628 1629 | rac +− | 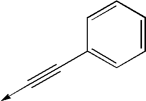 |
| 1630 1631 1632 | rac +− | 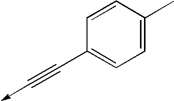 |
| 1633 1634 1635 | rac +− | 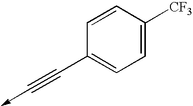 |
| 1636 1637 1638 | rac +− | 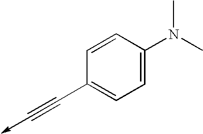 |
| 1639 1640 1641 | rac +− | 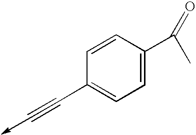 |
| 1642 1643 1644 | rac +− | 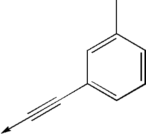 |
| 1645 1646 1647 | rac +− | 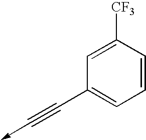 |
| 1648 1649 1650 | rac +− | 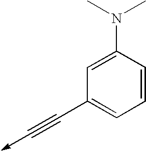 |
| 1651 1652 1653 | rac +− | 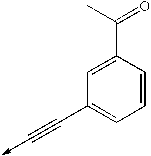 |
| 1654 1655 1656 | rac +− | 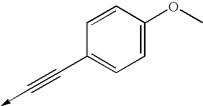 |
| 1657 1658 1659 | rac +− | 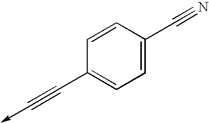 |
| 1660 1661 1662 | rac +− | 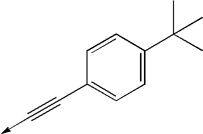 |
| 1663 1664 1665 | rac +− | 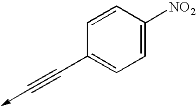 |
| 1666 1667 1668 | rac +− | 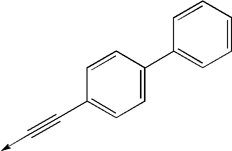 |
| 1669 1670 1671 | rac +− | 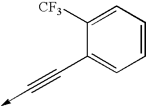 |
| 1672 1673 1674 | rac +− | 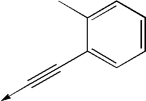 |
| 1675 1676 1677 | rac +− | 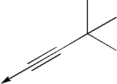 |
| 1678 1679 1680 | rac +− | 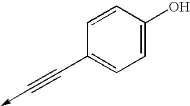 |

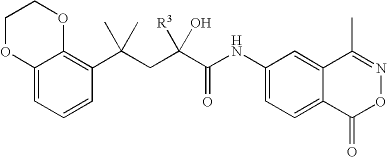
| | | |
|---|---|---|
| 1681 1682 1683 | rac −+ | 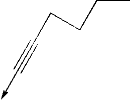 |
| 1684 1685 1686 | rac +− | 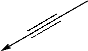 |
| 1687 1688 1689 | rac +− | 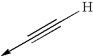 |
| 1690 1691 1692 | rac +− | 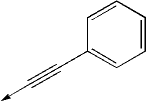 |
| 1693 1694 1695 | rac +− | 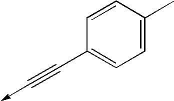 |
| 1696 1697 1698 | rac +− | 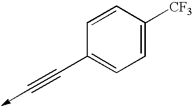 |
| 1699 1700 1701 | rac +− | 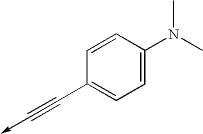 |
| 1702 1703 1704 | rac +− | 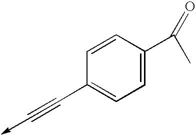 |
| 1705 1706 1707 | rac +− | 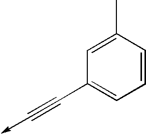 |
| 1708 1709 1710 | rac +− | 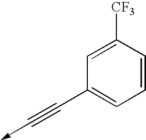 |
| 1711 1712 1713 | rac +− | 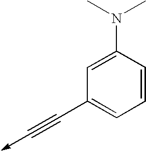 |
| 1714 1715 1716 | rac +− | 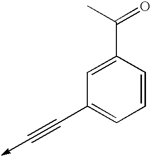 |
| 1717 1718 1719 | rac +− | 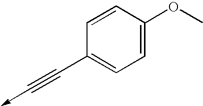 |
| 1720 1721 1722 | rac +− | 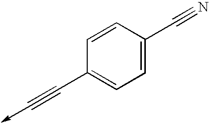 |
| 1723 1724 1725 | rac +− | 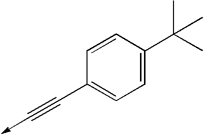 |
| 1726 1727 1728 | rac +− | 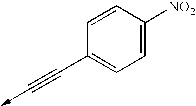 |
| 1729 1730 1731 | rac +− | 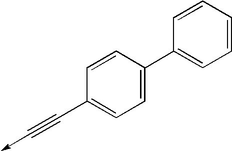 |
| 1732 1733 1734 | rac +− | 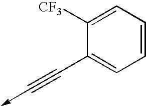 |
| 1735 1736 1737 | rac +− | 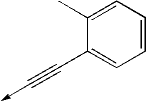 |
| 1738 1739 1740 | rac +− | 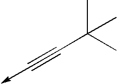 |
| 1741 1742 1743 | rac +− | 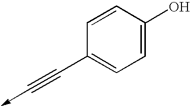 |
| 1744 1745 1746 | rac −+ | 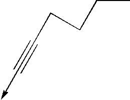 |
| 1747 1748 1749 | rac +− | 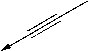 |
| 1750 1751 1752 | rac +− | 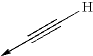 |
| 1753 1754 1755 | rac +− | 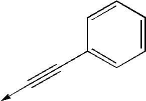 |
| 1756 1757 1758 | rac +− | 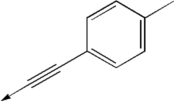 |
| 1759 1760 1761 | rac +− | 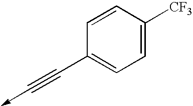 |
| 1762 1763 1764 | rac +− | 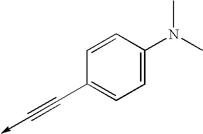 |
| 1765 1766 1767 | rac +− | 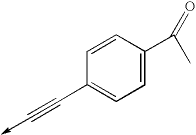 |
| 1768 1769 1770 | rac +− | 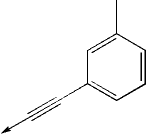 |
| 1771 1772 1773 | rac +− | 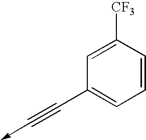 |
| 1774 1775 1776 | rac +− | 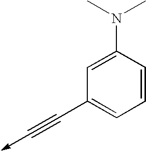 |
| 1777 1778 1779 | rac +− | 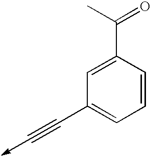 |
| 1780 1781 1782 | rac +− | 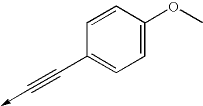 |
| 1783 1784 1785 | rac +− | 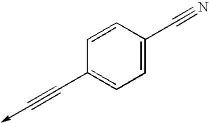 |
| 1786 1787 1788 | rac +− | 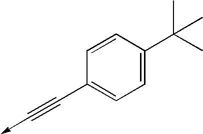 |
| 1789 1790 1791 | rac +− | 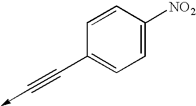 |
| 1792 1793 1794 | rac +− | 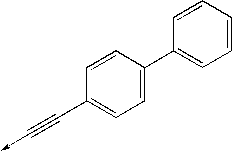 |
| 1795 1796 1797 | rac +− | 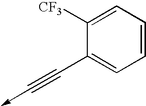 |
| 1798 1799 1800 | rac +− | 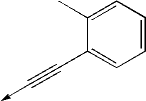 |
| 1801 1802 1803 | rac +− | 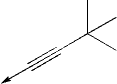 |
| 1804 1805 1806 | rac +− | 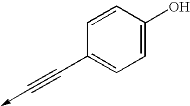 |
| 1807 1808 1809 | rac +− | 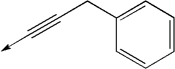 |
| 1810 1811 1812 | rac +− | 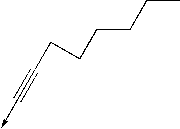 |

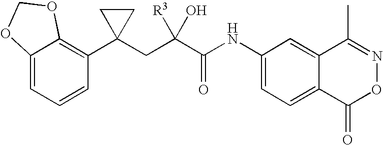
| | | |
|---|---|---|
| 1813 1814 1815 | rac −+ | 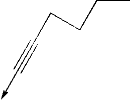 |
| 1816 1817 1818 | rac +− | 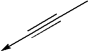 |
| 1819 1820 1821 | rac +− | 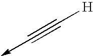 |
| 1822 1823 1824 | rac +− | 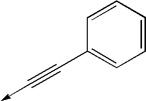 |
| 1825 1826 1827 | rac +− | 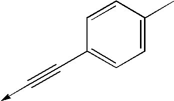 |
| 1828 1829 1830 | rac +− | 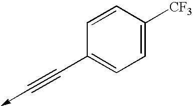 |
| 1831 1832 1833 | rac +− | 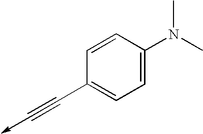 |
| 1834 1835 1836 | rac +− | 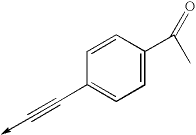 |
| 1837 1838 1839 | rac +− | 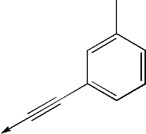 |
| 1840 1841 1842 | rac +− | 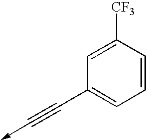 |
| 1843 1844 1845 | rac +− | 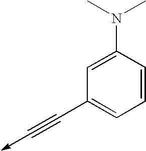 |
| 1846 1847 1848 | rac +− | 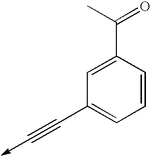 |
| 1849 1850 1851 | rac +− | 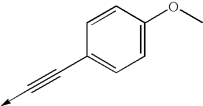 |
| 1852 1853 1854 | rac +− | 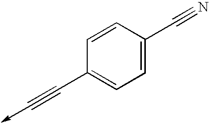 |
| 1855 1856 1857 | rac +− | 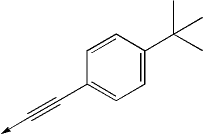 |
| 1858 1859 1860 | rac +− | 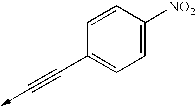 |
| 1861 1862 1863 | rac +− | 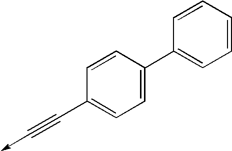 |
| 1864 1865 1866 | rac +− | 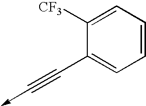 |
| 1867 1868 1869 | rac +− | 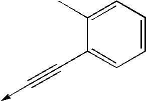 |
| 1870 1871 1872 | rac +− | 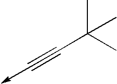 |
| 1873 1874 1875 | rac +− | 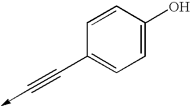 |

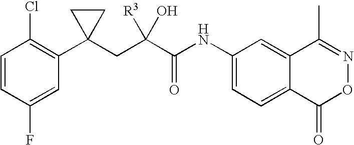
| | | |
|---|---|---|
| 1876 1877 1878 | rac −+ | 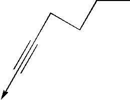 |
| 1879 1880 1881 | rac +− | 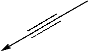 |
| 1882 1883 1884 | rac +− | 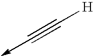 |
| 1885 1886 1887 | rac +− | 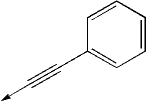 |
| 1888 1889 1890 | rac +− | 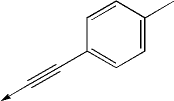 |
| 1891 1892 1893 | rac +− | 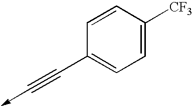 |
| 1894 1895 1896 | rac +− | 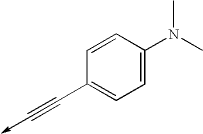 |
| 1897 1898 1899 | rac +− | 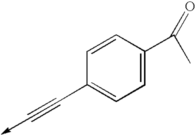 |
| 1900 1901 1902 | rac +− | 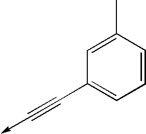 |
| 1903 1904 1905 | rac +− | 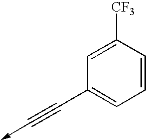 |
| 1906 1907 1908 | rac +− | 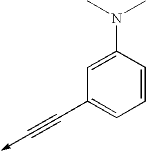 |
| 1909 1910 1911 | rac +− | 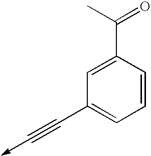 |
| 1912 1913 1914 | rac +− | 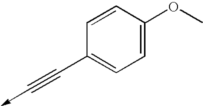 |
| 1915 1916 1917 | rac +− | 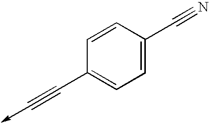 |
| 1918 1919 1920 | rac +− | 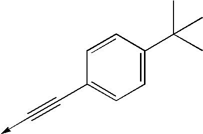 |
| 1921 1922 1923 | rac +− | 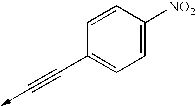 |
| 1924 1925 1926 | rac +− | 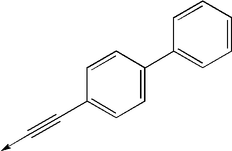 |
| 1927 1928 1929 | rac +− | 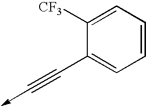 |
| 1930 1931 1932 | rac +− | 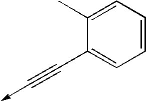 |
| 1933 1934 1935 | rac +− | 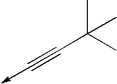 |
| 1936 1937 1938 | rac +− | 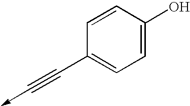 |

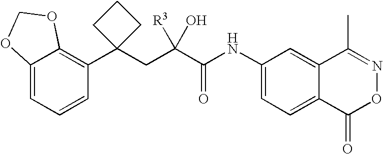
| | | |
|---|---|---|
| 1939 1940 1941 | rac −+ | 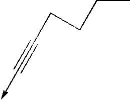 |
| 1942 1943 1944 | rac +− | 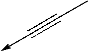 |
| 1945 1946 1947 | rac +− | 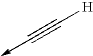 |
| 1948 1949 1950 | rac +− | 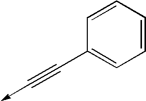 |
| 1951 1952 1953 | rac +− | 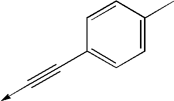 |
| 1954 1955 1956 | rac +− | 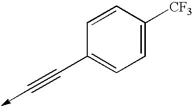 |
| 1957 1958 1959 | rac +− | 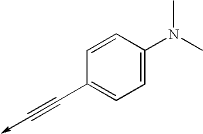 |
| 1960 1961 1962 | rac +− | 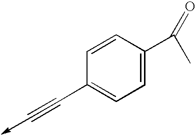 |
| 1963 1964 1965 | rac +− | 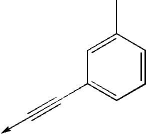 |
| 1966 1967 1968 | rac +− | 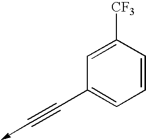 |
| 1969 1970 1971 | rac +− | 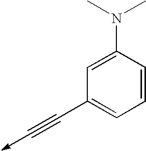 |
| 1972 1973 1974 | rac +− | 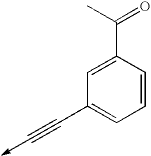 |
| 1975 1976 1977 | rac +− | 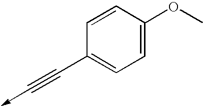 |
| 1978 1979 1980 | rac +− | 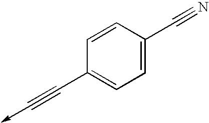 |
| 1981 1982 1983 | rac +− | 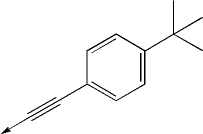 |
| 1984 1985 1986 | rac +− | 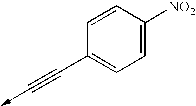 |
| 1987 1988 1989 | rac +− | 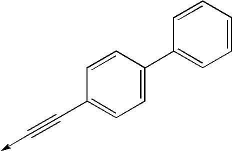 |
| 1990 1991 1992 | rac +− | 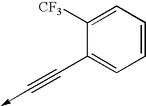 |
| 1993 1994 1995 | rac +− | 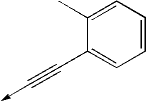 |
| 1996 1997 1998 | rac +− | 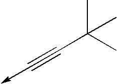 |
| 1999 2000 2001 | rac +− | 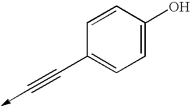 |

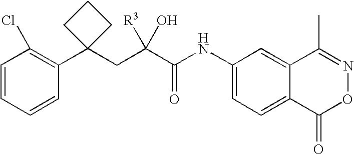
| | | |
|---|---|---|
| 2002 2003 2004 | rac −+ | 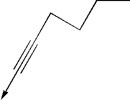 |
| 2005 2006 2007 | rac +− | 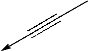 |
| 2008 2009 2010 | rac +− | 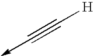 |
| 2011 2012 2013 | rac +− | 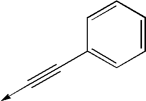 |
| 2014 2015 2016 | rac +− | 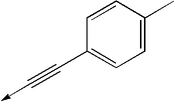 |
| 2017 2018 2019 | rac +− | 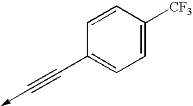 |
| 2020 2021 2022 | rac +− | 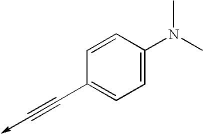 |
| 2023 2024 2025 | rac +− | 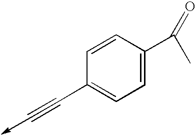 |
| 2026 2027 2028 | rac +− | 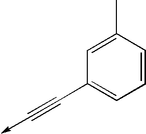 |
| 2029 2030 2031 | rac +− | 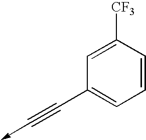 |
| 2032 2033 2034 | rac +− | 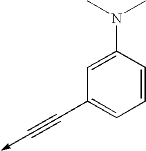 |
| 2035 2036 2037 | rac +− | 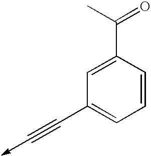 |
| 2038 2039 2040 | rac +− | 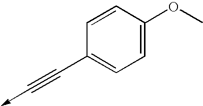 |
| 2041 2042 2043 | rac +− | 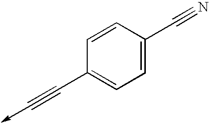 |
| 2044 2045 2046 | rac +− | 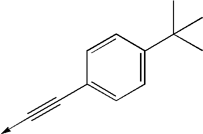 |
| 2047 2048 2049 | rac +− | 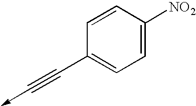 |
| 2050 2051 2052 | rac +− | 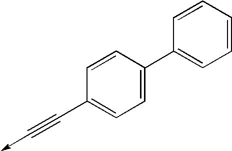 |
| 2053 2054 2055 | rac +− | 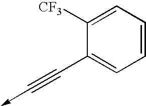 |
| 2056 2057 2058 | rac +− | 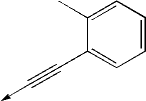 |
| 2059 2060 2061 | rac +− | 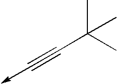 |
| 2062 2063 2064 | rac +− | 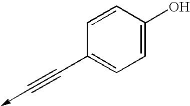 |

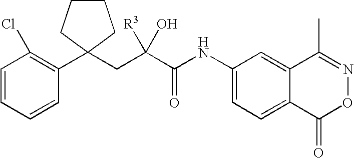
| | | |
|---|---|---|
| 2065 2066 2067 | rac −+ | 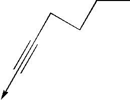 |
| 2068 2069 2070 | rac +− | 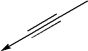 |
| 2071 2072 2073 | rac +− | 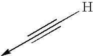 |
| 2074 2075 2076 | rac +− | 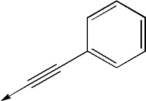 |
| 2077 2078 2079 | rac +− | 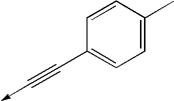 |
| 2080 2081 2082 | rac +− | 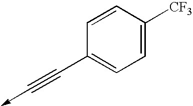 |
| 2083 2084 2085 | rac +− | 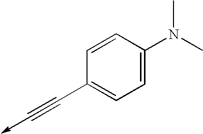 |
| 2086 2087 2088 | rac +− | 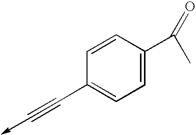 |
| 2089 2090 2091 | rac +− | 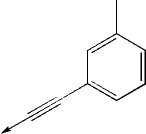 |
| 2092 2093 2094 | rac +− | 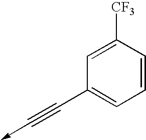 |
| 2095 2096 2097 | rac +− | 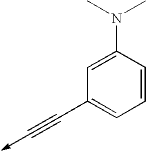 |
| 2098 2099 2100 | rac +− | 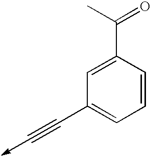 |
| 2101 2102 2103 | rac +− | 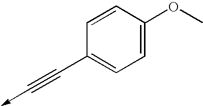 |
| 2104 2105 2106 | rac +− | 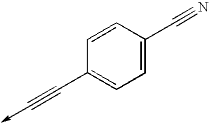 |
| 2107 2108 2109 | rac +− | 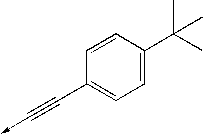 |
| 2110 2111 2112 | rac +− | 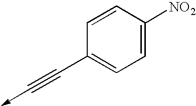 |
| 2113 2114 2115 | rac +− | 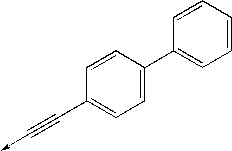 |
| 2116 2117 2118 | rac +− | 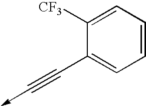 |
| 2119 2120 2121 | rac +− | 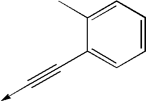 |
| 2122 2123 2124 | rac +− | 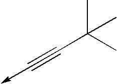 |
| 2125 2126 2127 | rac +− | 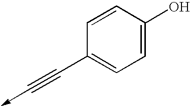 |

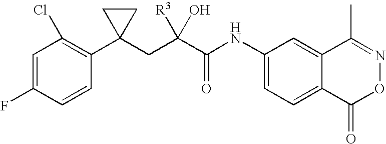
| | | |
|---|---|---|
| 2128 2129 2130 | rac −+ | 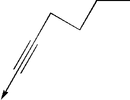 |
| 2131 2132 2133 | rac +− | 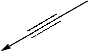 |
| 2134 2135 2136 | rac +− | 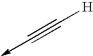 |
| 2137 2138 2139 | rac +− | 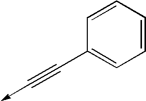 |
| 2140 2141 2142 | rac +− | 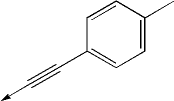 |
| 2143 2144 2145 | rac +− | 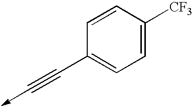 |
| 2146 2147 2148 | rac +− | 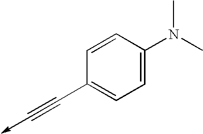 |
| 2149 2150 2151 | rac +− | 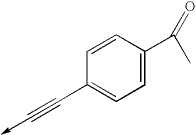 |
| 2152 2153 2154 | rac +− | 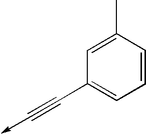 |
| 2155 2156 2157 | rac +− | 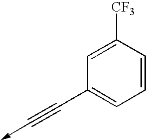 |
| 2158 2159 2160 | rac +− | 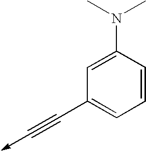 |
| 2161 2163 2163 | rac +− | 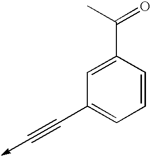 |
| 2164 2165 2166 | rac +− | 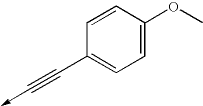 |
| 2167 2168 2169 | rac +− | 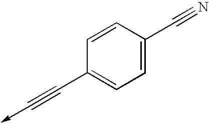 |
| 2170 2171 2172 | rac +− | 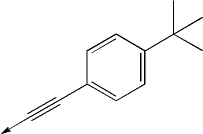 |
| 2173 2174 2175 | rac +− | 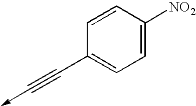 |
| 2176 2177 2178 | rac +− | 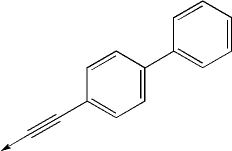 |
| 2179 2180 2181 | rac +− | 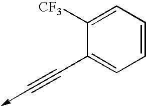 |
| 2182 2183 2184 | rac +− | 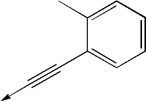 |
| 2185 2186 2187 | rac +− | 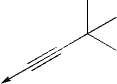 |
| 2188 2189 2190 | rac +− | 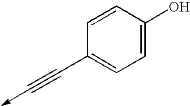 |

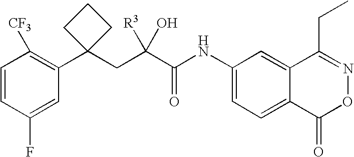
| | | |
|---|---|---|
| 2191 2192 2193 | rac −+ | 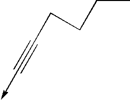 |
| 2194 2195 2196 | rac +− | 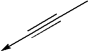 |
| 2197 2198 2199 | rac +− | 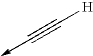 |
| 2200 2201 2202 | rac +− | 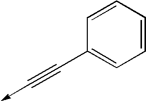 |
| 2203 2204 2205 | rac +− | 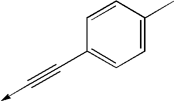 |
| 2206 2207 2208 | rac +− | 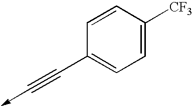 |
| 2209 2210 2211 | rac +− | 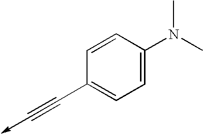 |
| 2212 2213 2214 | rac +− | 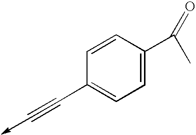 |
| 2215 2216 2217 | rac +− | 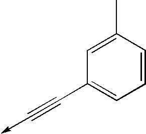 |
| 2218 2219 2220 | rac +− | 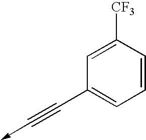 |
| 2221 2222 2223 | rac +− | 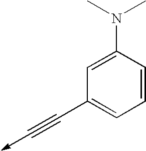 |
| 2224 2225 2226 | rac +− | 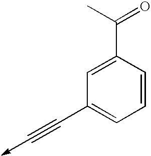 |
| 2227 2228 2229 | rac +− | 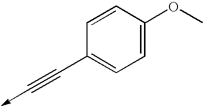 |
| 2230 2231 2232 | rac +− | 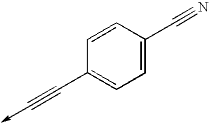 |
| 2233 2234 2235 | rac +− | 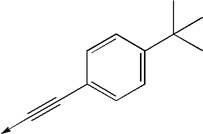 |
| 2236 2237 2238 | rac +− | 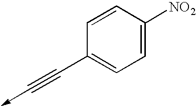 |
| 2239 2240 2241 | rac +− | 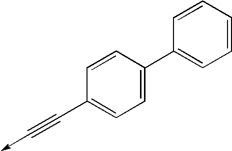 |
| 2242 2243 2244 | rac +− | 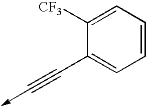 |
| 2245 2246 2247 | rac +− | 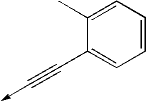 |
| 2248 2249 2250 | rac +− | 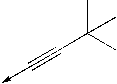 |
| 2251 2252 2253 | rac +− | 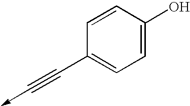 |

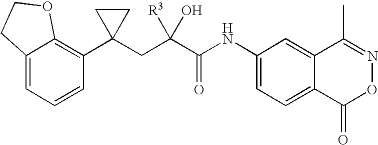
| | | |
|---|---|---|
| 2254 2255 2256 | rac −+ | 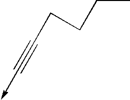 |
| 2257 2258 2259 | rac +− | 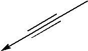 |
| 2260 2261 2262 | rac +− | 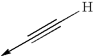 |
| 2263 2264 2265 | rac +− | 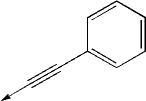 |
| 2266 2267 2268 | rac +− | 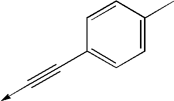 |
| 2269 2270 2271 | rac +− | 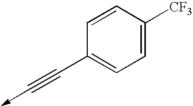 |
| 2272 2273 2274 | rac +− | 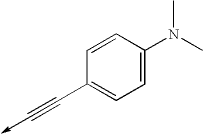 |
| 2273 2276 2277 | rac +− | 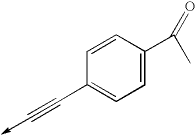 |
| 2278 2279 2280 | rac +− | 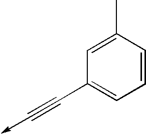 |
| 2281 2282 2283 | rac +− | 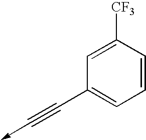 |
| 2284 2285 2286 | rac +− | 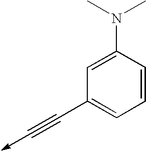 |
| 2287 2288 2289 | rac +− | 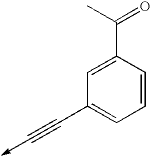 |
| 2290 2291 2292 | rac +− | 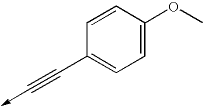 |
| 2293 2294 2295 | rac +− | 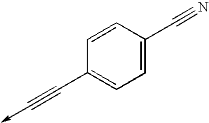 |
| 2296 2297 2298 | rac +− | 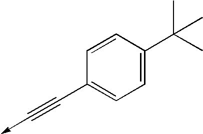 |
| 2299 2300 2301 | rac +− | 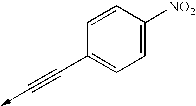 |
| 2302 2303 2304 | rac +− | 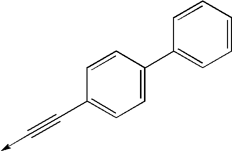 |
| 2305 2306 2307 | rac +− | 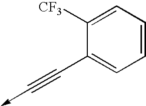 |
| 2308 2309 2310 | rac +− | 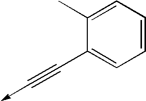 |
| 2311 2312 2313 | rac +− | 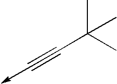 |
| 2314 2315 2316 | rac +− | 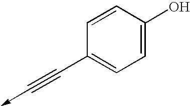 |

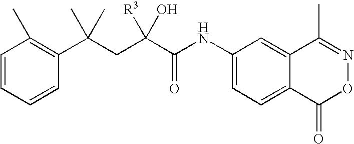
| | | |
|---|---|---|
| 2317 2318 2319 | rac −+ | 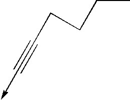 |
| 2320 2321 2322 | rac +− | 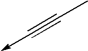 |
| 2323 2324 2325 | rac +− | 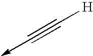 |
| 2326 2327 2328 | rac +− | 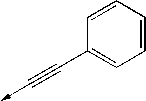 |
| 2329 2330 2331 | rac +− | 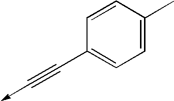 |
| 2332 2333 2334 | rac +− | 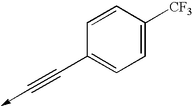 |
| 2335 2336 2337 | rac +− | 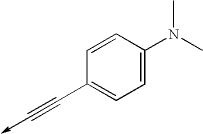 |
| 2338 2339 2340 | rac +− | 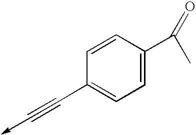 |
| 2341 2342 2343 | rac +− | 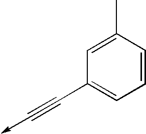 |
| 2344 2345 2346 | rac +− | 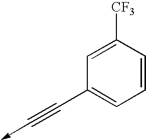 |
| 2347 2348 2349 | rac +− | 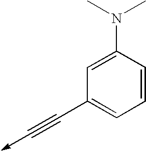 |
| 2350 2351 2652 | rac +− | 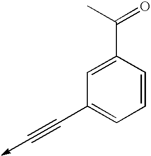 |
| 2353 2354 2355 | rac +− | 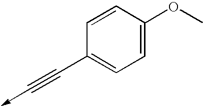 |
| 2356 2357 2358 | rac +− | 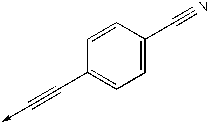 |
| 2359 2360 2361 | rac +− | 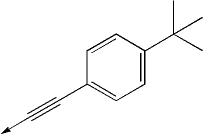 |
| 2362 2363 2364 | rac +− | 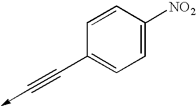 |
| 2365 2366 2367 | rac +− | 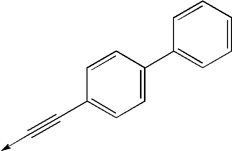 |
| 2368 2369 2370 | rac +− | 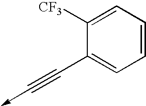 |
| 2371 2372 2373 | rac +− | 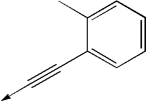 |
| 2374 2375 2376 | rac +− | 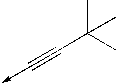 |
| 2377 2378 2379 | rac +− | 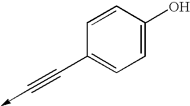 |

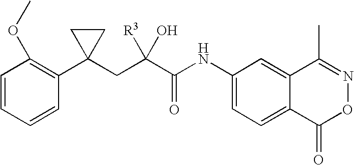
| | | |
|---|---|---|
| 2380 2381 2382 | rac −+ | 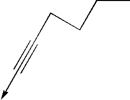 |
| 2383 2384 2385 | rac +− | 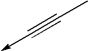 |
| 2386 2387 2388 | rac +− | 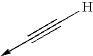 |
| 2389 2390 2391 | rac +− | 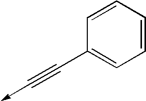 |
| 2392 2393 2394 | rac +− | 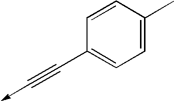 |
| 2395 2396 2397 | rac +− | 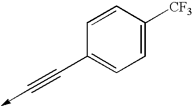 |
| 2398 2399 2400 | rac +− | 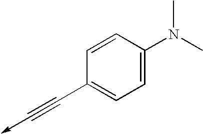 |
| 2401 2402 2403 | rac +− | 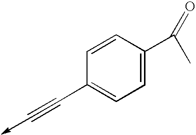 |
| 2404 2405 2406 | rac +− | 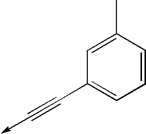 |
| 2407 2408 2409 | rac +− | 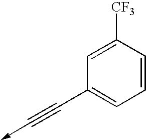 |
| 2410 2411 2412 | rac +− | 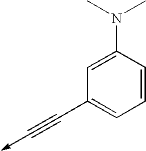 |
| 2413 2414 2415 | rac +− | 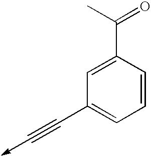 |
| 2416 2417 2418 | rac +− | 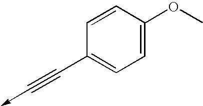 |
| 2419 2420 2421 | rac +− | 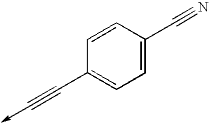 |
| 2422 2423 2424 | rac +− | 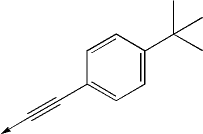 |
| 2425 2426 2427 | rac +− | 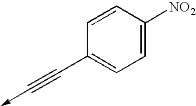 |
| 2428 2429 2430 | rac +− | 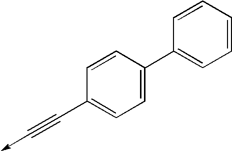 |
| 2431 2432 2433 | rac +− | 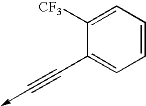 |
| 2434 2435 2436 | rac +− | 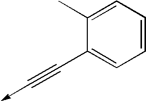 |
| 2437 2438 2439 | rac +− | 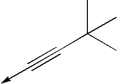 |
| 2440 2441 2442 | rac +− | 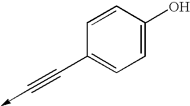 |
| 2443 2444 2445 | rac −+ | 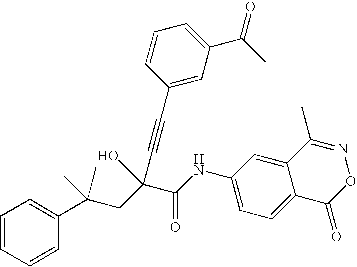 |
| 2446 2447 2448 | rac +− | 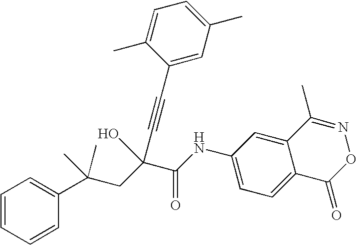 |
| 2449 2450 2451 | rac +− | 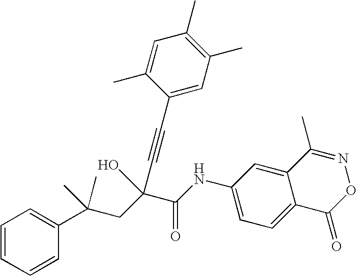 |
| 2452 2453 2454 | rac +− | 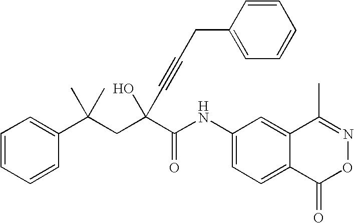 |
| 2455 2456 2457 | rac +− | 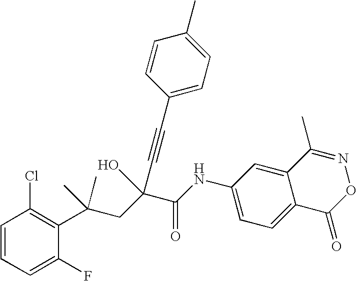 |
| 2458 2459 2460 | rac +− | 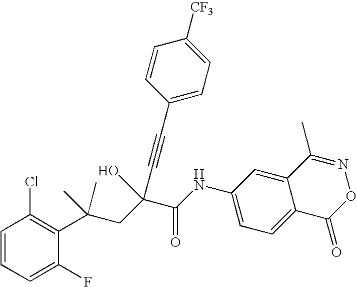 |
| 2461 2462 2463 | rac +− | 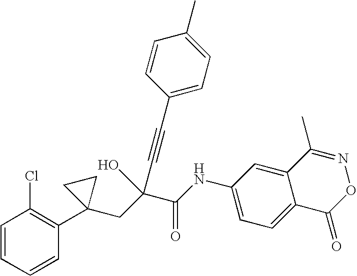 |
| 2464 2465 2466 | rac +− | 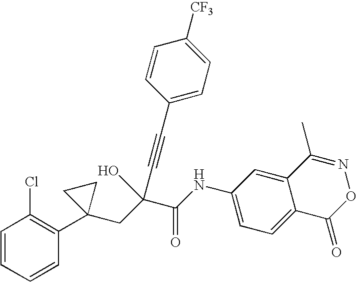 |
| 2467 2468 2469 | rac +− | 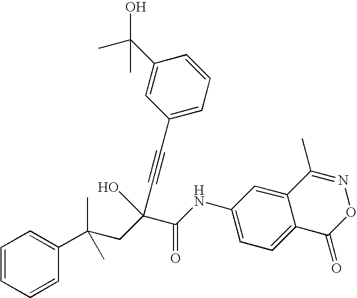 |
| 2470 2471 2472 | rac +− | 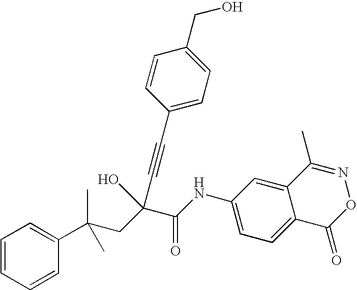 |
| 2473 2474 2475 | rac +− | 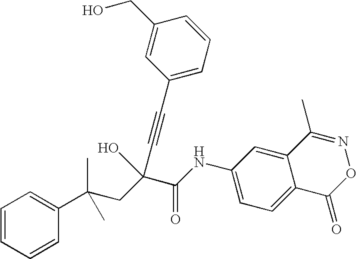 |
| 2476 2477 2478 | rac +− | 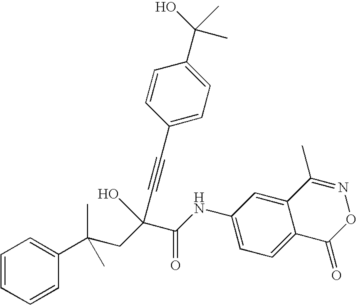 |

14. Pharmaceutical composition comprising at least one compound of the general formula I according to claim 1 and, where appropriate, at least one further active ingredient together with pharmaceutically suitable excipients and/or carriers.

15. Pharmaceutical composition according to claim 14, where the further active ingredient is a SERM (selective estrogen receptor modulator), an aromatase inhibitor, an antiestrogen or a prostaglandin.

16. Pharmaceutical composition according to claim 14, where the active ingredient may be tamoxifen, 5-(4-{5-[(RS)-(4,4,5,5,5-pentafluoropentyl)sulphinyl]pentyloxy}phenyl) -6-phenyl-8,9-dihydro-7H-benzocyclohepten-2-ol, ICI 182 780 (7alpha-[9-(4,4,5,5-pentafluoropentylsulphinyl)nonyl]estra-1,3,5(10)-triene-3,17beta-diol), 11beta-fluoro-7alpha-[5-(methyl {3-[(4,4,5,5,5-pentafluoropentyl)sulphanyl]propyl}amino)pentyl]estra-1,3,5(10)-triene -3,17beta-diol, 11beta-fluoro-7alpha-{5-[methyl(7,7,8,8,9,9,10,10,10-nonafluorodecyl)amino]pentyl}estra-1,3,5(10)-triene-3,17beta-diol, 11beta-fluoro-17alpha-methyl -7alpha-{5-[methyl(8,8,9,9,9-pentafluorononyl)amino]pentyl}estra-1,3,5(10)-triene -3,17beta-diol, clomifen, raloxifen, fadrozole, formestane, letrozole, anastrozole or atamestane.

17. Use of compounds according to claim 1 for producing a medicament.

18. Use of compounds according to claim 17 for producing a medicament for the therapy and prophylaxis of gynaecological disorders such as endometriosis, leiomyomas of the uterus, dysfunctional bleeding and dysmenorrhoea.

19. Use of compounds according to claim 17 for producing a medicament for the therapy and prophylaxis of hormone-dependent tumours.

20. Use of compounds according to claim 17 for producing a medicament for the therapy and prophylaxis of breast carcinomas.

21. Use of compounds according to claim 17 for producing a medicament for the therapy and prophylaxis of endometrial carcinoma.

22. Use of compounds according to claim 17 for producing a medicament for the therapy and prophylaxis of ovarian carcinomas.

23. Use of compounds according to claim 17 for producing a medicament for the therapy and prophylaxis of prostate carcinomas.

24. Use of compounds according to claim 17 for producing a medicament for female hormone replacement therapy.

25. Use of compounds according to claim 17 for female fertility control.

26. Process for the selective addition of lithium alkynyl and magnesium haloalkynyl compounds onto a keto amide.
